(19) United States
(12) Patent Application Publication
(10) Pub. No.: US 2025/0090727 A1
(43) Pub. Date: Mar. 20, 2025

(54) INTEGRATED BIOMATERIAL SCAFFOLD WITH 3D PRINTED LATTICE BACKBONE

(71) Applicant: Trustees of Dartmouth College, Hanover, NH (US)

(72) Inventors: Peter Bertone, Brookline, NH (US); Levi Olevsky, Lebanon, NH (US); Katherine Hixon, Hanover, NH (US)

(21) Appl. No.: 18/784,881

(22) Filed: Jul. 25, 2024

Related U.S. Application Data
(60) Provisional application No. 63/528,897, filed on Jul. 25, 2023.

(51) Int. Cl. A61L 27/56; A61F 2/28; A61L 27/26; B33Y 80/00; C08L 5/08; C08L 89/06

(52) U.S. Cl. CPC A61L 27/56; A61F 2/28; A61L 27/26; B33Y 80/00; C08L 5/08; C08L 89/06; A61F 2002/2835; A61F 2310/00215; A61F 2310/00221; A61F 2310/00293; A61L 2430/02

(57) ABSTRACT

A bone graft scaffold including the combination of a lattice structure having a triply periodic minimal surface (TPMS) shape and a cryogel solution disposed within the lattice structure. Also described is a method of making an integrated bone graft scaffold by freezing a cryogel solution, pouring the cryogel solution onto a scaffold, and freezing the cryogel solution poured onto the lattice structure to form an integrated bone graft scaffold. The cryogel solution may be a chitosan-gelatin cryogel and the lattice structure may be a 3D printed gyroid lattice structure.

### CROSS REFERENCE TO RELATED APPLICATIONS

This application claims priority to U.S. Provisional Patent Application No. 63/528,897, filed on Jul. 25, 2023, the disclosure of which is incorporated herein by reference.

### BACKGROUND OF THE DISCLOSURE

There is a high incidence of bone disorders caused by infection, trauma, or tumor-resection, highlighting a need for improved treatments to induce bone regeneration. Bone cements, synthetic bone fillers, synthetic bone grafts, and bone grafts may be used to treat bone disorders. Bone cements are typically used to fill in bone defects, but they often lack regenerative capabilities. Bone cements are often brittle and can fracture over time. Further, bone cements typically do not regenerate bone tissue and may be cytotoxic, promoting bone resorption. Even further, bone cement may cause an exothermic reaction when applied, burning surrounding healthy cells, and often takes a while to set in the human body, increasing the likelihood of infection. Synthetic bone fillers and synthetic bone grafts are often made from materials like calcium phosphate or hydroxyapatite, which can be effective in promoting bone growth. However, these methods are not tunable nor customizable which limits their application.

Bone grafting is a current treatment for defects less than 6 cm. Despite being a commonly used treatment, bone grafting has several limitations. Harvesting bone from a donor site can lead to associated complications such as pain, infection, and nerve damage. Additionally, the limited supply of donor tissue can be a challenge, particularly in cases where multiple grafts or repeat surgeries are required. Furthermore, bone grafts may fail to fully integrate with the surrounding tissue, leading to poor mechanical stability and requiring additional surgeries. These limitations have motivated the development of alternative treatments for bone regeneration.

Tissue engineering is an interdisciplinary field that involves the integration of biomaterial scaffolds, cells, and bioactive factors to promote targeted growth and regeneration of new tissue. Therefore, the implementation of a tissue-engineered scaffold framework that supports cell proliferation, migration, and attachment could present a promising substitute for bone grafting. While the clinical and economic advantages of tissue engineering are recognized, there are still areas that require attention to enhance translation from bench to bedside. In particular, the optimization of patient-specific biomaterials to mimic physical properties of bone represents a challenge. Specifically, bone tissue possesses unique mechanical properties, including stiffness, strength, and toughness, which enable it to support and protect the body.

Previous examples have explored various scaffold fabrication techniques to target bone formation. Cryogels scaffolds are produced by the freezing and subsequent thawing of a polymer solution, resulting in a sponge-like, macroporous structure that is ideal for cellular infiltration and angiogenesis. Further, natural materials including chitosan and gelatin can be incorporated to increase biocompatibility, biodegradability, and non-toxicity. Chitosan-gelatin cryogels are a popular choice for bone tissue engineering due to their biocompatibility and ideal physical properties such as pore size, swelling potential, and compressive moduli. Cryogels possess a high modulus of resilience, allowing them to be highly compressed without permanent deformation; however, they have a low modulus of elasticity, making these scaffolds mechanically weak and unable to bear high loads. This creates complications in using these scaffolds for large defects which deters the clinical use of cryogels.

Comparatively, 3D printing is another method for producing tissue-engineered scaffolds as it allows for the detailed printing of patient-specific geometries. This method provides a wide range of options for material selection, enabling the customization of scaffolds to achieve desired properties, and in the case of bone grafting, increased mechanical strength. Despite these advantages, the limited printing resolution of commonly available 3D printers remains a drawback in achieving the microstructure necessary for adequate tissue regeneration, where ideal pore diameters may range from 100-200 μm. The resolution of commonly used 3D printers ranges from 70-250 μm.

Therefore, there is a need for a bone graft that is biocompatible and provides a mechanically stable environment for bone tissue regeneration, while also promoting differentiation and proliferation of bone cells.

### SUMMARY OF THE DISCLOSURE

While both cryogels and 3D printing have been used in tissue engineering separately, the direct integration of these two methods has yet to be explored. By combining these two techniques, the resulting bone graft scaffold may take advantage of the benefits of both methods, including the high porosity and cellular infiltration of cryogels and the customization and mechanical strength of 3D printing.

The present disclosure provides a bone graft scaffold produced by the combination of two scaffold fabrication methods, cryogels and 3D printing, to create a composite bone graft scaffold with high porosity to ensure healthy cellular growth, while increasing mechanical durability to applied loads by the surrounding in vivo tissues. The process of cryogellation increases in difficulty and success with the additional of materials, due to the variance in material properties such as insulation, nucleation sites, etc. Embodiments of the present disclosure provide parameters for a bone graft scaffold that allows for the increased success rate of proper pore morphology of cryogellation. For example, in embodiments, the combination of the lattice structure dimensions, such as thickness, and the method of fabricating the bone graft scaffold as disclosed herein, such as the mixing, pouring, and freezing of the cryogel solution, as disclosed herein, allows for appropriate and consistent cryogellation of the composite materials.

The combination of cryogels with 3D printed gyroids provides a mechanically stable, macroporous structure to support the creation of patient-specific bone graft scaffolds for complex bone defects. To appropriately combine these two scaffold fabrication methods, the 3D printed framework may interface with the cryogel, while increasing mechanical strength of the combination bone graft scaffold and supporting cellular adhesion and proliferation.

The composite bone graft scaffold of the present disclosure retains similar porosity and relative swelling ratio to control cryogels while increasing the mechanical strength, as compared to other scaffold structures.

The present disclosure provides an integrated, mechanically strong bone graft scaffold that promotes cellular adhesion.

The present disclosure provides a bone graft scaffold having a gyroid lattice shape. The gyroid lattice shape has a highly interconnected porous structure, providing a large surface area for cell attachment and proliferation, efficient transport of nutrients and waste, and controlled mechanical properties. The gyroid lattice may be composed of repeating triply periodic minimal surfaces, which may be generated through mathematical algorithms and fabricated using advanced manufacturing techniques, such as 3D printing. The gyroid lattice may be tuned to match the mechanical properties of the surrounding tissue and promote cell differentiation by varying the strut thickness, pore size, and overall dimensions. The gyroid lattice shape may provide a favorable microenvironment for cell attachment, proliferation, and differentiation, as well as angiogenesis, due to its high surface area-to-volume ratio. The gyroid lattice shape structure may promote bone regeneration and improve implant integration.

The present disclosure provides a structure having a lattice shape. The lattice may be a structure with highly interconnected pores, which provide a large surface area for cell attachment and proliferation. The lattice structure may be tuned to match the mechanical properties of the surrounding tissue, promoting cell differentiation and implant integration. This shape may also provide anisotropic mechanical properties, which can be used to match the directional alignment of the surrounding native tissue.

The present disclosure provides a method for creating patient-specific bone graft scaffolds for complex bone defects, by combining cryogels with 3D printed gyroids. The present disclosure may provide a customizable bone graft scaffold that can be tailored to the specific needs of individual patients. This may be useful for complex bone defects where multiple grafts or repeat surgeries are required or where the limited supply of donor tissue may be a challenge.

The present disclosure provides a bone graft scaffold that may include a lattice structure having a triply periodic minimal surface (TPMS) shape and a cryogel solution disposed within the lattice structure. In embodiments of the present disclosure, the lattice structure may be 3D printed.

In embodiments of the present disclosure, the lattice structure having the TPMS shape may be shaped as a gyroid lattice, a diamond, an I-WP, a neovius, a primitive, a Fischer-Koch S, an F-RD, or a PMY.

In embodiments of the present disclosure, the lattice structure may have a solid volume from 0.5 to 150 cm³. The range of the lattice structure and/or the bone graft scaffold may be approximately the size of common bone defects. This range can be optimized depending on the bone defect the bone graft scaffold is treating.

In embodiments of the present disclosure, the lattice structure may have a relative density from 10% to 50%. Embodiments of the range allow for a balance between the lattice structure and the cryogel solution volume.

In embodiments of the present disclosure, the lattice structure may have a pore diameter from 0.5 to 5 mm.

In embodiments of the present disclosure, the lattice structure may be fabricated of ceramic materials or plastic materials. For example, the lattice structure may be fabricated from minerals, bone char, porcelain, and/or calcium metasilicate.

In embodiments of the present disclosure, the ceramic materials may be inorganic composites comprising calcium, phosphorus, silicon dioxides, and/or calcium carbonates.

In embodiments of the present disclosure, the minerals may be one or more of hydroxyapatite (Ca₁₀(PO₄)6(OH)), Wollastonite (CaSiO₃), and/or β-TCP (Ca₃(PO₄)).

In embodiments of the present disclosure, the cryogel solution may be made of at least one of chitosan, gelatin, silk, collagen, polyacrylamide, alginate, cellulose, laminin, fibrinogen, MXenes, polyethylene glycol (PEG), polyethylene oxide (PEO), polyvinyl alcohol (PVA), or N-Vinylpyrrolidone (NVP).

In embodiments of the present disclosure, the cryogel solution may be a chitosan-gelatin cryogel solution.

In embodiments of the present disclosure, the cryogel solution may have a porosity from 50% to 80%.

In embodiments of the present disclosure, the bone graft scaffold may have a porosity from 25% to 70%. The porosity is a result of other parameters such as the volume of the cryogel solution and the lattice structure.

In embodiments of the present disclosure, the bone graft scaffold may have a relative swelling ratio from 1500% to 2500%.

In embodiments of the present disclosure, the bone graft scaffold may have a mechanical strength from 0.5 MPa to 100 MPa. In embodiments, a lower mechanical strength may not be stiff enough for grafting. Further, a higher mechanical strength may cause stress shielding.

Further, the present disclosure provides a method of making a bone graft scaffold that may include freezing a cryogel solution, pouring the cryogel solution onto a lattice structure having a triply periodic minimal surface (TPMS) shape, and freezing the cryogel solution poured onto the lattice structure to form an integrated bone graft scaffold. In embodiments of the present disclosure, the lattice structure may be 3D printed.

In embodiments of the present disclosure, the method of making the bone graft scaffold may further comprise sintering the bone graft scaffold.

In embodiments of the present disclosure, the cryogel solution may be made of at least one of chitosan, gelatin, silk, collagen, polyacrylamide, alginate, cellulose, laminin, fibrinogen, MXenes, polyethylene glycol (PEG), polyethylene oxide (PEO), polyvinyl alcohol (PVA), or N-Vinylpyrrolidone (NVP).

In embodiments of the present disclosure, the cryogel solution may be a chitosan-gelatin cryogel solution.

In embodiments of the present disclosure, the cryogel solution may have a porosity from 50% to 80%.

In embodiments of the present disclosure, the lattice structure having the TPMS shape may be shaped as a gyroid lattice, a diamond, an I-WP, a neovius, a primitive, a Fischer-Koch S, an F-RD, or a PMY.

In embodiments of the present disclosure, the lattice structure may have a solid volume from 0.5 to 150 cm³. The range of the lattice structure and/or the bone graft scaffold may be approximately the size of common bone defects. This range can be optimized depending on the bone defect the bone graft scaffold is treating.

In embodiments of the present disclosure, the lattice structure may have a relative density from 10% to 50%. Embodiments of the range allow for a balance between the lattice structure and the cryogel solution volume.

In embodiments of the present disclosure, the lattice structure may have a pore diameter from 0.5 to 5 mm.

In embodiments of the present disclosure, the lattice structure may be fabricated of ceramic materials or plastic materials. For example, the lattice structure may be fabricated from minerals, bone char, porcelain, and/or calcium metasilicate.

In embodiments of the present disclosure, the ceramic materials may be inorganic composites comprising calcium, phosphorus, silicon dioxides, and/or calcium carbonates.

In embodiments of the present disclosure, the minerals may be one or more of hydroxyapatite (Ca₁₀(PO₄)6(OH)), Wollastonite (CaSiO₃), and/or β-TCP (Ca₃(PO₄)).

In embodiments of the present disclosure, the integrated bone graft scaffold may have a porosity from 25% to 70%. The porosity is a result of other parameters such as the volume of the cryogel solution and the lattice structure.

In embodiments of the present disclosure, the integrated bone graft scaffold may have a relative swelling ratio from 1500% to 2500%.

In embodiments of the present disclosure, the bone graft scaffold may have a mechanical strength from 0.5 MPa to 100 MPa. In embodiments, a lower mechanical strength may not be stiff enough for grafting. Further, a higher mechanical strength may cause stress shielding.

In embodiments of the present disclosure, the freezing the cryogel may include freezing the cryogel at a temperature from −10° C. to −80° C. for a duration from 30 minutes to 24 hours.

In embodiments of the present disclosure, the freezing the cryogel solution poured onto the lattice structure may include a freeze-drying cycle using a lyophilizer for a duration from 6 hours to 48 hours.

### BRIEF DESCRIPTION OF THE FIGURES

For a fuller understanding of the nature and objects of the disclosure, reference should be made to the following detailed description taken in conjunction with the accompanying figures.

FIG. 1 displays an embodiment of the combination of a 3D printed mineral lattice structure and a cryogel, to form an embodiment of the bone graft scaffold as disclosed herein.

FIG. 2A displays an embodiment of the cryogellation and fabrication method of an embodiment of the bone graft scaffold as disclosed herein.

FIG. 2B displays an embodiment of the cryogellation and fabrication method of an embodiment of the bone graft scaffold as disclosed herein, in a syringe.

FIG. 3 displays an embodiment of composite cryogel formulation, fabrication, and cryogellation process of the methods described herein.

FIG. 4 displays a biological application of an embodiment of the bone graft scaffold.

FIG. 5A displays an embodiment of a gyroid lattice structure.

FIG. 5B displays embodiments of a gyroid lattice structure.

FIG. 6A displays an embodiment of a 3D printed lattice structure.

FIG. 6B displays an embodiment of a lyophilized control cryogel.

FIG. 6C displays an embodiment of the bone graft scaffold as disclosed herein.

FIG. 7 displays SEM images of embodiments of the bone graft scaffolds and a control cryogel.

FIG. 8 displays results of a pore analysis of embodiments of the bone graft scaffolds with 2.0 mm shown on the left, 2.5 mm shown in the center, and 3.0 mm shown on the right for 10%, 20%, and 30% gyroid solidity.

FIG. 9A displays a Python generated pore analysis program output of an embodiment of a cryogel.

FIG. 9B displays a pore analysis of embodiments of the bone graft scaffold, where the dotted line is control value. There is a statistical difference between the pore diameters of the 30% 2.0 mm and 30% 3.0 mm bone graft scaffolds (P<0.05).

FIGS. 10A-10C display results of embodiments of the bone graft scaffold swelling with 3DP structure weights subtracted: 10% solidity bone graft scaffold (FIG. 10A), 20% solidity bone graft scaffold (FIG. 10B), and 30% solidity bone graft scaffold (FIG. 10C). 30% solidity bone graft scaffolds are significant against control values at every time point. 10% 2.0 mm bone graft scaffolds are significant from 10% 3.0 mm bone graft scaffolds at every time point, except at 20 minutes (P<0.05).

FIGS. 11A-11C display stress-strain results from a compression test of all bone graft scaffolds tested using an Instron 5544: 10% solidity bone graft scaffold (FIG. 11A), 20% solidity bone graft scaffold (FIG. 11B), and 30% solidity bone graft scaffold (FIG. 11C). The bone graft scaffolds were subjected to 80% compressive strain.

FIG. 12A displays an average compressive modulus of embodiments of the bone graft scaffolds calculated from Instron 68SC-2 with 2.0 mm shown on the left, 2.5 mm shown in the center, and 3.0 mm shown on the right for 10%, 20%, and 30% gyroid solidity.

FIG. 12B displays results of the average Young's modulus calculated from Instron 5544 compression test. Each solidity is significantly different from every other solidity (P<0.05). The control young's modulus is 0.571 MPa.

FIG. 13 displays cell infiltration-stained images using DAPI staining and light microscopy.

FIGS. 14A-14D display an embodiment of the hydroxyapatite lattice structure and accompanying SEM images thereof.

FIGS. 14E-14H display an embodiment of the bone graft scaffold and accompanying SEM images thereof.

FIGS. 15A-15C displays confocal images of embodiments of a mineral lattice structure and a cryogel.

FIGS. 16A-16B display SEM images of embodiments of the present disclosure with EDS software mapping and common elements found in embodiments of the present disclosure.

FIG. 17A displays a graphical representation of a stress-strain curve of an embodiment of the bone graft scaffold as disclosed herein.

FIG. 17B displays graphical representation of compressive moduli values of an embodiment of the bone graft scaffold as disclosed herein.

FIG. 17C displays graphical representation of the swelling capacity of an embodiment of the bone graft scaffold as disclosed herein.

FIGS. 18A-18F display images of the embodiments of the bone graft scaffold and the control scaffolds using a spinning disk confocal microscope.

FIGS. 19A-19H display embodiments of a hydroxyapatite lattice structure and accompanying SEM images thereof, and the composite scaffold and accompanying SEM images thereof.

FIGS. 20A-20B display FTIR curves of embodiments of printed lattice structures before sintering (FIG. 20A) and after sintering (FIG. 20B).

FIG. 21 displays a TG analysis of embodiments of pre-sintered printed lattice structures.

FIG. 22 displays images of embodiments of 3D printed lattice structures from different resins.

FIG. 23 displays SEM images of embodiments of 3D printed gyroid lattice structures from different resins, before and after sintering, at 15×.

FIG. 24 displays SEM images of embodiments of 3D printed gyroid lattice structures from different resins at 1000×.

FIG. 25 displays SEM images of embodiment of 3D printed gyroid lattice structures of different compositions, before and after sintering, at 2000×.

FIG. 26 displays macroscopic images of embodiments of β-TCP gyroid lattice structures before sintering (right) and after sintering (left).

FIG. 27 displays an ultimate compression of embodiments of gyroid lattice structures to compare changes in elastic moduli based on resin composition, before and after sintering.

FIG. 28A displays a comparison of elastic moduli between embodiments of lattice structures of different resin compositions before sintering.

FIG. 28B displays a comparison of elastic moduli between embodiments of lattice structures of different resin compositions after sintering.

FIG. 29 displays confocal microscopy images of embodiments of lattice structures from different resin compositions taken after a 4-day culture and a 7-day culture.

FIG. 30A displays SEM images of embodiments of the composite bone graft scaffold and the control, and crystal polymorph creation following soaking in Ca²⁺ and PO₄³⁻ solutions.

FIG. 30B displays absorbance values of ARS bound to calcium salts.

FIG. 31A displays compressive stress-strain curves of embodiments of the composite bone graft scaffold.

FIG. 31B displays Young's modulus values following compression tests for embodiments of the composite bone graft scaffold and control scaffolds.

FIG. 32A displays SEM images of the pores of an embodiment of the composite bone graft scaffold.

FIG. 32B displays pore diameter values of embodiments of the composite bone graft scaffold and control.

FIG. 32C displays comparison results of the swelling kinetics of embodiments of the composite bone graft scaffold and control.

FIG. 33 displays fluorescence microscopy of sectioned samples and surface level SEM images of embodiment of the bone graft scaffolds. Bone graft scaffolds were seeded with MG-63 osteoblast-like cells and incubated for 3-, 6-, 7-, 14- and 21-day timepoints.

FIG. 34A displays SEM images of MG-63 cells adhered to an embodiment of a cryogel.

FIG. 34B displays SEM images of MG-63 cells adhered to an embodiment of a 3D printed HA lattice structure.

FIG. 35 displays confocal fluorescence microscopy of a control, an embodiment of the composite bone graft scaffold, and a cryogel scaffold from a separate cell study.

FIG. 36 displays SEM images of embodiments of cryogels with additives, at 200×.

FIG. 37A displays a python generated pore analysis program output of an embodiment of a cryogel.

FIG. 37B displays results of a pore area distribution test of embodiments of cryogels with additives. The solid line is the mean value (p<0.05).

FIG. 38A displays compressive modulus of embodiments of cryogels with additives calculated from stress-strain curve generated by an Instron 68SC-2 system (p<0.05).

FIG. 38B displays the absorbance of embodiments of cryogels with additives calculated using ARS staining and SpectraMax Microplate Reader.

FIG. 39 displays cell infiltration-stained images using DAPI staining and light microscopy. Individual fluorescent dots indicate the nuclei of the MG-63 cells. The structures indicate an embodiment of the bone graft scaffold.

FIG. 40 displays SEM images of un-sintered and un-mineralized lattice structures.

FIGS. 41A-41B display EDS results of sintered lattice structures.

FIG. 42 displays results of a mineralization assay showing that sintered lattice structures have higher levels of mineralization (p<0.05).

FIG. 43A displays results of a lattice compression testing showing that unmineralized and unsintered lattice structures are the stiffest and strongest (p<0.05).

FIG. 43B displays a compressive stress-strain curve for four conditions; unsintered mineralized lattice structures, sintered mineralized lattice structures, sintered unmineralized lattice structures, and unsintered unmineralized lattice structures.

FIGS. 44A-44B display an FTIR analysis for sintered conditions with respective peaks (FIG. 44A), and unsintered conditions with respective peaks (FIG. 44B).

### DETAILED DESCRIPTION OF THE DISCLOSURE

Although claimed subject matter will be described in terms of certain embodiments, other embodiments, including embodiments that do not provide all of the benefits and features set forth herein, are also within the scope of this disclosure. Various structural, logical, process step, and electronic changes may be made without departing from the scope of the disclosure.

Ranges of values are disclosed herein. The ranges set out a lower limit value and an upper limit value. Unless otherwise stated, the ranges include all values to the magnitude of the smallest value (either lower limit value or upper limit value) and ranges between the values of the stated range.

The steps of the method described in the various embodiments and examples disclosed herein are sufficient to carry out the methods of the present disclosure. Thus, in an embodiment, the method consists essentially of a combination of the steps of the methods disclosed herein. In another embodiment, the method consists of such steps.

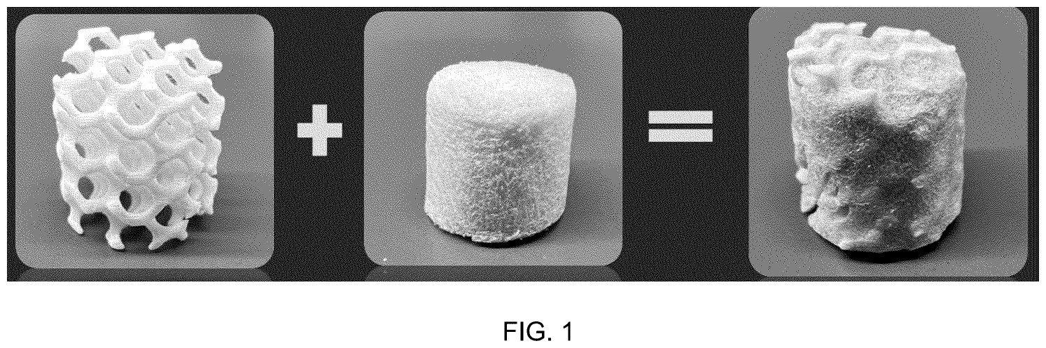

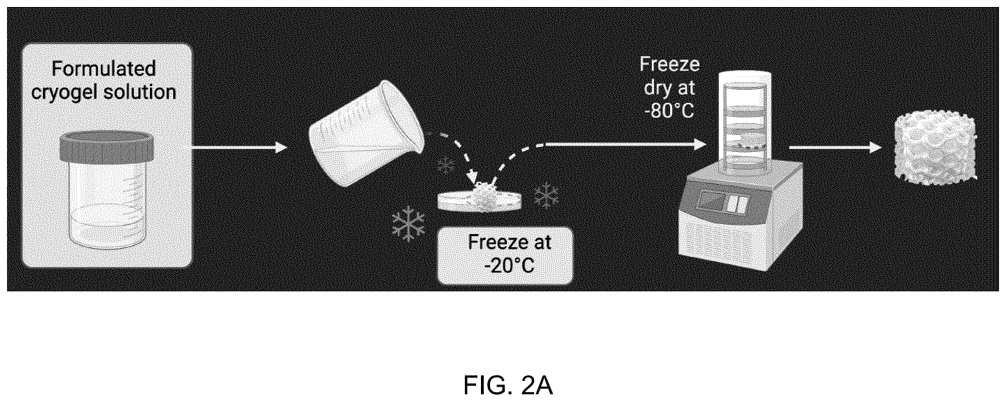

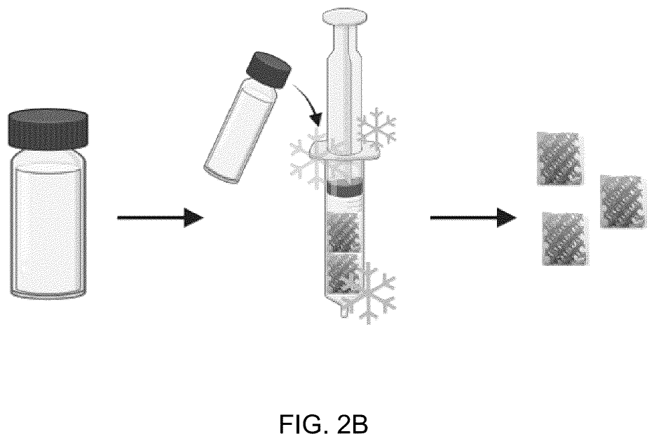

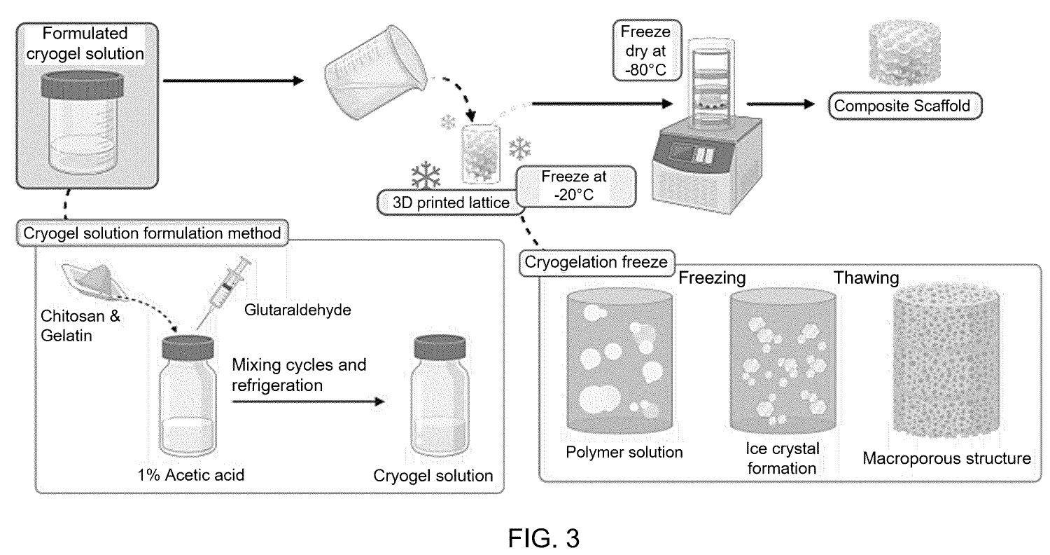

The present disclosure provides a synthetic bone graft scaffold to be used in orthopedic reconstruction. As shown in FIG. 1, the bone graft is a composite biomaterial structure composed of two distinct parts that are integrated during a freeze to thaw cycle, as shown in FIGS. 2A, 2B, and 3. As shown in FIG. 1, the first element is a 3D-printed gyroid lattice structure, which is shown on the left. This gyroid lattice structure can be an interconnected matrix with a high porosity to structural integrity ratio. This lattice matrix may be printed out of plastic materials, as well as ceramic resins such as porcelain and hydroxyapatite (Ca₁₀(PO₄)6(OH)) (calcium phosphate). The second element is a solution made of two polymers cross linked together, for example chitosan cross-linked to gelatin, which is shown in the middle of FIG. 1. When frozen and dried out, this solution forms a cryogel, a sponge-like material with interconnected pores, from 50 μm to 200 μm in diameter (e.g., approximately 120 μm in diameter). These parts can be combined into a bone graft scaffold, as shown on the right of FIG. 1.

As shown in FIG. 2A, in an embodiment of the present disclosure, the cryogel solution may be poured onto a lattice structure and undergo a freeze process at from −10° C. to −80° C. (e.g., −20° C.) for a period from 30 minutes to 24 hours (e.g., 18 hours), followed by a subsequent freeze-dry cycle using a lyophilizer for a period from 6 hours to 48 hours (e.g., 12 hours). Following the freezing, the solution is thawed. Through this method, the solution and lattice structure effectively integrate together to form the bone graft scaffold as disclosed herein. The bone graft scaffold may be referred to as a composite scaffold, a composite bone graft scaffold, a combined scaffold, or a combined bone graft scaffold.

FIG. 2B displays an embodiment of the freezing process used to fabricate embodiments of the present disclosure. In an embodiment, the cryogel solution may be made of 1% acetic acid, 1:4 chitosan—gelatin, and 1% glutaraldehyde. The solution may be poured onto a lattice structure within a syringe. The syringe may undergo a freeze process at from −10° C. to −80° C. (e.g., −20° C.) for a period from 30 minutes to 24 hours (e.g., 18 hours), followed by a subsequent freeze-dry cycle using a lyophilizer for a period from 6 hours to 48 hours (e.g., 12 hours). Embodiments may continue to freeze-dry for storage and may be rehydrated for later use.

FIG. 3 displays an embodiment of the freeze to thaw cycle disclosed herein. In an embodiment, the formulated cryogel solution may be formed by mixing chitosan and gelatin in 1% acetic acid. Further, glutaraldehyde may be added to the cryogel solution. These materials may go through mixing cycles and be refrigerated to form the cryogel solution. Further, FIG. 3 displays an embodiment of the cryogellation freeze as disclosed herein. In an embodiment, the polymer and crosslinker solutions undergo a freezing process. The freezing process may form ice crystals. During the thawing process, an interconnected macroporous structure is formed. This process allows for highly microporous and biocompatible bone graft scaffolds.

Traditional bone grafts, such as autografts or allografts, require harvesting bone from the patient's own body or from a donor, which can be a painful and invasive procedure with potential complications. In contrast, an embodiment of the present disclosure may be fabricated from readily available materials and does not require harvesting bone tissue from the patient or a donor. Further, embodiments may be fabricated in a short time period, such as, for example, in two days. This fabrication process is faster than the fabrication process of other bone grafts that require the harvesting of bone tissue from the patient or a donor.

Embodiments of the disclosure herein provide for structures that are 3D printable, load supporting, and capable of extreme mechanical and geometrical tenability. The bone graft scaffolds disclosed herein provide for low immunogenicity and biodegradability, which makes the disclosed bone graft scaffold a suitable option for use in oncological applications where traditional bone grafts may not be suitable. The disclosed bone graft scaffolds do not cause exothermic reactions.

Embodiments of the present disclosure provide a bone graft scaffold having a lattice structure. Lattices structures have highly interconnected pores, which provides a large surface area for cell attachment and proliferation. In embodiments of the present disclosure, the lattice structure may have a triply periodic minimal surface shape (TPMS). TPMS shapes may include a gyroid lattice, a diamond, an I-WP, a neovius, a primitive, a Fischer-Koch S, an F-RD, or a PMY. For example, a gyroid lattice structure may be used to create patient-specific bone graft scaffolds for complex bone defects.

Embodiments of the lattice structure may be 3D printed. Further, embodiments of the lattice structure as disclosed herein may be made of any materials, such as ceramic materials or plastics. Ceramic materials may include inorganic composites including calcium, phosphorus, silicon dioxides, and/or calcium carbonates. In embodiments, materials that are compatible with 3D printing techniques may be used.

Embodiments of the present disclosure include lattice structures made of minerals (or mineralized lattices). A base of monomers and/or photoinitiators may be combined with minerals, such as ceramic particles to form a printable resin, such as the lattice structure. In an embodiment, calcium-based minerals may be used. For example, hydroxyapatite (Ca₁₀(PO₄)6(OH)), Wollastonite (CaSiO₃), and/or β-TCP (Ca₃(PO₄)) may be used alone or in combination with one another or other materials. Embodiments of the present disclosure may include a 3D printed lattice structure, such as an Osteolite® lattice structure, that is patient specific and has a high strength. In embodiments, the lattice structure may not be made of minerals (not mineralized). In embodiments, the lattice structure may be made of plastic materials.

In an embodiment, the lattice structure as disclosed herein may be sintered in order to burn off cytotoxic organic components of fabricated lattices (e.g., 400° C.) and/or to induce physical change that decreases pore size and improves mechanical properties (e.g., >1000° C.). The lattice structure may go through a series of heating and sintering processes. Mineralized and un-mineralized lattices may be sintered. Embodiments of the present disclosure further include unsintered lattices.

In an embodiment, the lattice structure may be tuned to match the mechanical properties of the surrounding tissue, promoting cell differentiation and implant integration. For example, the material strength, lattice pore size, cryogel integration potential, etc., may be tuned. Further, geometric properties such as strut thickness, unit cell size, unit cell shape, etc., may be tuned. In an embodiment, the lattice structure may have a solid volume from 0.5 to 150 cm³, a relative density from 10% to 50%, and/or a pore diameter from 0.5 to 5 mm. The lattice structure and dimensions allow for easy integration with existing bone tissue, which is an advantage over some synthetic bone fillers and cements. The lattice structure shape may also provide anisotropic mechanical properties, which may be used to match the directional alignment of the surrounding native tissue.

Embodiments of the present disclosure further include a cryogel. A solution made of two polymers cross-linked together, may be frozen and dried out to form a cryogel, a sponge-like material with interconnected pores. The cryogels may be made of biocompatible materials such as silk, collagen, polyacrylamide, alginate, cellulose, chitosan, gelatin, laminin, fibrinogen, polyethylene glycol (PEG), polyethylene oxide (PEO), polyvinyl alcohol (PVA), and/or N-Vinylpyrrolidone (NVP). Chitosan and gelatin are FDA approved for use in humans. For example, the formulated cryogel solution may be made of chitosan-gelatin solution. The chitosan-gelatin cryogel may be highly macroporous and can promote cellular adhesion and cell proliferation. In other embodiments, the cryogel may be made of MXenes. The MXene cryogel may enhance osteogenic differentiation when electrically stimulated in irradiated bone. Embodiments of the MXene (MX) cryogel may include any percentage of MX, for example 30%, MX, 50% MX, or 70% MX. 70% MX cryogels. Embodiments of the present disclosure include a cryogel solution that has a porosity from 50% to 80%.

Even further, embodiments of the present disclosure may include a composite bone graft scaffold including the lattice structure and the cryogel. Any combination of lattice structures and cryogels disclosed, that allow for the integration of the two materials, may form the composite bone graft scaffold. For example, to fabricate embodiments of the bone graft scaffold disclosed herein, the cryogel solution must be able to mix and pour over the lattice structure. If the unit cell density becomes too high, due to small pore size or thick struts, it may be too difficult to mix the viscous cryogel solution with the lattice structure, and the bone graft scaffold may not be formed. Any combination that allows for the cryogel solution to be pushed through the lattice structure to integrate the two materials is disclosed herein.

By combining cryogels with lattice structures, the present disclosure may provide a customizable bone graft scaffold that can be tailored to the specific needs of individual patients. The shape of the bone graft scaffold may be dependent on the patient's bone defect and may be formed to fit the patient's bone defect. For example, the discrete geometry of the overall shape of the bone graft scaffold and lattice density parameters may be customized. Strut thickness, unit cell size, and unit cell shape of the lattice structure may be customized. This is particularly useful for complex bone defects where multiple grafts or repeat surgeries are required, and where the limited supply of donor tissue may be a challenge.

In an embodiment, the bone graft scaffold may have a porosity from 25% to 70%, a relative swelling ratio from 1500% to 2500%, and/or a mechanical strength from 0.5 MPa to 100 MPa.

The present disclosure provides a bone graft scaffold with favorable mechanical properties, pore distribution, swelling capacity, biocompatibility, and biodegradability. When the bone graft scaffold is applied to critical sized bone defects, it may regenerate bone tissue at an accelerated rate with a low immunogenic response, outperforming standard of care bone graft substitutes and bone grafting techniques alike.

Embodiments of the bone graft scaffold described herein have an increased compressive stiffness. In an embodiment, the compressive modulus of the disclosed bone graft scaffold may be 120× larger than the compressive modulus of a cryogel without a lattice structure. The present disclosure is highly elastic when the cryogel is combined with the lattice structure. After 99% ultimate compression, cryogels rebound to and maintain their original shape (shape memory). Further, in an embodiment, the bone graft scaffold may swell by 200%.

The ability to customize the lattice structure and pore size of an embodiment of the present disclosure makes it suitable for a wide range of clinical scenarios, including patient-specific needs resulting from injuries related to segmental defects in long bones such as the femur, humorous, or tibia, either acute, from tumor resection, infection, or big fractures, or from craniofacial defects requiring plastic reconstruction. The size of the bone graft by volume may vary from 0.5-150 cm³ and may be dependent on the bone location and the type of reconstruction required.

An embodiment of the present disclosure may be used in indications that require load support (bearing) and no setting time (ready for use and implantation immediately after unpackaging). An embodiment may be used for weight-bearing applications.

An embodiment of the present disclosure may be used in conjunction with less invasive metallic implants and/or plates and screws to share a load.

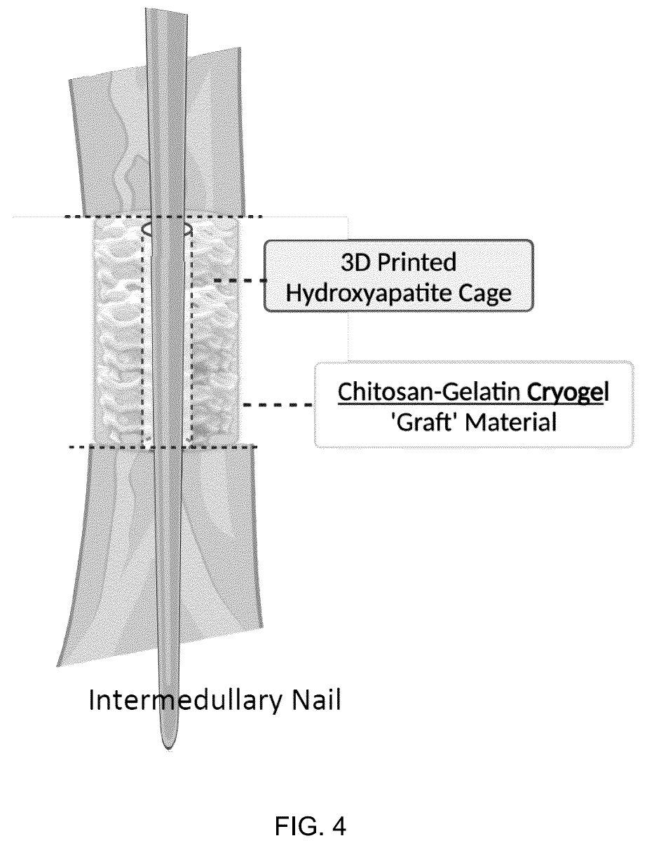

An embodiment of the present disclosure may be used in orthopedic reconstruction for patients following a bone tumor resection. In particular, embodiments may be used in/on patients suffering from primary bone sarcomas and metastatic bone disease. These patients are typically treated by removing the cancerous tissue, which leaves a critical sized bone defect that will not regenerate on its own. Most of the time, these patients then undergo invasive reconstructive surgeries, where the missing bone and some of their healthy bone tissue is replaced by large metallic implants (endoprosthesis). The present disclosure may be used to fill the critical sized defects to augment the reconstruction and repair of these patients by providing a relevant bone graft scaffold for their natural, healthy bone to grow though. An embodiment of the present disclosure will work to augment the reconstruction process by promoting bone ingrowth with less invasive metallic fixation techniques instead of replacing most of their healthy bone with prosthetic implants entirely. For example, FIG. 4 displays an embodiment of the present disclosure filling a segmental defect.

Embodiments of the present disclosure may be biodegradable, made of FDA approved materials, have a tailorable geometry, have low immunogenicity or be non-immunogenic, have a bone mimicking pore network, be bioactive, and promote regeneration.

The application of the present disclosure may provide an improvement in the quality of life of a patient, less surgery time and frequency, and less time at hospital. For example, embodiments of the present disclosure may decrease the likelihood of infection and promote natural bone healing, for example, while in the presence of radiation.

The following examples are presented to illustrate the present disclosure. They are not intended to be limiting in any matter.

### Example 1

This example provides a description of an embodiment of the bone graft scaffold (or composite bone graft sample).

Cryogels, known for their biocompatibility and porous structure, lack mechanical strength, while 3D printed scaffolds have excellent mechanical properties but limited porosity resolution. Embodiments of the present disclosure combine a 3D printed plastic gyroid lattice structure with a chitosan-gelatin cryogel to form a composite bone graft scaffold that balances the advantages of both fabrication methods. This example compares the pore diameter, swelling potential, mechanical characteristics, and cellular infiltration capability of composite bone graft scaffolds and control cryogels. The incorporation of the 3D printed lattice demonstrated patient-specific geometry capabilities and significantly improved mechanical strength compared to the control cryogel. The composite bone graft scaffolds exhibited similar porosity and relative swelling ratio to the control cryogels. This example presents an approach to combine two scaffold types to retain the advantages of each scaffold type while mitigating their shortcomings.

This section of Example 1 describes the materials and methods of an embodiment disclosed herein.

### Gyroid Lattice Design

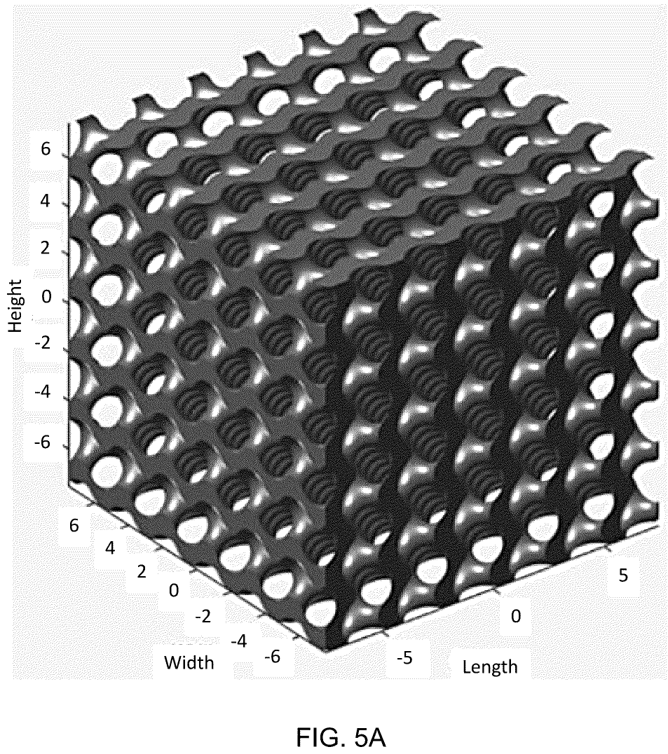

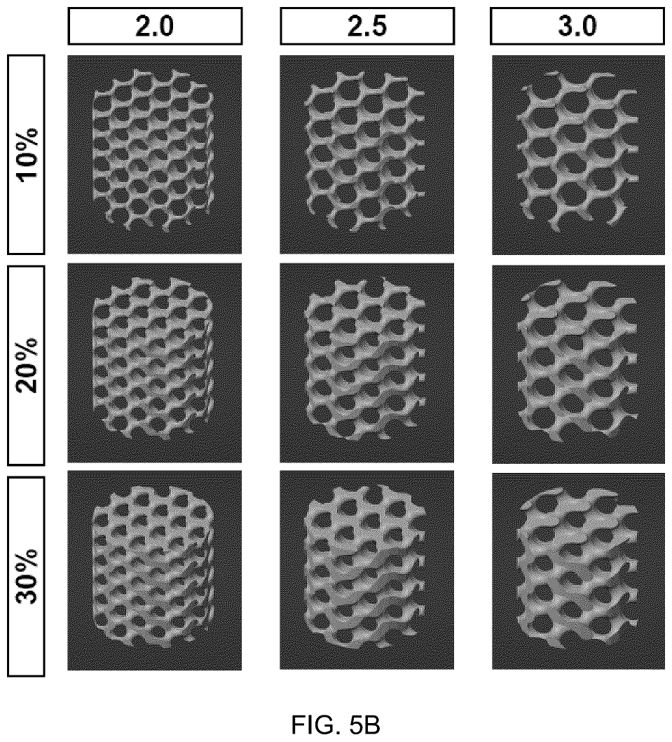

In this example, MSLattice was used to generate a solid cuboid gyroid lattice structure, shown in FIG. 5A. The program accepts six parameters: width, length, height of the sample (in unit cells), unit cell size (dimensionless), relative gyroid density of the lattice structure (0-100%), and mesh density points. The relative density may be 10-50%, and the unit cell sizes, or cell pore diameter, may be 0.5-5 mm. In an embodiment, the generated gyroid lattice structures used 10%, 20%, and 30% relative densities and 2.0, 2.5, and 3.0-unit cell sizes, as shown in FIG. 5B. Mesh density points were kept constant at 50, and sample length, width, and height were set to an arbitrary value to generate a larger gyroid than what was needed in every dimension.

The cuboid gyroid STL files generated by MSLattice were then imported into Blender (Blender Institute; Amsterdam, Netherlands) using a unit conversion of 1 unit=1 mm, where a Boolean intersection was performed between them and a cylinder (10 mm height by 9 mm diameter). Unconnected mesh artifacts from this procedure were removed within Blender. The cylinders were then scaled down to 8.9 mm diameter to fit within the syringes. Nine lattice structures were generated with varying solidity (10%, 20%, 30%) and pore size (2.0, 2.5, 3.0 mm) (FIG. 5B).

Relative density refers to the solid volume fraction of the lattice structure which is defined as the ratio of the solid volume of the lattice structure to the volume of the space that the lattice structure occupies. In this example, relative density will be referred to as solidity because this is a more intuitive way to describe the 3D printed lattices. Higher solidity lattice structures use more material to manufacture and possess thicker branches.

FIG. 5A shows the MSLattice interface with generated cuboidal gyroid and FIG. 5B shows 3D renderings of gyroid cylinder samples (later referred to as 3D printed lattice structures). Columns represent different gyroid pore sizes and rows represent different gyroid solidity (fill percentage).

### 3D Printing Gyroid Lattice Structures

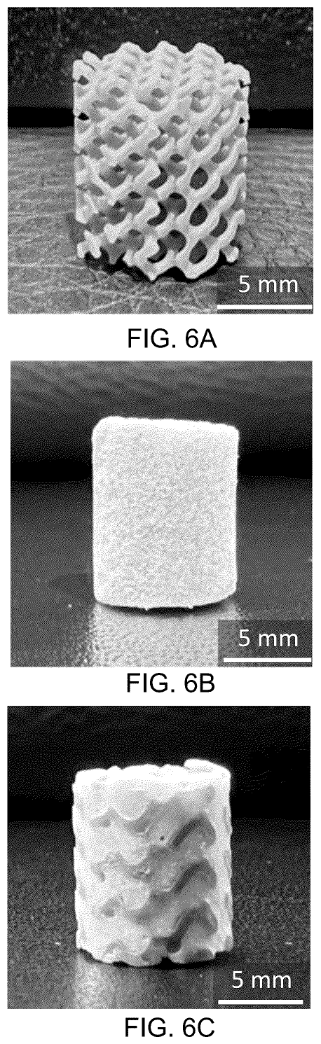

The 3D printed lattice structures were printed on a Form 3B SLA 3D printer (Formlabs, Massachusetts) using Grey V4 Resin (Formlabs, Massachusetts) and Preform 3D print preparation software (Formlabs, Massachusetts). The lattice structures were printed using a 0.160 mm layer height and Preform's Default (v1.1) print settings. The lattice structures were printed as cylinders with heights of 10 mm and diameters of 9 mm, as shown in FIG. 6A.

### Preparation of Chitosan—Gelatin (CG) Cryogel Solution

CG cryogels were made with reference to previously described methods. An aliquot of 10 mL of 1% acetic acid (Fisher Scientific, New Jersey) in deionized water (DI) was prepared. This solution was then split into 8 and 2 mL scintillation vials. Low viscosity chitosan (80 mg; Mw=1526.464 g/mol; MP Biomedicals, Ohio) was added to the 8 mL aliquot and vortexed for 30 seconds before placing it on a mechanical spinner for 1 hour. Gelatin from cold water fish skin (320 mg; Mw=60 kDa; Sigma-Aldrich, Missouri) was then added to the 8 mL aliquot and placed on a mechanical spinner for 1 hour, ensuring the gelatin was completely dissolved. The remaining 1% acetic acid 2 mL vial was combined with glutaraldehyde (Sigma-Aldrich, Missouri) to create a 1% glutaraldehyde solution. The 8 mL and 2 mL scintillation vials were then placed in a 4° C. fridge for 1 hour.

### Composite Bone Graft Scaffold Integration

3 mL syringes (Fisher Scientific, New Jersey) containing two gyroid lattice structures each were pre-frozen at −23° C. for 6 hours. The 8 mL and 2 mL cryogel solutions described previously were then mixed by decanting between the vials and immediately poured into the syringes. After the syringes were filled, the plunger was inserted, and the syringe flipped upside down. The plunger was depressed until the cryogel solution completely infiltrated the entire gyroid lattice structure, allowing for some cryogel solution to be expelled from the syringe if necessary. Immediately following this, the syringes were placed in a −23° C. freezer for 18 hours to crosslink at subzero temperatures, following a thawing process within the syringe. FIG. 6C shows the lyophilized composite bone graft scaffold. Plain cryogels served as control and will be referred to as control cryogels, as shown in FIG. 6B.

### Pore Analysis

Scanning electron microscopy (SEM; VEGA3; TESCAN, Czech Republic) was used to observe pore structure on embodiments of the cryogels. Composite bone graft scaffolds were frozen at −80° C. for 1 hour prior to being lyophilized (FreeZone Freeze Dryer, Labconco, Missouri) overnight. The samples were then mounted on an aluminum stub and sputter coated (HUMMER 6.2; Anatech, Nevada) for 240 seconds in gold at 15 mA under the pulse setting to avoid overheating. SEM was then used to obtain images at 100×, 200×, 500× and 1000× for all composite bone graft scaffolds.

ImageJ was used to analyze the pore diameter in the composite bone graft scaffolds. First, the line function was selected and was used to determine the scale via the scale bar in the SEM image. The unit of length was adjusted to microns for accurate measurements. Next, the selection tool was used to measure the length of a representative pore, specifically focusing on the long diameter. The measurement was recorded, and the process was repeated 60 times, taking 15 measurements from each quadrant of the image. The data was saved in an Excel file and the length values were used for statistical analysis.

### Swelling Kinetics

To evaluate the shape retention and rehydration potential of composite bone graft scaffolds, a swelling test was performed. Three samples of each scaffold variation were lyophilized for 24 hours prior to recording the dry weight. Each sample was placed in a weigh boat containing 5 mL of deionized (DI) water, removed, and weighed at nine time points: 2, 4, 10, 20, 40 min, 1, 2, 4, and 24 hours. The average swelling ratio, considering the original dry weight of each sample was recorded using the following equation.

$$Swelling ratio = ( {W}_{h} - {W}_{d} ) / {W}_{d}$$

In this equation, Wₕ is the hydrated weight and W_{d} is the dry weight. The final weights subtracted out the weight of the plastic gyroid lattice structure to isolate the swelling that is derived from only the cryogel portion of the composite bone graft scaffold.

### Mechanical Testing

Ultimate compression testing was conducted to 75% for all composite bone graft scaffolds following hydration for 5 minutes in phosphate buffered saline (PBS; Fisher Scientific; New Jersey). Compression was completed using an Intron 68SC-2 system (Instron, Massachusetts) with a 500 N load cell and set parameters of a test rate of 10 mm/min, preload of 0.05 N, and preload speed of 1 mm/min. All data was analyzed through the Bluehill universal software (Instron; Massachusetts) the compressive modulus (MPa) was taken from the software output for the composite bone graft scaffolds and control cryogels.

### Cellular Infiltration

Based on these results, a total of 15 samples (N=3 per time point) of each of the 20% solidity composite bone graft scaffolds were sterilized in 70% ethanol (Fisher Scientific; New Jersey) for 30 min, followed by three, 10 min washes with sterile PBS. The composite bone graft scaffolds were then placed in a sterile 24-well plate (Falcon, New York) with 100 L of Dulbecco's Modified Eagle's Medium (DMEM; ThermoFischer Scientific, Massachusetts), 10% fetal bovine serum (Omega Scientific Inc.; California), and 1% penicillin-streptomycin solution (Life Technologies Corporation, California) containing 50,000 human bone osteosarcoma-derived cells (MG-63, passage 97; ATCC, Virginia). The cells were seeded on each scaffold through a dropwise method and left to incubate for two hours at 37° C. and 5% CO₂ to allow for cell attachment. After this time period, an additional 200 μL of complete media was added so that all samples were fully submerged. Media was changed every two to three days from around the scaffold. The composite bone graft scaffolds and media were cultured to day 5, 7, 14, 21, or 28, at which time the composite bone graft scaffolds were placed in formalin (Fisher Scientific; New Jersey) for 24 hours and then stored in PBS.

To prepare the cryoprotectant medium, a 30% sucrose (w/v; Sigma-Aldrich, Missouri) solution in DI was created and thoroughly mixed on a shaker plate. The cryogels, previously stored in PBS, were then submerged in individual 5 ml Eppendorf tubes filled with the cryoprotectant solution. These tubes were placed in a 4° C. refrigerator for 24 hours. After the cryoprotection process, a gelatin-sucrose embedding solution was prepared. A 5% gelatin from porcine skin (w/w; Sigma-Aldrich, Missouri)—5% sucrose (w/w; Sigma-Aldrich, Missouri) solution in DI water was prepared and dissolved using a water bath. Embedding molds were retrieved and appropriately labeled. After the embedding solution was fully prepared, 1 mL of the solution was added to each mold and the cryoprotected samples were transferred to their respective molds. An additional embedding solution was added to ensure complete coverage of the scaffolds. The molds were then placed in a 45° C. oven and incubated for 2 hours. The samples were then transferred to a −80° C. freezer and allowed to freeze overnight. Cryosectioning (Cryostat Microm HM 525; Thermo Scientific, ThermoFischer Scientific, Massachusetts) was performed at a thickness of 20 μm, and the sections were stained with 4′,6-diamidino-2-phenylindole (DAPI; BD Biosciences, California) to assess cellular characteristics. Images were taken by an optical light microscope (Laxco, Washington) at 100×.

### Statistics

GraphPad Prism was utilized to conduct all statistical analyses, employing a significance level of 0.05. A two-way ANOVA, followed by a Tukey post hoc analysis, was performed to assess the significance among different groups. The presence of outliers in the swelling ratio was determined using the ROUT method, with a Q value of 1%.

This section of Example 1 describes the results of the embodiment disclosed herein as compared to the control, described in the materials and methods section.

### Pore Analysis

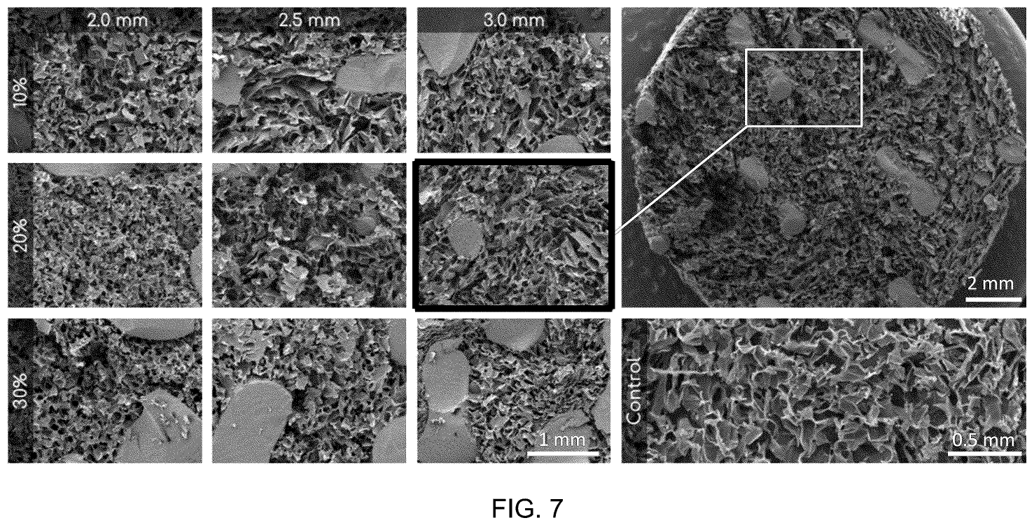

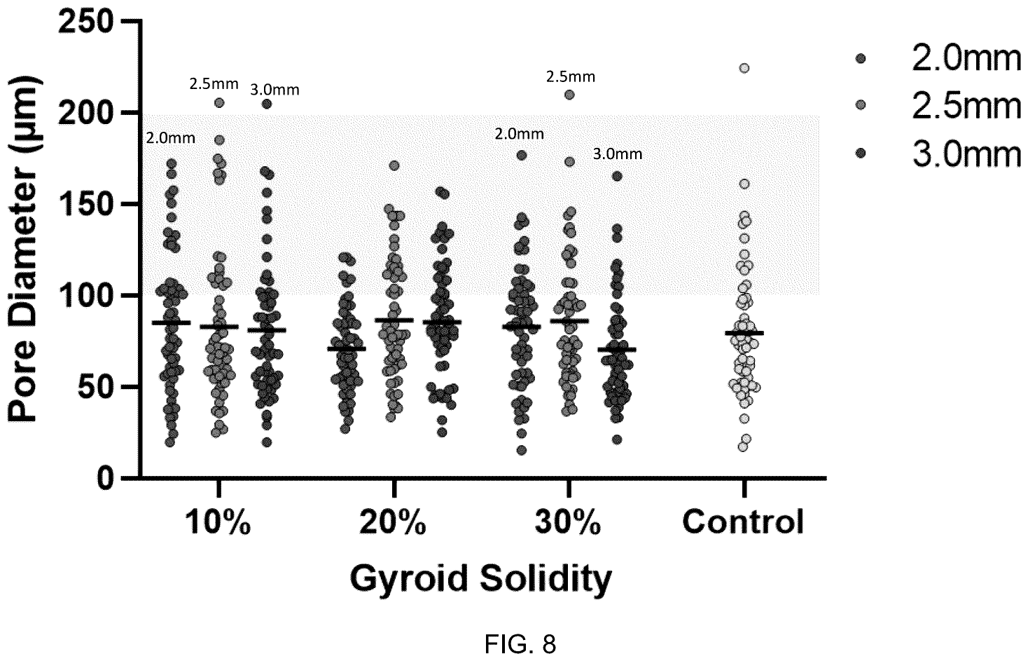

SEM was used to capture images of the composite bone graft scaffolds, as shown in FIG. 7. Specifically, FIG. 7 displays SEM images of composite bone graft scaffolds and control cryogel (bottom right). The pore areas for each scaffold were calculated using ImageJ. The resulting average and standard deviations (std. dev.) for the nine types of composite bone graft scaffolds are presented in Table 1. The distribution of pores can be seen in FIG. 8. As shown, the solid lines are mean values, and the orange highlight is the range for ideal pore size (100-200 μm). There were no significant differences between any of the scaffolds or the control.

TABLE 1
Averages and standard deviations for cryogel pore size inside
the composite bone graft scaffolds (n = 60). Control
mean is 79.27 μm and standard deviation (std. dev.) is 35.86 μm.

| | 10% Solidity | | | 20% Solidity | | | 30% Solidity | | |
|---|---|---|---|---|---|---|---|---|---|
| Gyroid Pore Size (mm) | 2.0 | 2.5 | 3.0 | 2.0 | 2.5 | 3.0 | 2.0 | 2.5 | 3.0 |
| Mean (μm) | 84.90 | 82.85 | 80.90 | 70.82 | 86.27 | 85.38 | 82.73 | 86.01 | 70.22 |
| Std. Dev. (μm) | 37.74 | 41.70 | 37.41 | 23.13 | 32.66 | 31.09 | 32.83 | 34.84 | 28.35 |

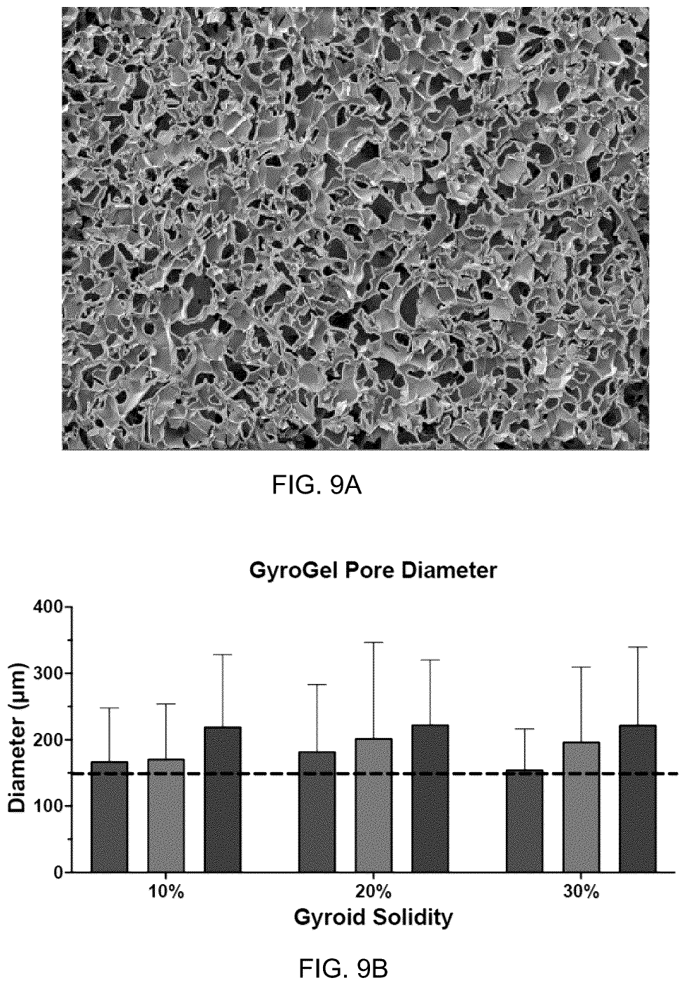

FIG. 9A displays the output of a python generated pore analysis program. FIG. 9B displays results of a pore analysis completed. The dotted line represents the control value. As shown, there is a statistical different between the pore diameters of the 30% 2.0 mm and 30% 3.0 mm of the composite bone graft scaffold (P<0.05). Through these results, it was found that increasing 3D printed infill unit size increased the pore diameter of the cryogel.

### Swelling Kinetics

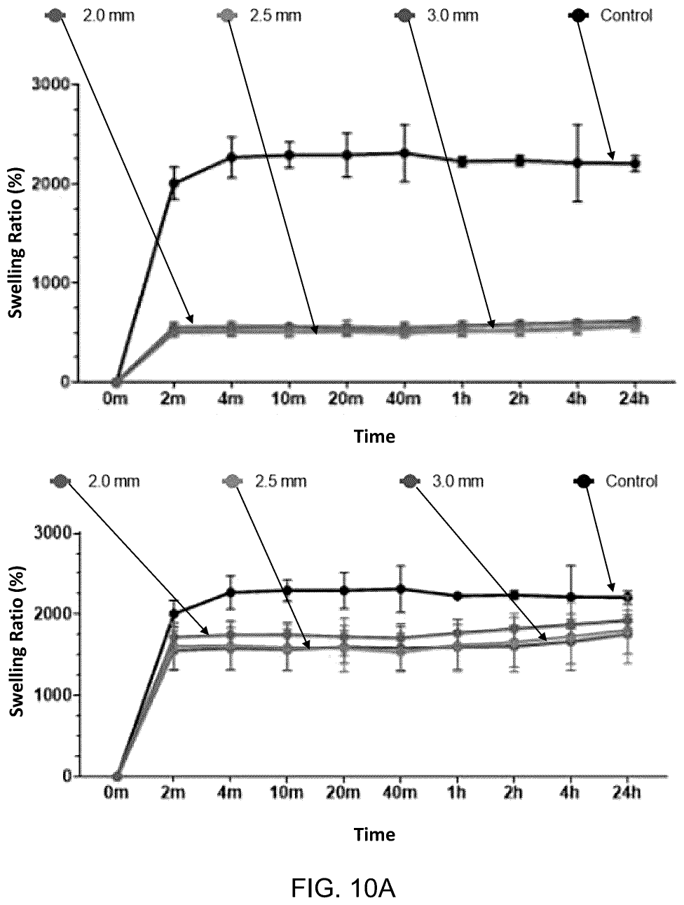

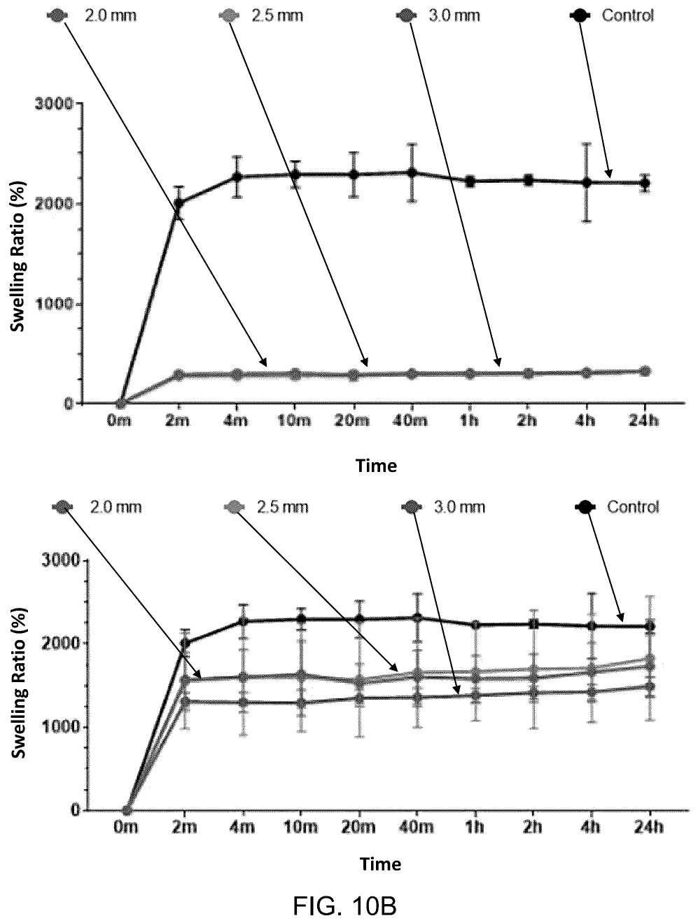

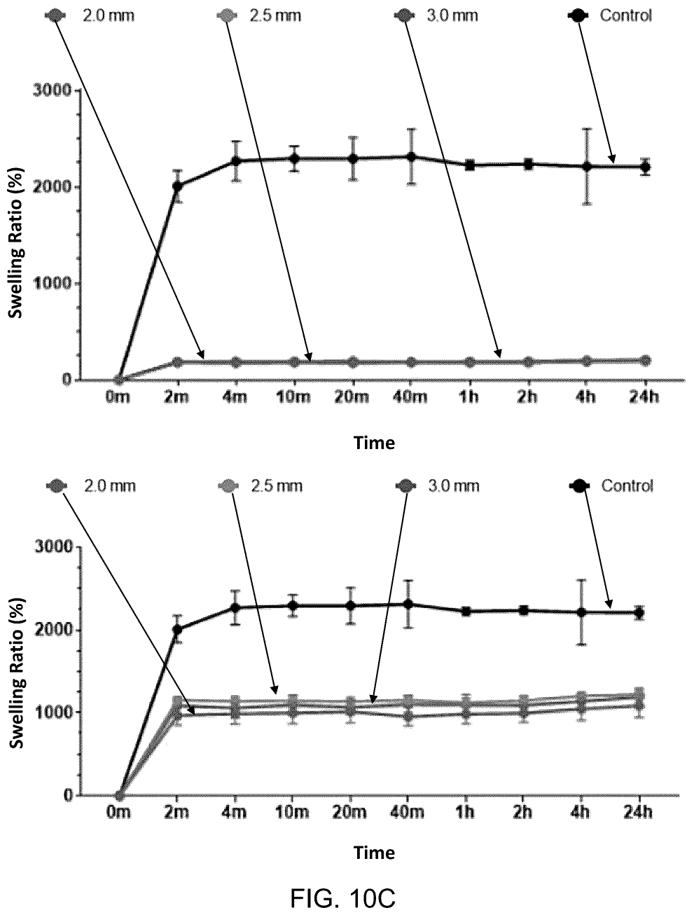

Following dehydration and subsequent immersion in water, all composite bone graft scaffolds achieved maximum swelling capacity in 2 minutes (FIGS. 10A-10C). Specifically, FIGS. 10A-10C display (FIG. 10A) 10%, (FIG. 10B) 20%, (FIG. 10C) 30% solidity composite scaffold absolute swelling ratios (left column) and relative swelling ratios (right column). Relative swelling ratios were with 3D printed structure weights subtracted from total scaffold weight.

To accurately assess the swelling ratio of the cryogel within the composite bone graft scaffold, the weight of the plastic lattice was subtracted from the overall weight of the composite bone graft scaffold. This ensured that only the weight of the cryogel was considered in each final measurement. Post-processing analysis revealed that the 10%, 20%, and 30% solidity composite bone graft scaffolds swelled to approximately 1800-1900%, 1500-1800%, and 1000-1200% of their respective dry weights. There was no significant difference in swelling ratio for the control cryogel after the 4-minute mark and no significant difference in swelling ratio after the 2-minute mark for each composite bone graft scaffold (Q=1%). However, the swelling ratios of the 30% solidity composite bone graft scaffolds were significantly different from the control group at all of the time points. Additionally, the swelling ratios of the 10% 2.0 mm composite bone graft scaffolds exhibited significant differences when compared to the 10% 3.0 mm composite bone graft scaffolds at all of the time points, except for the 20-minute mark (P<0.05). Through these results, it was found that increasing 3D-printed infill percentage decreased the swelling ratio of the cryogel.

### Mechanical Testing

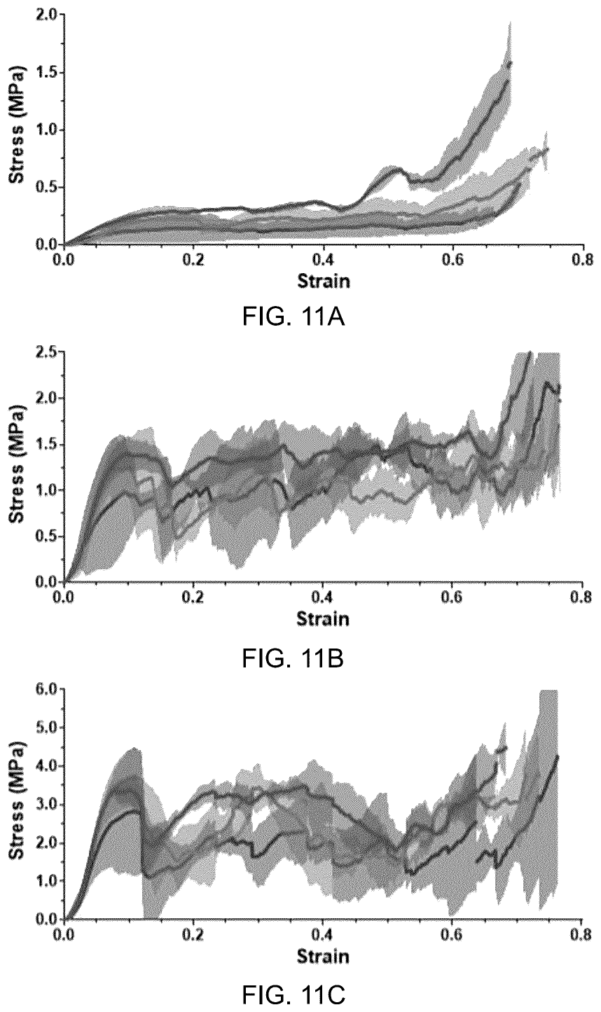

The ultimate compression of the scaffolds was assessed at 75% strain. The stress versus strain graphs for the 10% solidity composite bone graft scaffolds exhibited a steady increase, whereas the stress versus strain graphs for the 20% solidity and 30% solidity composite bone graft scaffolds exhibited multiple peaks (FIGS. 11A-11C). The 30% solidity composite bone graft scaffolds demonstrated the highest compressive moduli, followed by the 20% solidity and 10% solidity bone graft scaffolds, respectively. The average young's modulus of the control was 0.032 MPa with a standard deviation of 0.0029 MPa. The solidity of each composite bone graft scaffold was significantly different from that of the control group (P<0.05). Furthermore, there were significant differences in the compressive modulus between the bone graft scaffolds with 10% solidity and the bone graft scaffolds with 30% solidity (P<0.05). Similarly, the compressive modulus of the bone graft scaffolds with 20% solidity differed significantly from that of the bone graft scaffolds with 30% solidity (P<0.05). The compressive moduli for each composite bone graft scaffold are presented in Table 2.

TABLE 2
Compressive modulus for each composite bone graft scaffold

| | 10% Solidity | | | 20% Solidity | | | 30% Solidity | | |
|---|---|---|---|---|---|---|---|---|---|
| Gyroid Pore Size (mm) | 2.0 | 2.5 | 3.0 | 2.0 | 2.5 | 3.0 | 2.0 | 2.5 | 3.0 |
| Mean (MPa) | 4.23 | 4.28 | 2.50 | 28.33 | 29.62 | 21.60 | 69.36 | 73.69 | 58.29 |
| Std. Dev. (MPa) | 0.66 | 2.34 | 2.26 | 6.04 | 5.62 | 8.08 | 2.67 | 15.16 | 32.24 |

### Cellular Infiltration

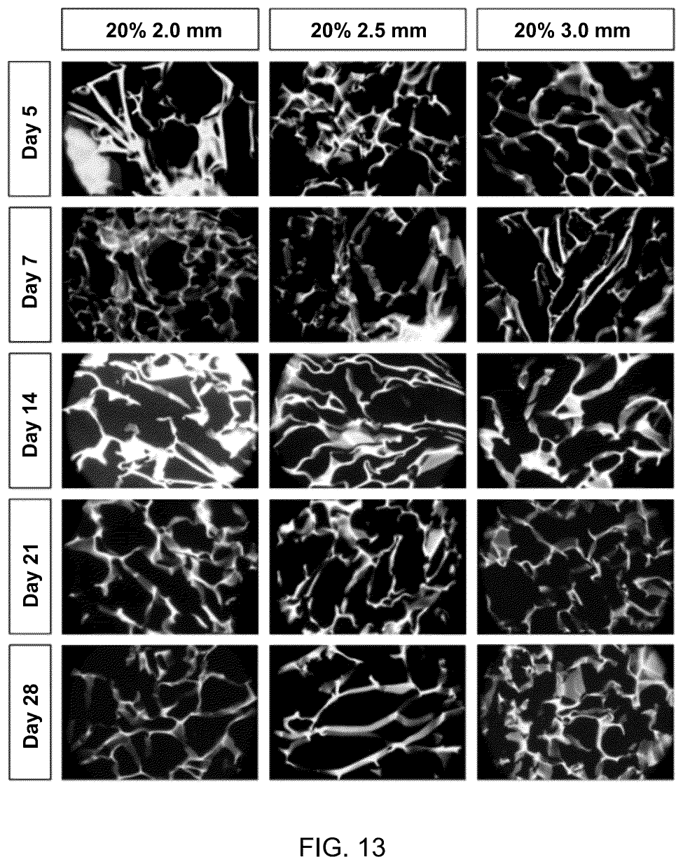

All composite bone graft scaffolds were seeded with MG-63 osteosarcoma cells and incubated to support infiltration over 28 days. Cryosectioning was used to obtain cross-sectional samples from five different regions within each scaffold to assess the extent of cellular infiltration (FIG. 13). Specifically, FIG. 13 displays cell infiltration-stained images using DAPI staining and light microscopy. The individual fluorescent blue dots indicate the nuclei of the MG-63 cells. The green-yellow structures indicate the cryogel scaffold. The large fluorescence indicated the 3D printed lattice (e.g., left side of the day 5 20% 2.0 mm composite bone graft scaffold).

The objective of Example 1 was to assess the advantages of incorporating a 3D printed lattice into a cryogel. Nine distinct 3D printed lattice structures were fabricated by varying the pore size (2.0, 2.5, 3.0 mm) and solidity (10, 20, 30%) of a gyroid shape. Subsequently, the nine lattice structures were permeated with a cryogel, producing nine unique sample types to assess the composite bone graft scaffold. The composite bone graft scaffolds were evaluated and compared with control cryogel based on porosity, swelling capacity, mechanical integrity, and cell compatibility.

Visual examination of the SEM images in FIG. 7 demonstrates that the incorporation of a 3D printed plastic lattice into the cryogel impacts the pore architecture. Specifically, there appears to be some interaction between the cryogel and lattice material, as evidenced by attachment points between them. SEM analysis indicated that the pores farther from the lattice appeared larger than those in the control cryogel, while those closer to the attachment points were smaller. This pattern may be explained by the plastic's hindrance of ice crystal formation within the cryogel. The ice crystals near the plastic lattice would hypothetically be smaller than the ice crystals further from the lattice. As a result, the smaller ice crystals melt to leave behind smaller pores near the lattice and larger ice crystals melt to produce bigger pores further from the lattice. Despite this, the addition of the plastic lattice did not significantly alter the pore size of the composite bone graft scaffolds, except in the 10% 2.5 mm sample. It can be crucial to maintain the porosity of the cryogel to ensure its successful incorporation into bone defect sites, because pore size and interconnectivity are typically essential for cell attachment and the transport of nutrients and growth factors. Previous examples suggest that a pore diameter of 100-200 μm is optimal for bone regeneration. Although the mean pore sizes in this study were below the optimal range, they remained similar to the control cryogel. It is hypothesized that the pore size for all samples shrunk due to lyophilization because cellular infiltration was achieved. It is also possible that the hydrated form of the bone graft scaffold could have larger pores. The pore size in its hydrated state may also be analyzed using microCT.

The ability to rapidly swell is an advantageous property for scaffolds in bone tissue engineering applications. High swelling capacity allows cryogels to quickly fill bone defect sites without the need for pre-wetting, facilitating nutrient absorption and promoting even cell distribution for enhanced regrowth at wound sites. In this example, all cryogels achieved maximum swelling capacity after four minutes. However, incorporation of a 3D printed lattice structures with 30% solidity reduced the swelling capacity compared to control cryogel. While the 10% and 20% solidity composite bone graft scaffolds exhibited no significant difference in swelling capacity compared to control cryogel, their absolute swelling capacity was also lower (FIGS. 10A-10C). Therefore, the presence of a 3D printed lattice structure that does not swell may hinder nutrient absorption and decrease the number of infiltrating cells in the overall scaffold in vivo. However, a scaffold with a 3D printed lattice structure is still improved compared to previous options and has other benefits.

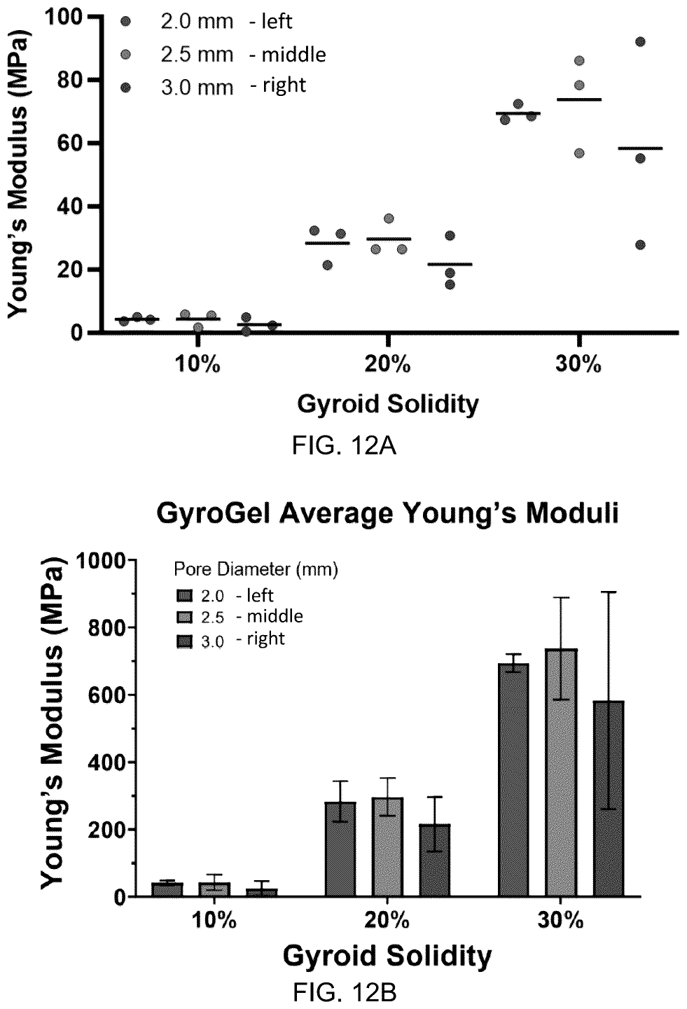

Cryogels possess a highly porous, sponge-like structure with elastic properties, allowing them to be compressed to 90% and rebound to their original shape without experiencing crack propagation. Compression testing was performed to evaluate the mechanical durability and strength of the composite bone graft scaffolds. The stress-strain curves for the 10% solidity composite bone graft scaffolds showed a gradual increase in slope, indicating a lack of abrupt scaffold fracture with increasing strain (FIGS. 11A-11C). Conversely, the stress-strain curves for the 20% and 30% solidity composite bone graft scaffolds exhibited multiple peaks, corresponding to different lattice layers breaking under high loads (FIGS. 11A-11C). Findings show that increasing lattice structure solidity resulted in an increased compressive modulus for the composite bone graft scaffolds, likely due to the greater proportion of plastic in the composite bone graft scaffold resulting in an improved ability to resist compression (FIG. 12A). Notably, the 2.5 mm lattices exhibited the highest mechanical durability of all lattice structure solidities, potentially because of a balance between the stiffness from the plastic and elasticity from the cryogel. It was found that as solidity increased, variability of compressive modulus increased (FIG. 12A). This may be due to a greater amount of cryogel in the lower solidity lattice structures compressing more elastically, whereas the higher solidity had more plastic which had more random fracture patterns. Specifically, FIG. 12A displays the average compressive modulus calculated from Instron 68SC-2 compression test. The control young's modulus is 0.032 MPa. These results suggest that composite bone graft scaffolds possess improved mechanical properties compared to control cryogels, supporting their use in bone defects that may experience greater relative loading.

FIG. 12B displays the average Young's modulus calculated from an Instron 5544 compression test. As shown, each composite bone graft scaffold solidity was significantly different from every other solidity at P<0.05. The Young's modulus of the control was 0.571 MPa. Through these results, it was found that increasing the 3DP infill percentage increased the mechanical strength of embodiments of the composite bone graft scaffold.

For tissue to grow into the cryogel, it can be important for cells to infiltrate and proliferate within it. Only the three 20% solidity composite bone graft scaffolds were used for the cell infiltration study in order to reduce the number of scaffolds that would have to be created to assess all nine lattice structures/scaffold types. The 20% solidity composite bone graft scaffolds were chosen because they possessed favorable properties including similar pore size and relative swell capacity to the control, as well as a medium absolute swell capacity and compressive modulus compared to the 10% and 30% composite bone graft scaffolds. Further, using three different gyroid pore sizes (2.0, 2.5, 3.0 mm) allowed for an investigation in the effect on cell proliferation through the super-macropores of the 3D printed lattice structure portion. In the first five days, MG-63 cells exhibited adherence and proliferation across all three composite bone graft scaffolds, with cells distributed throughout the top layer and penetrating into the middle of the bone graft scaffolds.

Among the different bone graft scaffolds, the 20% 3.0 mm bone graft scaffold exhibited the highest number of cells for the longest period of time (up to day 14). This can be attributed to the larger volume of cryogel it contained, as well as the presence of fewer obstacles that the cells needed to navigate through.

Overall, incorporation of a 3D printed lattice structure within a cryogel did not appear to adversely affect pore formation in the infilled cryogel. The composite bone graft scaffolds were also able to swell to their full potential within four minutes and maintain their size, despite the addition of the plastic gyroid lattice structure. The 10% and 20% composite bone graft scaffolds exhibited a slight decrease in average swelling ratio, while the 30% composite bone graft scaffold exhibited a significant decrease. Additionally, all composite bone graft scaffolds demonstrated significantly lower absolute swelling capacity when compared to the control cryogel. The incorporation of the lattice structure increased the compressive modulus when compared to the control cryogel.

Finally, the 20% solidity composite bone graft scaffolds were seeded with bone osteosarcoma-derived cells, which resulted in a high level of cell infiltration within the first 5 days followed by some cellular death observed in all scaffolds after day 7.

It is desirable to develop an alternative to bone grafts for the treatment of complex critical-size defects. Cryogels are an ideal scaffold for this application due to their macroporous structure and biocompatible properties, although their mechanical strength is relatively weak. In contrast, 3D printed scaffolds offer strong mechanical properties, but current technology often has limited porosity resolution. The bone graft scaffold described herein can combine the advantages of these two scaffold types while minimizing their deficiencies.

This example aimed to achieve this balance by combining a 3D printed plastic gyroid lattice structure with a chitosan-gelatin cryogel. The incorporation of the 3D printed lattice structure led to several benefits over the standard control cryogel, including patient-specific geometry and significant increases in mechanical strength. The composite bone graft scaffolds also exhibited similar porosity and swelling ratio to control cryogels. The 20% solid composite bone graft scaffolds were identified as the most advantageous due to their optimal swelling ratio and mechanical properties. Additionally, the 2.5 mm pore lattice structure was found to possess improved mechanical strength and swelling capacity. However, the range of different lattice structure variables suggests that different parameters could be chosen to apply to different areas of bone such as cortical versus trabecular bone or in different types of loading such as transverse versus longitudinal.

This example demonstrated a method for combining two different types of scaffolds, cryogels and 3D printed lattice structures. In this example, it was found that the presence of the gyroid lattice structures in the cryogels did not interfere with pore shape, increasing the 3D printed gyroid infill increased the cryogel pore diameter and area. Increasing the infill percentage contributed to decreased cryogel swelling potential. Fluctuations in the stress-strain curves represented the layer-by-layer breakage of the bone graft scaffolds during compression. A pore size of 2.5 mm for each of the three infill percentages had the highest mechanical durability.

Different 3D printing materials may also be used for improved biocompatibility and bone cell differentiation.

### Example 2

This example provides a description of an embodiment of a bone graft scaffold, such as a combined biopolymer bone graft scaffold (or, composite bone graft scaffold, or bone graft scaffold) for use in bone regeneration at critical sized defects.

A critical bone defect is a gap in a bone that is around 2.5× larger than the diameter of the tissue. In general, a defect this size to the bone will not heal on its own and will require surgical intervention to regain mechanical structure and function. Bone defects are most commonly the result of acute, high-energy trauma, bone tumor resection, bone infection or necrosis. In general, limb salvaging surgical intervention involves the replacement and reconstruction of the missing bone with aggressive use of metallic implants (intramedullary devices (IM), open reduction and internal fixation (ORIF), or endoprosthesis) in combination with a bone graft of some kind. However, surgical reconstruction remains a challenge with complications rates for bone grafts (auto and allograft), bone transport and fibular graft at 26%, 62%, and 38% respectively. The approaches disclosed in this example aim to provide a biocompatible and mechanically stable environment for bone tissue regeneration, while also promoting the differentiation and proliferation of bone cells with little risk of a negative immune response.

Tissue engineering is a rapidly growing field that combines biomaterial scaffolds, cells, and bioactive factors to repair, replace, or regenerate tissue. Therefore, tissue-engineered scaffolds provide an effective alternative solution to bone grafts, with the potential to promote robust cellular growth, proliferation, and integration at bone interfaces. Many scaffold fabrication techniques have been developed to specifically target the regeneration of bone, including cryogels. Cryogel scaffolds consist of polymer solutions cross-linked at subzero temperatures, where thawing results in a sponge-like, macroporous structure. This durable and porous framework renders these scaffolds optimal for bone regeneration applications.

Having demonstrated the effectiveness of cryogel as a cellular scaffold, this example considers the combination of cryogels with a bone mineral cage, such as gyroid lattice structures. These lattice structures may be 3D printed to fit patient specific geometries, and may provide a rigid, osteoconductive framework that will house the cryogel. Gyroid lattice structures are interconnected macroporous structures whose structure and mechanical properties can be finely tuned. Additionally, its shape and large surface area may provide a favorable microenvironment for cell attachment and growth.

This example investigates the mechanical, morphological, and swelling properties of a composite biomaterial bone graft scaffold, as they pertain to bone regeneration at critical defect sites. Additionally, initial biocompatibility with osteoblast like cells will be qualitatively tested. The composite bone graft scaffold as disclosed herein is a standard chitosan-gelatin biopolymer graft integrated directly with a 3D printed hydroxyapatite lattice structure. While both cryogels and hydroxyapatite have been separately characterized and studied rigorously, the distinct integration of the two has not been studied before. By 3D printing standard gyroid lattice structures (backbones) with a hydroxyapatite resin and pouring chitosan-gelatin solution within the structure whereby the two materials undergo the cryogellation process together, the resultant composite biomaterial bone graft scaffold may exhibit increased compressive stiffness, decreased swelling capacity, unchanged pore sizes, and decreased cellular proliferation and adhesion as compared to a control cryogel. Combining cryogel scaffolds with a 3D printed mineral lattice structure may provide a more mechanically stable, macroporous structure that can be used to create patient-specific bone graft substitute for filling complex bone defects.

This section of Example 2 discusses the methods and materials used in the example described herein.

One of the primary outcomes of this example included the in vitro characterization of the effect of combining cryogel scaffolding (cryogel solution) with a gyroid mineral lattice structure.

Chitosan/gelatin (1:4) in 1% acetic acid solution was mixed and stored in a 4° C. refrigerator. Gyroid lattice structures were designed (Fusion 360 Product Design and Blender) and printed on a DLP 3D printer (Tethon) using Osteolite® (Tethon hydroxyapatite resin). Then, the chilled cryogel solution was poured into 3 cc syringes containing two gyroid lattices each, and immediately placed into a −23° C. freezer for 18 hours to crosslink. Plain cryogels served as the control. After 18 hours combined cryogellation, the composite bone graft scaffolds were removed and underwent a final freeze-dry process (lyophilization). Scanning electron microscopy was used to observe pore structure and pore size ranges of the cryogels (n=1). Elemental dispersion spectroscopy was used to determine the elemental composition of the composite bone graft scaffold (n=1). Swell tests were performed to evaluate the shape retention and rehydration potential of the composite bone graft scaffolds (n=3). Each sample was placed in a weigh boat containing DI water, then removed and weighed at 9 time points: 2, 4, 10, 20, 40 min, 1, 2, 4, and 24 h. The average swelling ratio was determined using the following equation.

$$Swelling ratio = ( {W}_{h} - {W}_{d} ) / {W}_{d}$$

In this equation, Wₕ is the hydrated weight and W_{d} is the dry weight. Compression testing was conducted at 50% compressive strain for all cryogels following hydration for 15 minutes in phosphate buffered saline (PBS) (n=3). Each composite bone graft scaffold was formed in the shape of a cylinder with a diameter and thickness of approximately 10 mm and 8 mm, respectively.

In vitro characterization was performed regarding the effect that the bone graft scaffold would have on initial cellular adhesion and proliferation after 6 days of incubation. To do so, the bone graft scaffolds were sterilized in 70% ethanol followed by three 10-minute washes with sterile PBS. The bone graft scaffolds were then placed in a sterile 48-well plate with 1.5 ml of DMEM 10% fetal bovine serum and 1% penicillin-streptomycin solution containing 50,000 human bone osteosarcoma-derived cells (MG-63, passage 95). The cells were seeded on each bone graft scaffold through a dropwise method and left to incubate for 6 days at 37° C. and 5% CO₂ (n=3). Media was changed every two days from around the bone graft scaffold. On day 6, bone graft scaffolds were placed in formalin for 24 hours. These bone graft scaffolds were then rinsed with PBS and stained with DAPI to visualize cell nuclei. The bone graft scaffolds were imaged using a Nikon spinning disk confocal microscope.

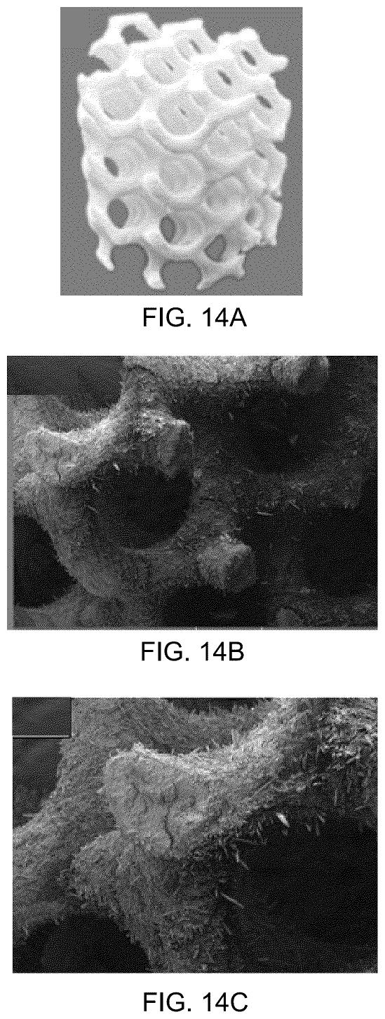

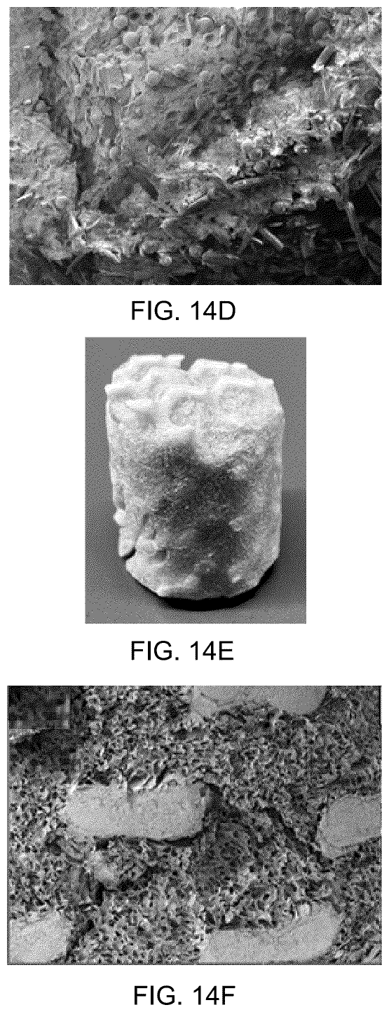

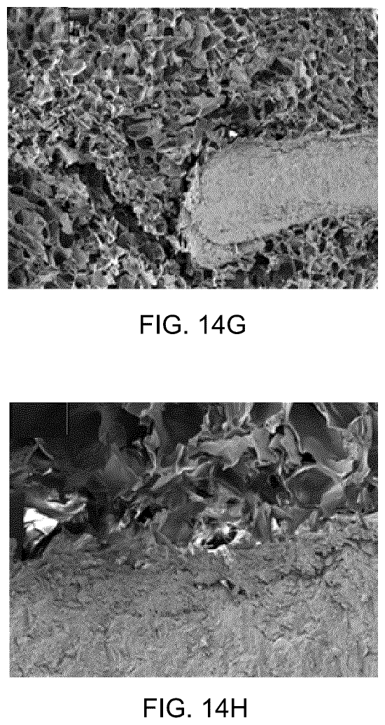

First, the mineral lattice structure was imaged (FIGS. 14A-14D). The lattice structure depicted two unique structural features: crystalline rods (FIG. 14C) and circular beads (FIG. 14D). Although the bead structures and craters may be a result of sputter coating the sample with gold, confocal imaging revealed a similar structure for samples that were not coated (FIG. 14A). Next, the sample's crystalline structure was investigated using X-ray crystallography (XRD). Secondly, the composite bone graft scaffold was imaged (FIGS. 14E-14H), and the scaffolds pores were quantified using a python pore analysis program (FIG. 14G). The average pore diameter of the composite bone graft scaffold was 74.56 μm with a standard deviation of 106 μm, whereas a control cryogel scaffold has an average pore diameter of 71.24 μm with a standard deviation 31.69. Although the pore sizes for the composite bone graft scaffold exhibited greater variance as compared to a control, both scaffold types exhibited a range of pores that fall well within the ideal pore size range of around 100-200 μm.

FIGS. 14A-14H show (FIG. 14A) hydroxyapatite lattice and (FIGS. 14B-14D) SEM images; (FIG. 14E) Composite bone graft scaffold and (FIGS. 14F-14H) SEM images; (FIG. 14G) SEM image with pores counted and measured with the pore analysis program.

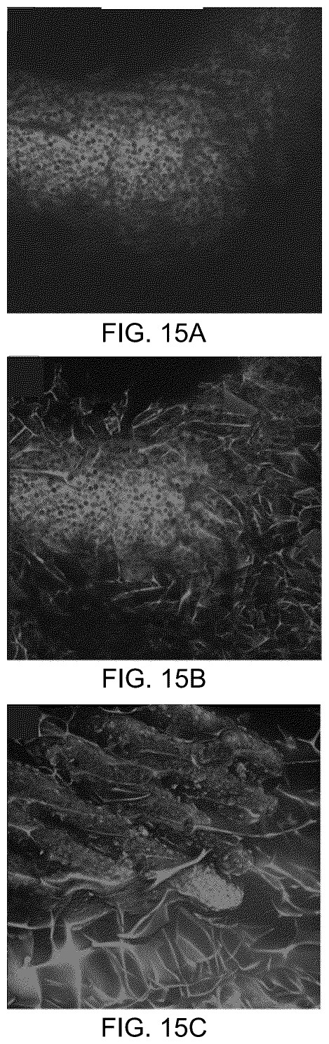

Prior to cellular seeding, composite bone graft scaffolds were imaged using confocal microscopy to investigate its fluorescence and surface features. FIGS. 15A-15C display (FIG. 15A) confocal of mineral lattice fluorescing at 405 nm; (FIG. 15B) both lattice and cryogel imaged at 405 nm and 525 nm (yellow); (FIG. 15C) mineral lattice structure stained with 555 dye.

While previous work has shown that cryogels are auto-florescent at nearly all wavelengths, confocal images (FIGS. 15A-15B) show that the mineral lattice structure itself fluoresces at 405 nm wavelength (blue) without any dye. As shown, the bead-like craters on its surface are also visible in the SEM images. This leads to the conclusion that those surface structures are not caused by gold sputter coating.

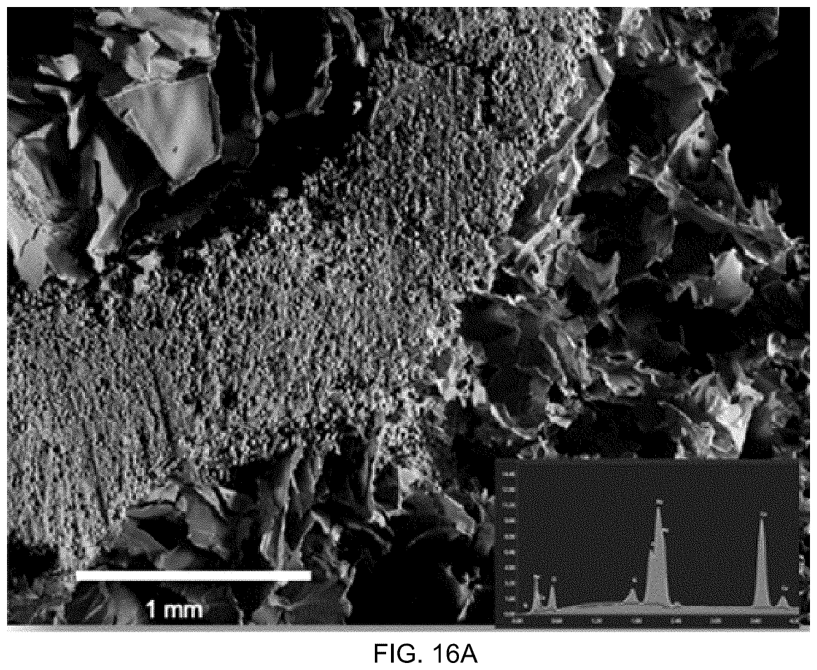

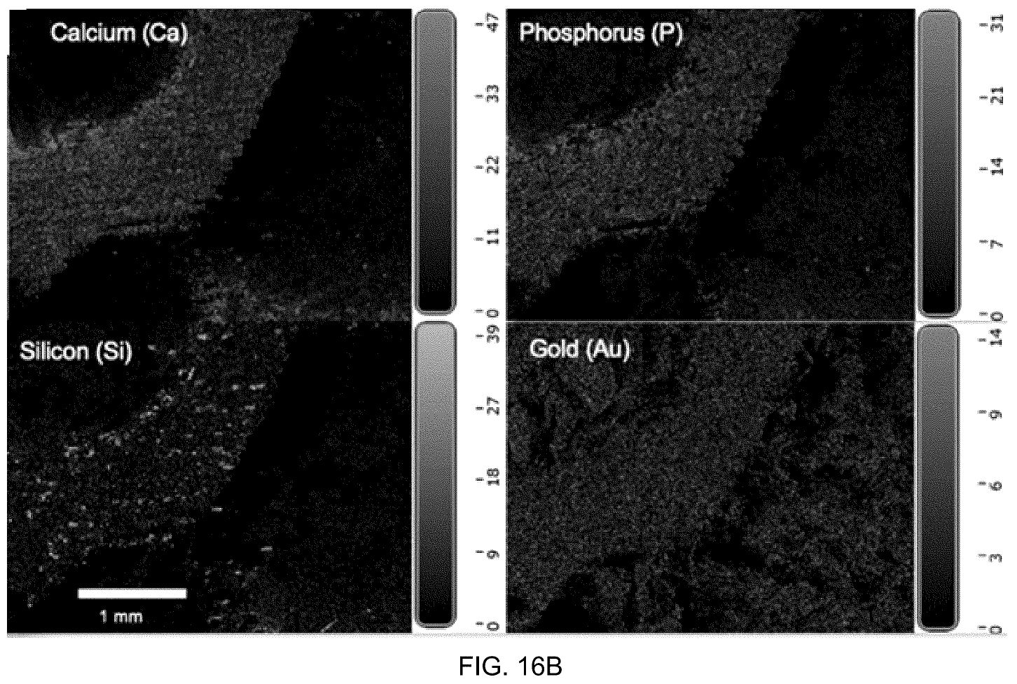

To investigate the auto-florescent characteristics of the lattice structure, its elemental composition was determined and mapped using EDS. FIGS. 16A-16B display (FIG. 16A) SEM image with EDS software mapping the visible elements; (FIG. 16B) common elements found in sample and mapped over the surface of where they are located.

The major elements found in the lattice itself were calcium (Ca), phosphorus (P) and silicon (Si) (FIG. 16B). Given that the lattice resin is hydroxyapatite, it was expected that its major elemental components would be Ca and P. The inclusion of the silicon in the resin may aid in its ability to be 3D printed (hardened) with UV light. Additionally, the silicon in the resin may be the major component that contributes to the lattice fluorescing at 405 nm.

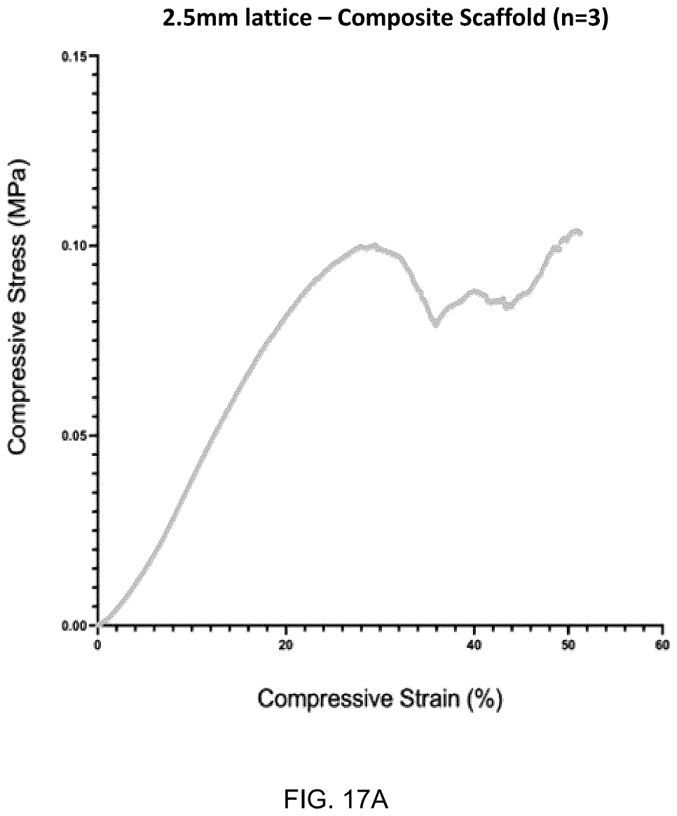

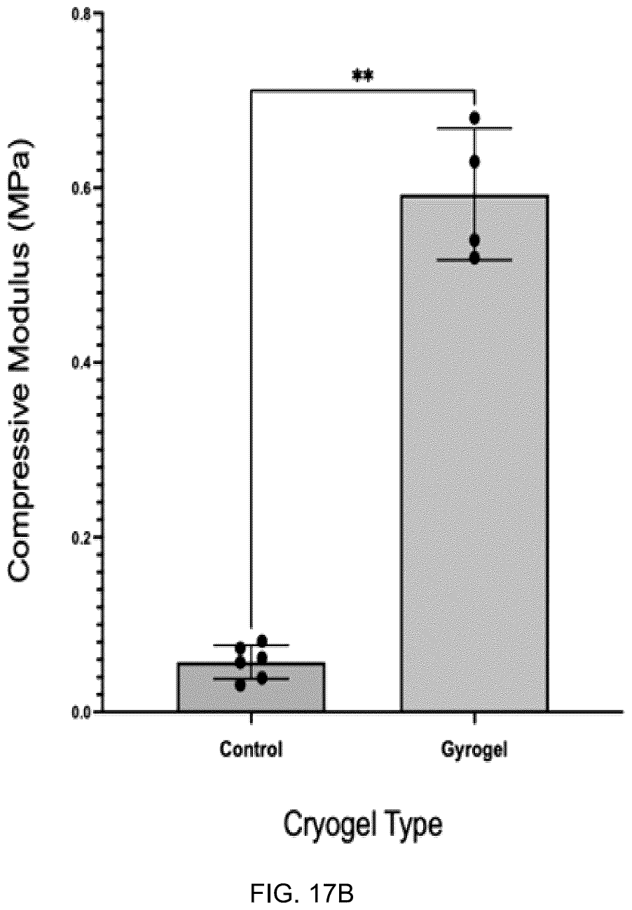

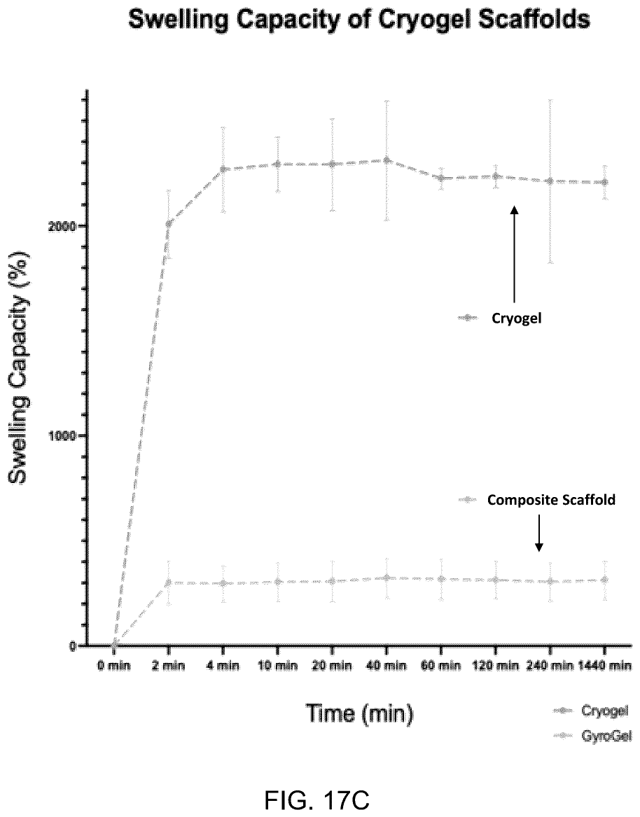

Stress, strain, and compressive modulus values of the composite bone graft scaffolds were calculated by compressing scaffolds to 50% compressive strain (FIGS. 17A-17B). The stress versus strain graph exhibited a steady increase in stress until distinct drops show when layers of the lattice structure fracture during compression. Composite bone graft scaffolds were significantly stiffer than controls, with compressive moduli values of the composite bone graft scaffolds around 0.6 MPa, which were about 100× higher than the value of controls. Finally, the swelling capacity of the composite bone graft scaffolds significantly decreased compared to controls (FIG. 17C).

Specifically, FIGS. 17A-17C display stress-strain curve of composite bone graft scaffolds (n=3). FIG. 17B shows compressive moduli values of composite bone graft scaffolds compared to control. FIG. 17C shows swelling capacity of the composite bone graft scaffolds compared to control.

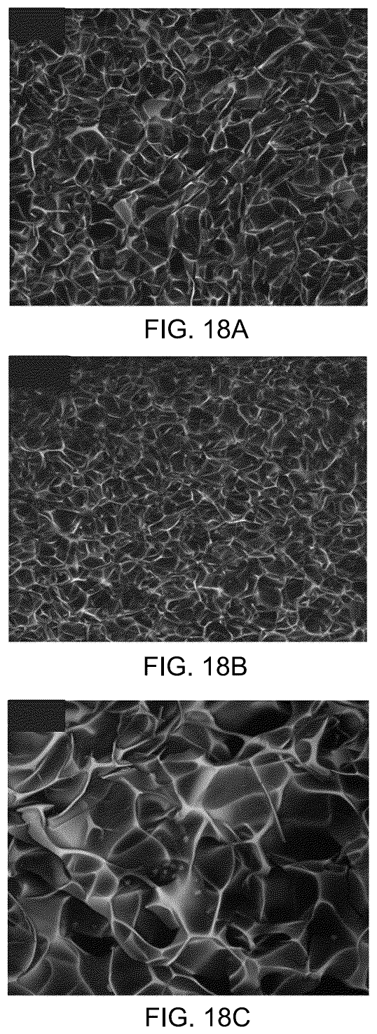

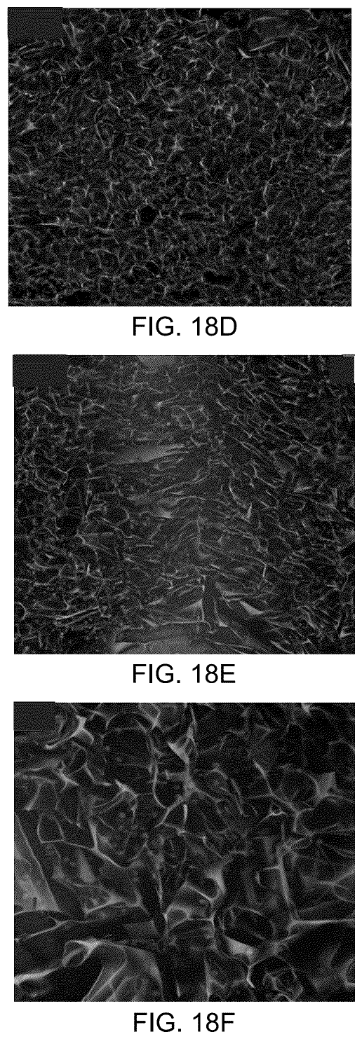

Both composite bone graft scaffolds and control scaffolds were seeded with MG63 osteosarcoma cells and incubated for 6 days. After staining cell nuclei with DAPI, and processing with TDE, scaffolds were imaged using a spinning disk confocal microscope. FIGS. 18A-18F display (FIGS. 18A-18B) control scaffolds at 10× and (FIG. 18C) 20× magnification; and (FIG. 18D-18E) composite bone graft scaffolds at 10× and (FIG. 18F) 20× magnification after 6 days of incubation.

As shown, qualitatively, the composite bone graft scaffolds have significantly more cells adhered and proliferating throughout the scaffold compared to a control. Increased adhesion in composite bone graft scaffolds may be due to the increased surface area from the lattice structure. However, FIG. 18D depicts a significant group of cells proliferating and adhered to a section of the cryogel with no lattice structure backbone.

A composite bone graft scaffold, combining cryogel scaffolds with a 3D printed mineral lattice structures can provide a mechanically stable and biocompatible environment for bone tissue regeneration. The composite bone graft scaffolds exhibited favorable physical properties (stiffness and pore size) as well as improved cellular adhesion and proliferation.

### Example 3

This example provides a description of an embodiment of a bone graft sample, such as a composite biomaterial bone graft scaffold (or, composite bone graft sample, or bone graft sample) for use in bone degeneration of segmental defects.

Reconstruction of long bones after critical bone loss continues to be a major challenge in orthopedic treatment. Specifically, a segmental bone defect is defined as a gap in bone tissue that is around two times larger than the diameter of the tissue. In general, a bone defect of this size will not heal on its own and requires surgical intervention to regain stability and function. Bone loss at this scale is most commonly the result of acute high-energy trauma, bone tumor resection, bone infection, and/or necrosis. The required limb salvaging surgical intervention involves the replacement and reconstruction of the missing bone with metallic implants (intramedullary nail (IM), open reduction and internal fixation (ORIF) or endoprosthesis) in combination with an autologous bone graft. However, the integration of bone graft material is commonly indicated for defects 6 cm in length or smaller. Therefore, segmental defect reconstruction presents an additional challenge of securing a graft in place while providing the bone necessary support and stability.

Surgeons have recently adopted a method of reconstructing segmental defects with 3D printed cages, such as lattice structures. These cages, printed from titanium or polycaprolactone (PCL) are engineered to encapsulate bone graft material of patient specific defect geometries. Cages provide a rigid structure that mimics bone shape while enabling the graft access to nutrient and waste exchange, as well as a vasculature source. While the use of 3D printed cages has shown improved outcomes by augmenting the mechanical integration of bone grafts with the metallic implants used in reconstruction, standard bone graft materials possess limitations including multiple surgeries, donor site morbidity, limited supply of tissue (major bone loss), non-union, increased risk of infection, and potential graft rejection. Thus, complications rates for bone grafts (auto and allograft), bone transport, and fibular graft are 26%, 62%, and 38% respectively. These limitations support a pressing need for the development of alternative graft materials for bone regeneration. Such approaches must aim to provide a biocompatible and mechanically stable environment for regeneration, while also promoting the differentiation/proliferation of bone cells with reduced risk of a negative immune response.

Tissue engineering is a new and rapidly growing field that combines biomaterial scaffolds, cells, and bioactive factors to repair, replace, or regenerate tissue. These properties support tissue-engineered scaffolds as an effective alternative solution to bone grafts, with the potential to promote robust cellular growth and integration at bone interfaces. In particular, cryogel scaffolds are durable, porous materials that render them as an optimal alternative to bone graft.

This example shows that the combination of cryogel with a 3D printed mineral lattice structure provides a mechanically stable structure while significantly increasing compressive stiffness. Further, this example demonstrates the potential to form cryogel integrated with a hydroxyapatite lattice structure at increasing length scales, where the integrated 3D printed lattice structure will provide the stability and support required to fill a clinically relevant femoral segmental defect.

First, this example characterized the porosity and mechanical integrity of composite bone graft scaffolds at increasing sizes (i.e., 8×10, 12×15, 16×20, 32×40, 44×60 and 44×88 mm) where the final composite bone graft scaffold is the size of a segmental defect in the femur of an average male. Embodiments of the present disclosure have shown successful fabrication of composite bone graft scaffolds at a tenth of the relevant clinical scale. This example establishes the 3D printing and cryogellation parameters required to successfully fabricate integrated composite bone graft scaffolds at increasing length scales. It is hypothesized that a 44×88 mm integrated composite bone graft scaffold can be successfully fabricated by multi-stage ceramic printing combined with custom, non-insulating 3D printed molds for full scale flash freezing during cryogellation.

Second, this example evaluated translation potential of patient specific structures by fabricating three composite bone graft scaffolds and performing accuracy of fit, volume, and integration tests within molded defect sites and relevant metallic implants using retrospective computed tomography (CT) scans and surgical plans. Following characterization of composite bone graft scaffolds at scale, it may be necessary to evaluate the accuracy of fit and clinical potential for integrating with standard-of-care prosthesis to reconstruct patient specific defects following major bone loss. In orthopedic reconstruction, it is usually critical that patient specific implants are within a very small spatial tolerance to decrease the likelihood of implant failure. It is hypothesized that all fabricated composite bone graft scaffolds will successfully fill molded replicate defect sites from humerus, tibia, and femur CT scans, with a normalized volume difference of less than 5% and a 1D scale factor greater than 90%. The embodiments of the present disclosure can be used across patient-specific bone defect reconstructions in orthopedic and plastic surgery applications (e.g., craniofacial procedures).

Critical bone defects pose a significant challenge in orthopedic reconstruction due to their size, complex geometries, and the limitations of current treatment methods. These defects, characterized by a gap in bone tissue that is approximately two times larger than the diameter of the tissue, often result from traumatic injuries, bone tumor resections, infections, or necrosis. Unlike smaller bone defects and simple fractures, segmental defects cannot heal on their own and require surgical intervention for stability and restoration of function.

The current standard of care either involves the use of metallic implants in combination with autologous bone grafts, outright replacement of the missing bone with an endoprosthesis, or amputation. Surgeons often employ intramedullary nails (IM), open reduction and internal fixation (ORIF), or endoprostheses to provide stability and support to the affected limb. These metallic implants serve as a framework for the reconstruction and act as a substitute for the missing bone segment. In addition, autologous bone grafts, typically harvested from the patient's own body, are used to fill the bone defect, and promote bone healing. The grafts provide biological support and facilitate the growth of new bone tissue. However, these methods have several limitations, including the need for multiple surgeries, donor site morbidity, limited tissue availability for major bone loss, non-union, increased risk of infection, and potential rejection of the graft. Additionally, securing a graft in place while providing the necessary support and stability poses an additional challenge in segmental defect reconstruction. Therefore, finding alternative graft materials and innovative approaches to enhance bone regeneration is important to address these limitations and improve the outcomes of orthopedic reconstruction for segmental bone defects. The present disclosure addresses these issues.

Tissue engineering is a developing interdisciplinary field that combines cells, factors for cell signaling, drug delivery, and biomaterials to regenerate, replace or repair tissue all over the body. In particular, several tissue engineered materials have been created to focus on bone regeneration, including cryogels. Cryogel scaffolds are made from polymer solutions that are cross-linked at sub-zero temperatures and thawed to produce a sponge-like, macroporous structure. This durable, porous material makes these scaffolds ideal for bone regeneration applications. Previous examples have demonstrated the ability to: i) create and refine cryogel scaffolds for clinical use; ii) enhance cryogel mineralization in vitro; and iii) use cryogels to promote bone regeneration in animal models. With their ability to promote strong cellular integration, proliferation, and growth at bone defect sites, tissue-engineered cryogels offer a new and effective alternative to autologous bone grafts.

This example demonstrates that combining cryogels with a 3D printed mineral lattice structure results in a mechanically stable structure with increased compressive stiffness that is capable of swelling. Additionally, qualitative cell studies indicate cellular adhesion and proliferation of osteoblast like cells on the surface of the composite scaffolds after 21 days.

This example investigates the is potential to form cryogels integrated with a hydroxyapatite lattice structure at increasing length scales, where the bone graft scaffold will provide the necessary stability and support to fill a clinically relevant femoral segmental defect. Successful outcomes will demonstrate the feasibility of using a composite bone graft scaffold made from tissue engineered graft material combined with a 3D printed mineral lattice structures for segmental defect reconstruction. This can be applied to patient-specific bone reconstruction in orthopedic and plastic surgery procedures, such as craniofacial procedures.

An embodiment described in this example demonstrates (1) improved porosity and biocompatibility of cryogel scaffolds to induce bone growth; (2) cryogellation with 3D printed hydroxyapatite lattice structures for increased mechanical integrity and seamless integration with existing metallic implants and surgical procedures; and (3) low cost and quick fabrication methods to achieve patient specificity.

With preliminary data indicating desirable physical properties and cellular interactions with the composite bone graft scaffolds, this example demonstrates the feasibility of fabricating these bone graft scaffolds at clinically relevant scales. It was hypothesized that chitosan/gelatin cryogel scaffolds can be successfully fabricated and combined with patient-specific 3D printed lattice structures at relevant scales to improve healing in patients with segmental defects.

The overall premise of this example is that segmental defect reconstruction requires engineered treatment options that consider material-tissue interfaces and mechanical loading while: (i) integrating with metallic implants; and (ii) promoting bone regeneration for bone defects typically larger than 6 cm. Therefore, the central hypothesis of this work is that chitosan/gelatin cryogels combined with 3D printed mineral lattice structures will improve osteogenesis at defect sites, thereby improving overall surgical outcomes.

This example combined cryogels with 3D printed mineral lattice structures to develop a cost-effective and biologically improved patient targeted treatment option for orthopedic reconstruction. To accomplish this, first, it was demonstrated that cryogel graft material can be successfully fabricated and combined with mineral lattice structures at clinically relevant scales. Composite bone graft scaffolds were fabricated at increasing sizes and evaluated for porosity, swelling kinetics, and mechanical integrity to demonstrate successful fabrication of a desirable scaffold at clinical scale. Then patient specificity and surgical integration capability were demonstrated by testing three full scale composite bone graft scaffolds with accuracy of fit, volume, and integration tests within molded defect sites modeled after patients.

Initially, the gyroid lattice structure was selected for its interconnected porous structure and surface area-volume ratio (FIG. 5A). Initial work investigated the feasibility of fabricating chitosan/gelatin cryogel material with PLA (polylactic acid) gyroid lattice structures by allowing cryogel solution to permeate varying 3D printed gyroid structures (FIG. 5B) and freezing them together. This initial work demonstrated successful fabrication of the composite bone graft scaffolds, with SEM imaging indicating strong material integration and pore sizes around 80 μm. Additionally, mechanical testing showed increased mechanical durability with compressive modulus values around 600 times greater than cryogel alone.

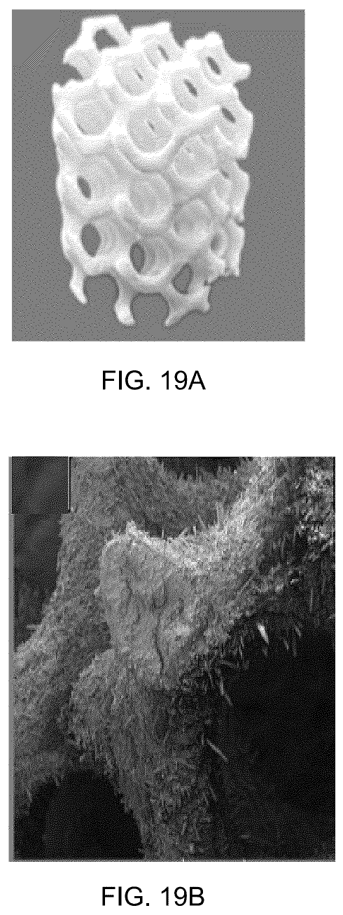

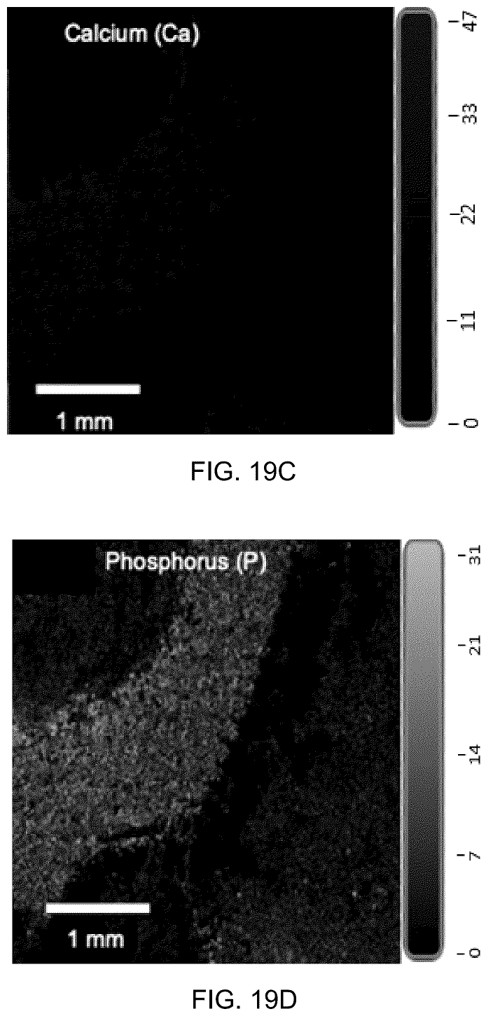

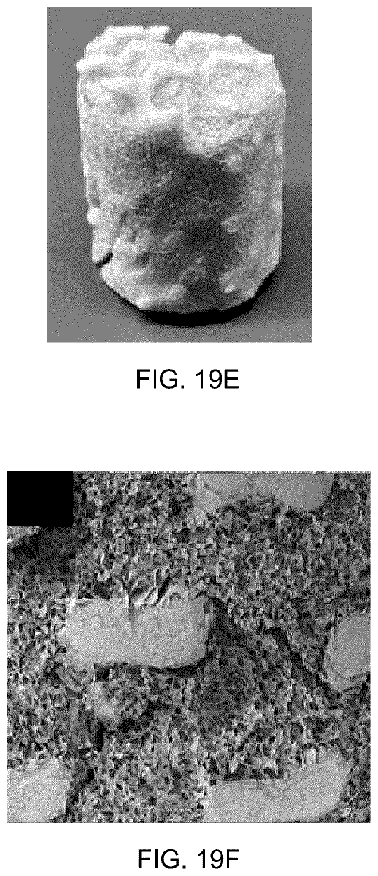

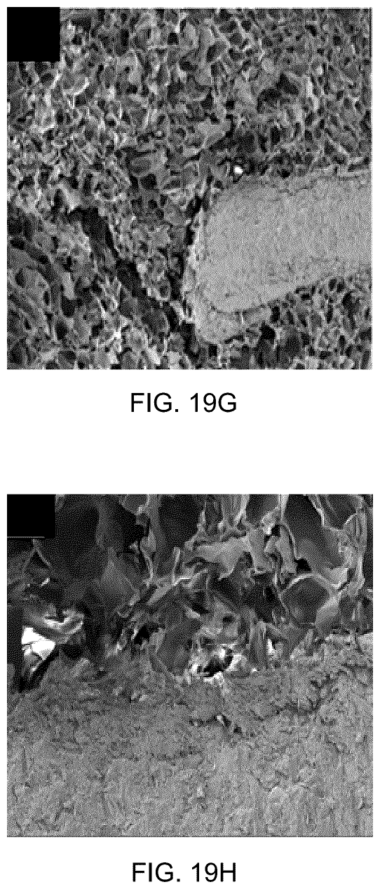

A 3D printed hydroxyapatite lattice structure combined with cryogel can decrease the potential cytotoxic effect seen from plastic while promoting mineralization of osteoblast cells. Initial examples demonstrated both the fabrication techniques and resultant physical properties of 3D printed hydroxyapatite gyroid lattices integrated with chitosan-gelatin cryogel. SEM images depict crystalline structures that make up the 3D printed lattice structures (FIGS. 19A-19B). Energy-dispersive X-ray spectroscopy (EDS) imaging confirmed the elemental makeup of the lattice structure to be mostly calcium and phosphorus (FIGS. 19C-19D). SEM images of the composite bone graft scaffolds (FIGS. 19E-19H) were quantified using a python pore analysis program (FIG. 19G) that measured the average pore diameter of the composite bone graft scaffolds at 74.56 μm with a standard deviation of 106 μm, whereas a control cryogel scaffold has an average pore diameter of 71.24 μm with a standard deviation 31.69.

Although the pore sizes for the composite bone graft scaffolds exhibited greater variance as compared to a control, both scaffold types exhibited a range of pores that fall well within the pore size range of around 100-200 μm. Stress, strain, and compressive modulus values of the composite bone graft scaffolds were calculated by compressing scaffolds to 50% compressive strain (FIGS. 17A-17B). Stress-strain results indicated a lower yield strength as compared to plastic lattice structures but remained elastic at higher strains. Mineral composite bone graft scaffolds were significantly stiffer than controls, with compressive moduli values around 0.6 MPa, which were about 100× higher than the value of controls. Like composite bone graft scaffolds using plastic lattice structure cryogels, while absolute swelling is decreased compared to a control, composite bone graft scaffolds using mineral lattice structure cryogels achieve absolute swell capacity, 300% of their dry weight, in 2 minutes (FIG. 17C).

Composite bone graft scaffolds and control scaffolds were seeded with MG63 osteosarcoma cells and incubated for six days. Confocal fluorescent images of the surface of the composite bone graft scaffolds samples indicated cell adhesion and proliferation after six days (FIGS. 18D-18F). Overall, the incorporation of a 3D printed mineral lattice structures within a cryogel scaffold does not appear to adversely affect pore formation. This demonstrated that fabricating tissue-engineered composite bone graft scaffolds made of 3D printed hydroxyapatite gyroid lattice structures and chitosan-gelatin cryogel can be used as a bone graft material for segmental bone defect reconstruction.

The porosity, swelling capacity and mechanical integrity of composite bone graft scaffolds were characterized at increasing sizes (i.e., 12×15, 16×20, 32×40, 44×60 and 44×88 mm) where the final composite bone graft scaffolds are the size of a segmental defect in the femur of an average male. It was hypothesized that a 44×88 mm integrated composite bone graft scaffold could be successfully fabricated by multi-stage ceramic printing combined with custom 3D printed molds for full scale flash freezing during combined cryogellation.

Initial examples have shown successful fabrication of composite bone graft scaffolds at a tenth of the relevant clinical scale. Next, 3D printing and cryogellation parameters to successfully fabricate integrated composite bone graft scaffolds at increasing length scales were determined. Cryogel scaffold material was created through the crosslinking of a polymer solution, where immediate freezing/subsequent thawing results in a sponge-like, mechanically durable, macroporous structure (FIG. 3). These properties of cryogels are critical for achieving a graft material well-suited for bone regeneration.

To effectively achieve cryogellation of a composite bone graft scaffold with a volume equal to 133 cm³, adequate fabrication parameters were established. Composite scaffolds composed of 3D printed hydroxyapatite lattice structures permeated with chitosan/gelatin solution were frozen in cylindrical geometries at increasing sizes (i.e., 1.7, 4, 32, 91, and 133 cm³). The composite bone graft scaffolds were imaged by SEM and microCT to quantify porosity (N=3/group). Cyclic loading and ultimate compression were used to assess mechanical integrity (N=6/group). Following loading, the composite bone graft scaffolds constructs were placed in sterile phosphate buffered saline (PBS) to quantify the degradation potential over time (N=3/group).

In vitro characterization of composite bone graft scaffolds at increasing sizes may ensure that as the overall volume of the graft increases, cryogel formation, swelling capability, pore distribution and mechanical integrity will not be compromised for inducing bone formation. It is believed that the increasing scale will not have a detrimental effect on cryogel formation and its physical properties.

Translation potential of patient-specific structures was evaluated by fabricating three composite bone graft scaffolds and performing accuracy of fit, volume, and integration tests within molded defect sites and relevant metallic implants using retrospective computed tomography (CT) scans and surgical plans. It was determined that all fabricated composite bone graft scaffolds will successfully fill molded replicate defect sites from humerus, tibia, and femur CT scans, with a normalized volume difference of less than 5% and a 1D scale factor greater than 90%.

Following characterization of composite bone graft scaffolds at scale, the accuracy of fit and clinical potential for integrating with standard-of-care prosthesis to reconstruct patient specific defects following major bone loss were evaluated. In orthopedic reconstruction, it is usually critical that patient specific implants are within a small spatial tolerance to decrease the likelihood of implant failure. Utilizing retrospective surgical plans and CT scans of patient specific defect sites (N=3), full scale negative molds of the defect site post debridement were 3D printed. With patient data, segmented 3D CT scans, and surgical information on the metallic implants and graft geometries used to reconstruct the site, modeled gyroid lattice structures were 3D printed from hydroxyapatite and filled with cryogel solution to replicate the geometry. Fabricated composite bone graft scaffolds were tested to fit into the molded defect sites, in combination with the relevant metallic implants utilized in the surgery to simulate full scale reconstruction. The simulated surgical reconstruction was analyzed for accuracy of fit, volume difference, and scale factor.

If scaling of the composite bone graft scaffolds adversely affects the homogenous pore distribution of the cryogel due to uneven freezing, composite bone graft scaffolds molds or the lattice structures may need be re-designed to allow for more even distribution of heat transfer. One method can use a two-stage freeze cycle where the center of the composite bone graft scaffolds is left hollow, and a heat transfer material (e.g., ethanol) can be directed through both the center of the mold and around the entire structure to enable flash freeze of the entire structure. The core of the composite bone graft scaffolds can then be frozen separately, and inserted into the graft after cryogellation is complete.

### Example 4

This example provides a description of the fabrication and characterization of embodiments of the present disclosure, such as 3D printable ceramic lattice structures for bone tissue engineering applications.

Three-dimensional (3D) printed lattice structures for bone tissue engineering (BTE) applications have attracted attention for their ability to provide a patient-specific construct to aid in bone regeneration. Lattice structure fabrication has mainly focused on the use of hydroxyapatite (HA) as the main component of lattice structures due to the compound's similarity to the inorganic component of natural bone. Despite its popular use however, HA possesses some non-ideal properties, including its slow in vivo degradation rate and high price. This example provides a description of embodiments of 3D-printable mineral lattice structures using alternative mineral sources to hydroxyapatite, namely, wollastonite (CaSiO₃), and β-TCP (Ca₃(PO₄)), in order to increase accessibility while demonstrating a bone-like gyroid lattice structure for mechanical support. Fabricated lattice structures were printed on a digital light processing (DLP) printer to achieve solid gyroid lattice structures. Pieces were then subjected to a sintering cycle to enhance mechanical properties and eliminate the polymer component of pieces, resulting in lattices composed solely of mineral. Overall, β-TCP lattice structures exhibited greater mechanical strength after sintering compared to all other lattice structure compositions. The shrinkage post sintering, as noted in macroscopic observations, may explain this increase in mechanical strength. All scaffolds exhibited cellular adherence and proliferation at days 4 and 7, demonstrating that all lattice structures exhibited osteoconductive properties within this early time frame. This example also discusses embodiments for a method for fabricating cost-effective mineral lattice structures utilizing DLP 3D printing technology for BTE applications.

### Bone Physiology and Injury

Healthy bones are essential for providing support to the human body, where typical composition includes a hard, outer shell (i.e., cortex), with a spongy, mesh-like inner structure (i.e., cancellous/trabecular). Due to their mechanical strength, bones are often mischaracterized as static structures. However, bones function dynamically to maintain mineral homeostasis, serve as a site for hematopoiesis, and undergo remodeling in response to physiological demands. Specifically, bones are continuously undergoing remodeling at their surface, replacing old bone with new bone through the coordinated activity of osteoblasts and osteoclasts. Further, physical activity and mechanical demands during growth and aging can direct bone modeling to shift directions of growth as well as bone mass distributions. Compositionally, bones consist of both inorganic and organic portions which are approximately 60 and 30% by weight, respectively. The organic component of bone is composed of over 30 proteins, the main component being type 1 collagen with an abundance of over 90%. The primary component of the inorganic portion is calcium phosphate in the form of crystalline calcium phosphate, also known as hydroxyapatite.

Healthy bone readily heals through a tightly organized, systematic process. However, when bones are injured beyond the capacity of this healing response, the quality of life of a patient can be severely compromised. These large bone defects, also known as critical-sized defects, can result from trauma, infection, tumors, or congenital diseases, among other factors. A critical-sized defect is typically regarded as one that does not heal solely with surgical stabilization, requiring additional treatment with bone graft materials to facilitate regeneration and healing. Annually, four million people across the globe require bone transplantation or replacement surgery.

Despite their common use, auto-, allo-, and xeno-grafts each present their own unique challenges. Allografts, materials harvested from the same species, are often expensive and come with a risk of immune response and rejection; whereas xenografts, materials harvested from a different species, carry the risk of transmitting zoonotic diseases, and have an even greater risk for host rejection. The current gold standard technique for treating critical-sized bone defects is autologous bone grafting, which involves harvesting bone from a site in the body to be used at the injured site of the same patient. Though this technique is considered the gold standard, the extra surgical site presents risks for pain, donor-site morbidity, vessel injuries, and infection. For these reasons, biosynthetic alternatives are being investigated to replace the need for autologous bone grafting. Moreover, the field of bone tissue engineering (BTE) has been investigating the role of three-dimensional (3D) printing to allow for personalized bone graft scaffolds based on anatomical imaging.

3D printing is a technology that allows for the material production of complex structures with high precision. Its application in BTE involves the combined usage of medical imaging techniques, such as computed tomography (CT) scan or magnetic resonance imaging (MRI), along with computer-aided design (CAD) software to create patient-specific models. Currently the major 3D printing techniques for polymer scaffold construction include fused-deposition modeling (FDM), selective laser sintering (SLS), and digital light processing (DLP). FDM is the most popular technique and involves an extruder tip that melts plastic which is then deposited and solidified on a cooler surface. Note that this technique is limited in resolution and by the number of biocompatible, medical-grade thermoplastic polymers available for printing via this method. Comparatively, SLS uses a laser to sinter powder layer by layer, forming the final shape. However, this then requires cleaning to remove excess powder and achieve a smooth surface. Lastly, DLP printing constructs 3D models by using a digital projector screen to cure a cross-sectional layer of a photosensitive base, building up the desired object layer by layer. This technique confers several advantages, including improved surface resolution, precision, and speed. Because of these advantages, DLP was the chosen technique to be employed in this example.

Overall, DLP printing is considered a type of vat polymerization, in which objects are constructed from a vat of photopolymerizable resin base. A typical resin used in this technology consists of oligomers, monomers, initiator, and, in the case of a ceramic resin, a mineral. When the resin is irradiated with ultraviolet (UV) light, the initiator releases a free radical which reacts with monomers to initiate a polymerization reaction. Next, chain formation occurs, and monomers and oligomers are converted to polymers, rendering a liquid to solid phase change. The polymerized component will then form a network that will encase the mineral particles in a network, forming a solid structure. For the application of 3D printing in BTE, work has more recently focused on incorporating synthetic calcium phosphate minerals within the resin as they mimic the native inorganic component of bone (hydroxyapatite). Therefore, hydroxyapatite (HA) additive is most widely used due to its chemical similarity to the inorganic component of bone [Ca₁₀(PO₄)₆(OH)₂]. Note that unlike synthetic HA, bone mineral is compositionally nonstoichiometric due to the dynamic exchange of cations and anions within the crystal lattice. The 3D printed lattice structures containing HA demonstrate high porosity and pore interconnectivity, and the addition of HA has been shown to promote revascularization, osteoconduction, and osteointegration. Despite these advantages and wide use in bone tissue repair, there are several drawbacks to the properties of HA, including its high thermodynamic stability. As these ceramic scaffolds are intended to provide temporary support during the healing process, scaffold should be designed to degrade as host cells deposit extracellular matrix (ECM) on its surface, eventually being replaced by natural bone. Overall, HA does not easily break down under physiological conditions and is highly brittle, making it non-ideal for bone regeneration applications. Further HA is more expensive than other calcium phosphate sources, reducing accessibility. Therefore, this example investigates alternative bioceramic materials for BTE applications: beta-tricalcium phosphate and wollastonite.

Both beta-tricalcium phosphate and wollastonite provide cost-effective alternatives to HA for use in BTE. Beta-tricalcium phosphate (β-TCP) is a mineral with the formula Ca₃(PO₄)₂. It is highly biocompatible and osteoconductive and can be used to produce synthetic bone graft scaffolds. Compared directly to HA, β-TCP has higher biodegradability and pore interconnectivity, important components for osteoconduction, but possesses decreased mechanical properties. Therefore, this example utilized β-TCP as a calcium phosphate source in the production of BTE scaffolds. As an alternative, wollastonite (CaSiO₃) is a widely used mineral in the production of paints, coatings, dental roots, and many other industrial applications. Recently, it has been studied for its potential use in BTE applications due to its biocompatibility, biodegradability, and mechanical properties. Unlike HA, wollastonite also contains silicon, which has been shown to be important in promoting bone mineralization. Additionally, wollastonite is cost-effective and improves biodegradability when combined with HA lattice structures. This example also utilized wollastonite as a mineral in the fabrication of BTE lattice structures. Note that in order to create biocompatible bone graft scaffolds using lattice structures, there is a need to burn off the photosensitive polymer and improve mechanical properties of the lattice structures. Sintering improves the mechanical strength of 3D printed ceramic pieces of the lattice structures. Therefore, all 3D printed mineral lattice structures were sintered to provide appropriate material composition for biocompatibility.

In this example, the chosen lattice structure to be printed is a gyroid shape. The gyroid surface has been studied extensively for its properties in various disciplines where it is structurally composed of repeating triply periodic minimal surfaces. Recently, it has become the object of investigation in the field of BTE due to its potential role in promoting cell attachment, proliferation, and angiogenesis, due to its high surface area-to-volume ratio. Therefore, in this example, gyroid-shaped ceramic lattice structures were 3D printed and its properties were investigated.

In this example, three different resins of lattice structures were fabricated using β-TCP, wollastonite, and 1:1 mixture of both components together. Using these resins, gyroid lattice structures were 3D printed and sintered. These lattice structures were then evaluated for their physical properties compared to natural bone and a commercially available ceramic resin (Osteolite®). It was hypothesized that β-TCP/wollastonite 3D printed matrix lattice structures will possess comparable physical properties to Osteolite® resin, providing a cost-effective alternative. Scanning electron microscopy (SEM) was used to visualize and compare the surface of printed lattice structures, and the chemical composition of the lattice structures were also analyzed. Mechanical testing and a cell adhesion study were also carried out to evaluate the mechanical properties and biocompatibility of the lattice structures, respectively.

Three different suspensions were formulated varying the type of ceramic powder incorporated (Table 3). The wollastonite used was from Natural Pigments (Willits, CA). Because of the macro particle size, the wollastonite was ground using a mortar and pestle and sifted through a 400-mesh sieve to ensure particle sizes at or below 37 μm. In addition to this, 325 mesh B-TCP was used (Pure Bulk; Roseburg, OR) to ensure a particle size of 44 μm or less. Sodium dodecyl sulfate (SDS) was used as a dispersing agent to help ceramic particles remain suspended in the resin (Sigma Aldrich; St. Louis, MO). The photopolymerizable base used was Tethon's Genesis Development Resin Base (Tethon; Omaha, NE), which allows for the incorporation of solid powders. A formula of 45 wt % of solid powder and 0.5 wt % of dispersant were used.

Formulation began with the incorporation of powder components, where the given ceramic powder was first incorporated with SDS and shaken on an analog vortex mixer. Following complete mixing, the powder component was incorporated incrementally into the resin base with continuous stirring using a magnetic stir bar and hotplate until fully combined.

TABLE 3
Composition of fabricated resins with the 3D printing solution.

| | B-TCP content | Wollastonite content | SDS content |
|---|---|---|---|
| Name | (wt %) | (wt %) | (wt %) |
| Wollastonite | 45 | 0 | 0.5 |
| Wollastonite/β-TCP | 22.5 | 22.5 | 0.5 |
| β-TCP | 0 | 45 | 0.5 |

The 3D printed lattice structures were printed using a Bison 1000 DLP Printer (Tethon 3D; Omaha, NE). Printing parameters consisted of exposure time of 38 sec for Osteolite®, 45 wt % BTCP, and 45 wt % wollastonite resins. Exposure time was 22 sec for the 45 wt % (1:1) B-TCP and wollastonite combination resin. Following printing, pieces were detached from the build platform using a smooth-edged spatula. Supports were removed by hand, and pieces were cleaned using 99.9% isopropyl alcohol in an ultrasonic cleaner (Branson 200; Branson Ultrasonics, Brookfield, CT) to remove excess of non-polymerized suspension. After drying, pieces were subjected to a 405 nm ultraviolet (UV) lamp for 10 min to fully harden the photosensitive resin of the lattice structure.

A Fourier-transform infrared spectroscopy (FTIR) spectrum of the printed lattice structure was obtained (FT/IR-6200; Jasco, Oklahoma City, OK) with an ATR accessory to analyze the chemical composition of printed pieces, both pre- and post-sintering. Printed samples were crushed into a fine powder using a mortar and pestle before being placed onto the ATR accessory for analysis.

TG analysis was performed on lattice structures before sintering (TGA 550; TA Instruments, New Castle, DE).

The surface of printed lattice structures pre- and post-sintering were visualized using scanning electron microscopy (SEM VEGA3 TESCAN; Brno, Czech Republic). Samples were mounted on an aluminum stub and sputter coated for 240 sec in gold at 15 mA (HUMMER 6.2; Anatech, Sparks, NV). Images were then obtained using SEM at 15, 200, 1000, and 2000× magnification.

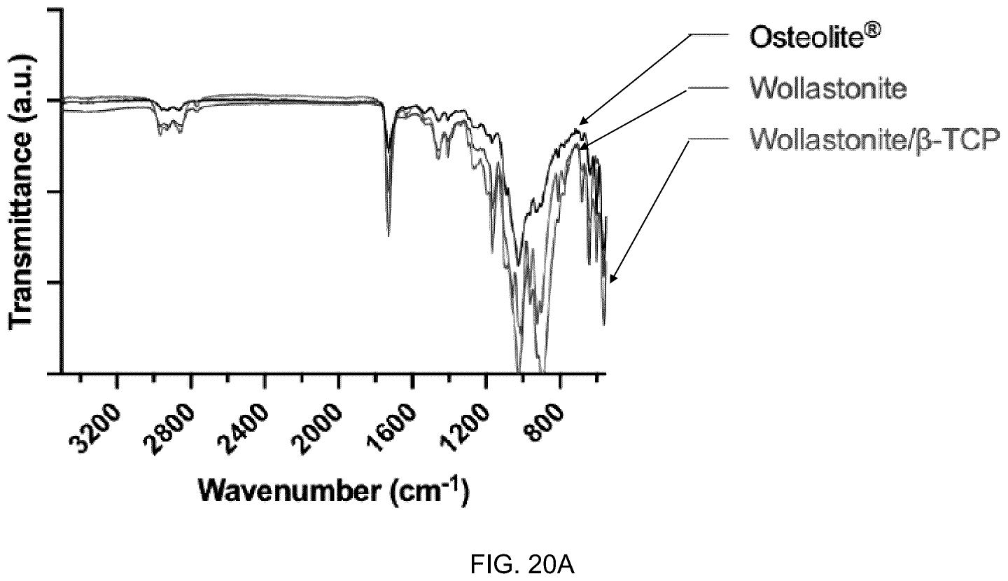

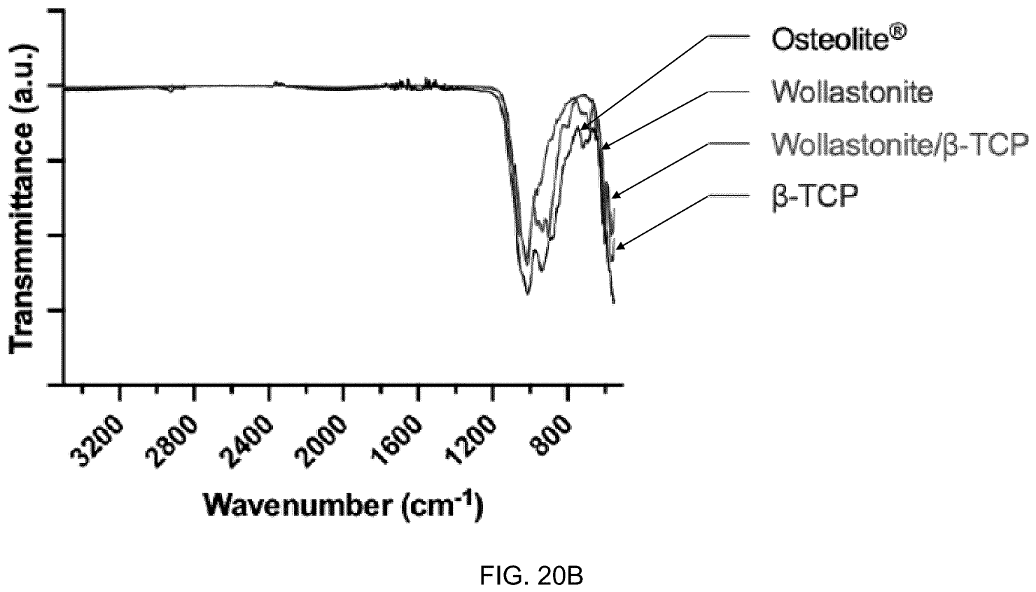

Pieces were sintered to i) remove the organic polymer component and ii) improve the mechanical properties of the gyroid lattices. The organic components of printed pieces were first removed by a heating scheme indicated in FIGS. 20A-20B. Following this, the lattice was subject to a sintering cycle consisting of a ramp rate of 10° C./min, holding at 1100° C. for 5 hours, then a cooling rate of 10° C./min.

FIG. 20A displays FTIR curves of printed lattice structures before sintering, and FIG. 20B displays FTIR curves of printed lattice structures after sintering.

Compression was completed using an Intron 68SC-2 system (Instron; Norwood, MA) with a 500 N load cell and a test rate of 10 mm/min, preload of 0.05 N, and preload speed of 1 mm/min. Data was analyzed through the Bluehill universal software (Instron; Norwood, MA), and the compressive modulus (MPa) was taken from the software output.

A gyroid lattice structure design, as described herein, was used. The lattice structures printed were cylinders with a 10 mm height and 9 mm diameter.

A total of 12 samples (N=3 per resin group) were sterilized in 70% ethanol (Fisher Scientific; Fair Lawn, NJ, USA) for 30 min, followed by three, ten-min washes with sterile phosphate buffered saline (PBS). The lattice structures were then placed in a sterile 24-well plate (Falcon; Marlboro, NY, USA) with 100 L of Dulbecco's Modified Eagle's Medium (DMEM; ThermoFischer Scientific, Waltham, MA, USA), 10% fetal bovine serum (Omega Scientific Inc.; Tarzana, CA, USA), and 1% penicillin-streptomycin solution (Life Technologies Corporation, Carlsbad, CA, USA). Each lattice structure was seeded with 50,000 human bone osteosarcoma-derived cells (MG-63, passage 97; ATCC; Manassas, VA, USA) through a dropwise method and left to incubate for two hours at 37° C. and 5% CO₂ to ensure cellular attachment. Then, an additional 200 L of complete media was added so that all samples were fully submerged. Media changes were conducted every 2-3 days. The lattice structures and media were cultured for 4 and 7 days, at which time the lattice structures were placed in formalin (Fisher Scientific; Fair Lawn, NJ, USA) for 24 hours for fixation, and then stored in PBS. Cellularized lattice structures were imaged using a confocal microscope (Andor W1 Spinning Disc Confocal Microscope; Oxford Instruments, Abingdam, United Kingdom).

GraphPad Prism was utilized to conduct all statistical analyses, with a significance level of 0.05. A two-way ANOVA, followed by a Tukey post hoc analysis, was performed to assess the significance among groups for compression testing analysis.

To analyze the chemical identity of the formulated resin, Fourier-transform infrared spectroscopy (FTIR) analysis was performed on printed lattice structure, both pre- and post-sintering (FIGS. 20A-20B). FTIR of unsintered lattice structures show peaks characteristic of a carbon polymer chain (FIG. 20A), with peaks corresponding with sp3 and sp2 C—H bonds, at 2967 cm⁻¹ and 2864 cm⁻¹, respectively. A peak corresponding with a carbonyl group is visualized at 1730 cm⁻¹, as well as a peak at 1169 cm⁻¹ corresponding with skeletal chain vibration (FIG. 20A).

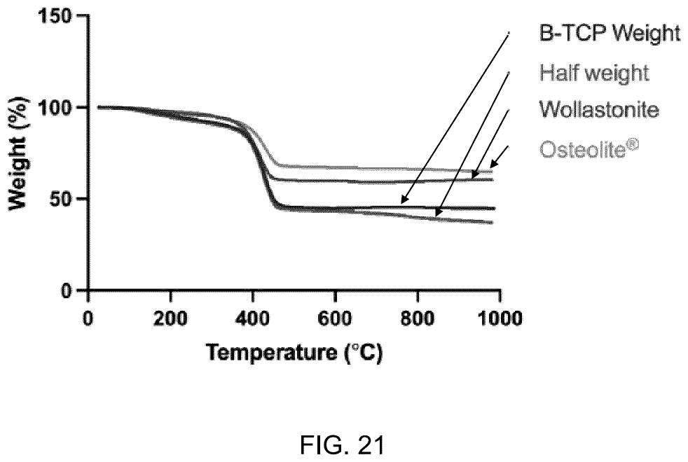

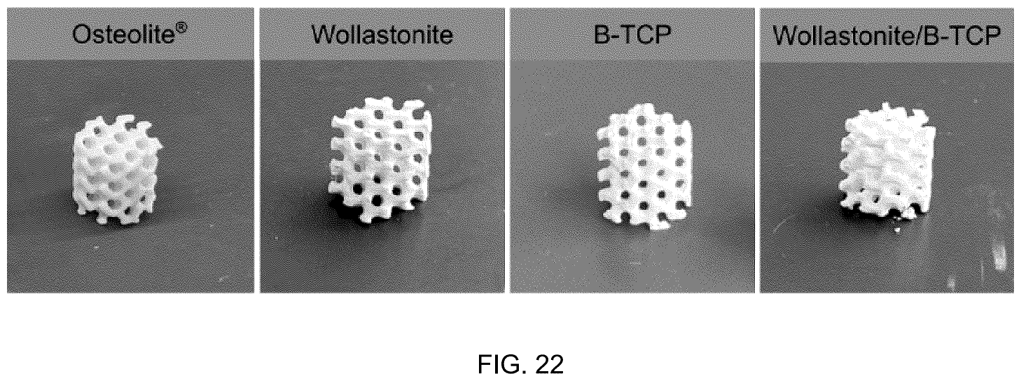

TG analysis of printed lattice structures before sintering confirmed the onset temperature for the combustion of the organic component of the resin. This was indicated by weight loss beginning at 390° C. for all lattices (FIG. 21).

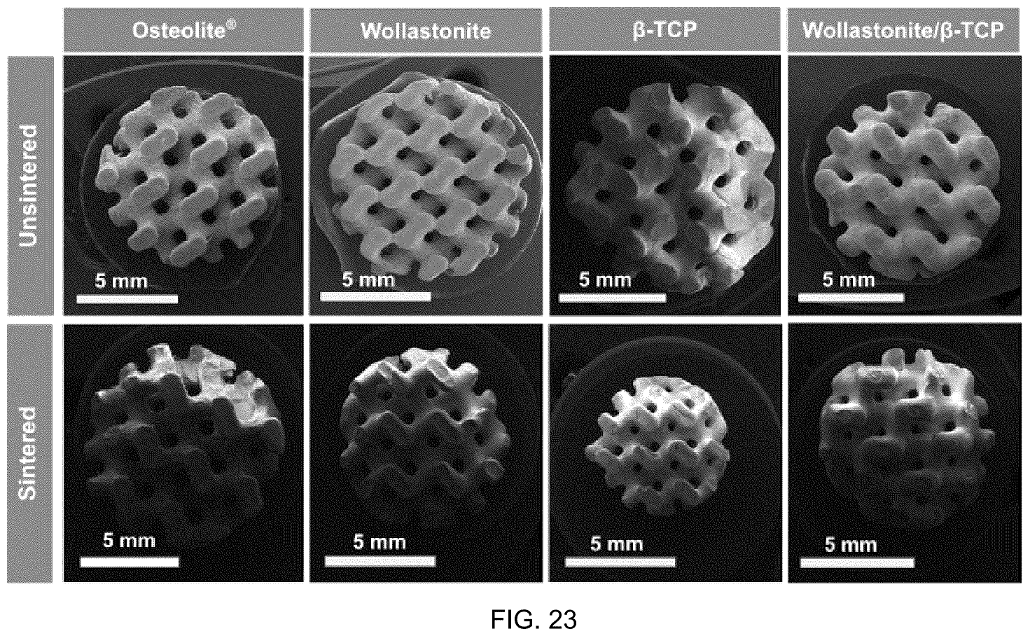

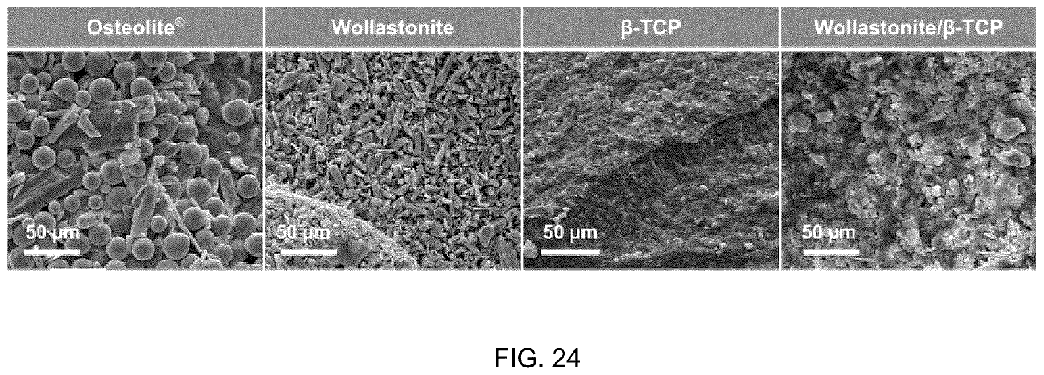

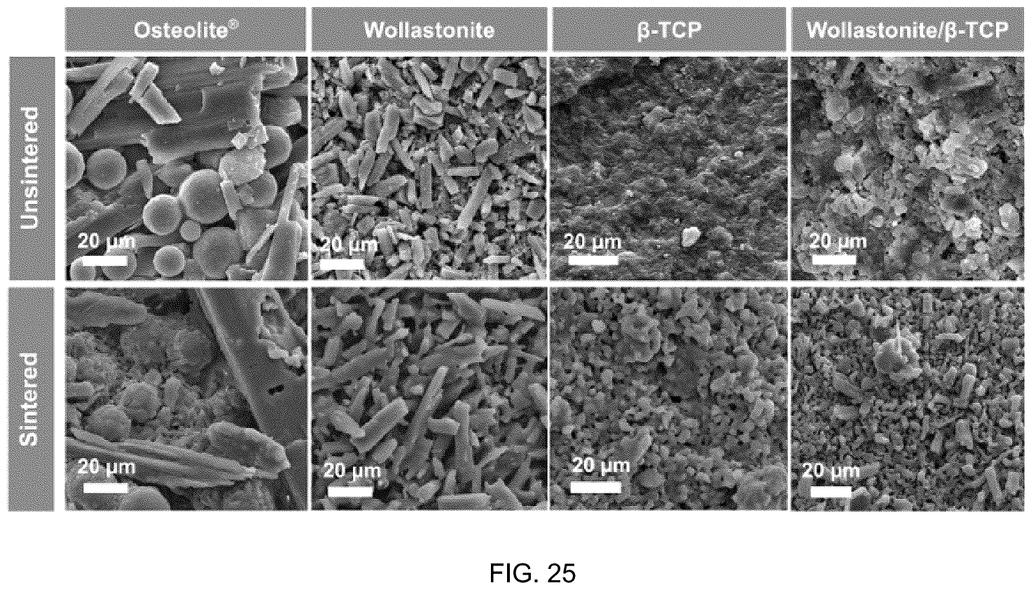

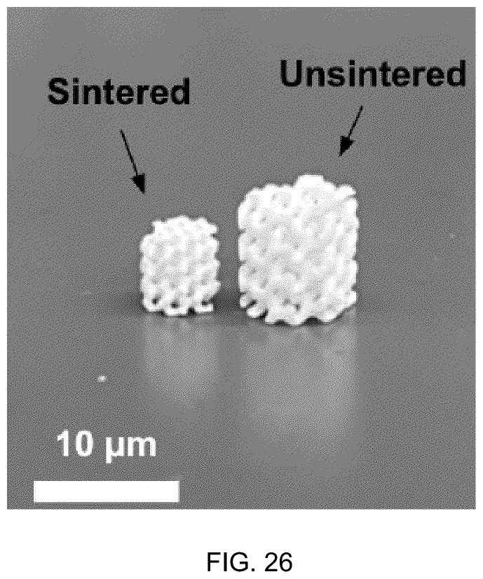

Macroscopic pictures of the printed lattice structures are depicted in FIG. 21. The overall physical appearance between groups is not dramatic. However, color differences are noted macroscopically (FIG. 22). SEM images depicting lattice structures at 15× show the different surface morphologies of the printed lattice structures (FIG. 23). Comparatively, SEM images taken at 1000× and 2000× allow for closer examination of surface morphologies of the different lattice structures. Osteolite® appears to contain hydroxyapatite particles between 10 and 20 μm, and particles of wollastonite at 100 μm and larger (FIGS. 24 and 25). For the gyroid lattice structures printed using all wollastonite resin, the average particle size is between 20 m and 40 μm. As for the lattice structures printed using solely β-TCP resin, the particles are indistinguishable from each other before sintering due to the presence of the carbon polymer encasing the particles (FIGS. 24 and 25). After sintering, however, pores emerge due to the removal of the organic polymer. SEM images of the wollastonite/β-TCP combination resin depicted what looked like β-TCP with larger, crystal-like wollastonite particles scattered throughout. A changed surface morphology is also noted between unsintered and sintered lattice structures (FIGS. 24 and 25). It must be noted that gyroid lattice structures printed with β-TCP resin exhibited marked shrinkage after sintering, which could be visualized macroscopically (FIG. 26).

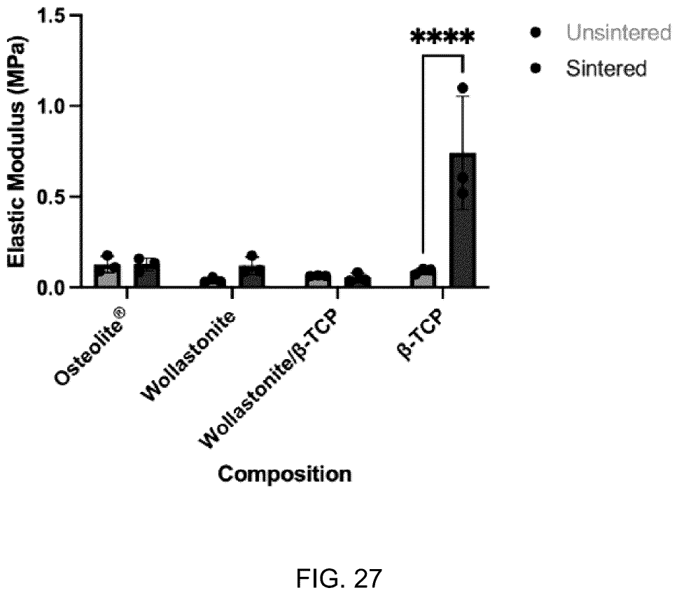

Mechanical properties of lattice structures were evaluated before and after sintering. The compressive modulus for β-TCP lattice structures increased after sintering (p<0.0001; FIG. 25). In unsintered lattice structures, Osteolite® possessed a higher modulus than the Wollastonite lattice (p<0.05), but no significance was found in comparison to other lattice structures (FIG. 26). Post-sintering, the β-TCP gyroid lattice structures exhibited a higher compressive modulus compared to all other lattice structures (p<0.01) (FIG. 27).

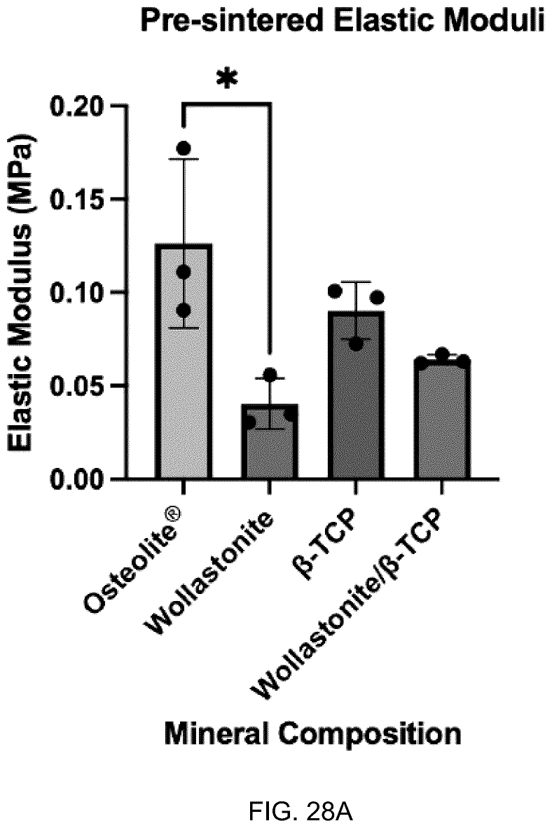

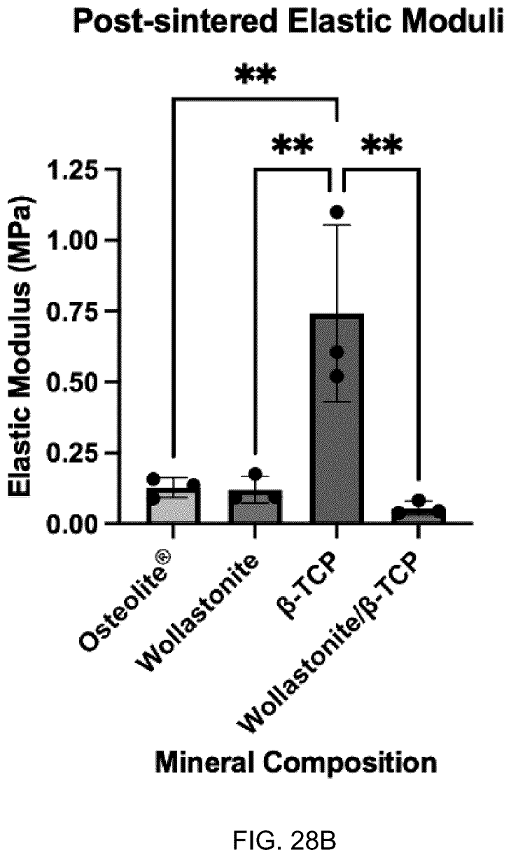

All lattice structures were seeded with MG-63 osteosarcoma cells and cultured for 7 days. Confocal images depict cellular adherence to all lattice structures at day 4 (FIG. 28A). At 7 days of incubation, lattice structures showed a qualitatively denser population of cells on the surface of the lattice structures, demonstrating cellular proliferation at this time point.

In this example, three resins were fabricated by incorporating mineral components (Wollastonite, β-TCP, and a 1:1 combination of both minerals) into a polymerizable base to achieve a 3D printable ceramic resin. Resins were printed using a DLP printer to achieve solid, 3D gyroid lattice structures. These lattice structures were then subject to a sintering scheme, which removed the polymer component, resulting in fully mineral-composed gyroid lattice structures. The mineral lattice structures were evaluated and compared to lattices of the same shape printed with Osteolite® resin to evaluate mechanical properties and biocompatibility. Overall, HA is a widely studied mineral in BTE for its similarity to the inorganic component of native bone. However, it does not easily break down under physiological conditions and is highly brittle, making it nonideal for bone regeneration applications. This example investigated two alternative minerals to HA, wollastonite, and β-TCP. Wollastonite (CaSiO₃), a calcium silicate, was chosen for its mineral properties. Notably, wollastonite provides a source of silicon that more conventional minerals, such as HA and β-TCP, lack. Silica can be used in promoting cell adhesion, proliferation, and angiogenesis. Further, silicon has been found to be an initiator of mineralization. β-TCP (Ca₃(PO₄)₂) was studied due to its desirable properties, such as being highly bioresorbable. β-TCP has higher biodegradability than hydroxyapatite, making it a good candidate in fabricating BTE lattice structures and/or bone graft scaffolds, which ideally degrade as new bone is formed. Further, these materials are far more cost-effective than HA, rendering them promising candidates for fabricating cost-effective BTE bone graft scaffolds using lattice structures.

Chemical characterization of the fabricated lattice structures first began with FTIR analysis of printed pieces prior to sintering. These results showed peaks that confirmed the presence of a polymer chain and, by cross-checking these vibrational modes with literature, allows for confirmation of the presence of poly(methyl methacrylate), also known as PMMA. PMMA is a thermoplastic polymer often used in long-term orthopedic applications for its biocompatibility and long-term stability in the body. For BTE applications where lattice structures are intended to serve as temporary matrices, it is advantageous to eliminate this highly stable polymer component to allow for degradation to occur. Further, results of TG analysis confirmed the onset temperature for the combustion of the organic component to be at 390° C. (FIG. 21). The chosen sintering cycle for this example exceeds this temperature, supporting that full elimination of PMMA from the lattice structures was achieved. FTIR analysis further verifies this with the disappearance of peaks (as shown in FIG. 20A) corresponding to PMMA in lattices after sintering, confirming the elimination of the polymer from the sintered lattice structures (FIG. 20B).

Following chemical analysis, surface morphology is observed post-sintering (FIGS. 24 and 25) in all lattice structures. Further, SEM images taken at 2000× demonstrate micropores in the post-sintered β-TCP lattice structure with diameters of approximately 5 μm (FIG. 25). Previous literature indicates that a range of pore sizes confer different advantages for bone graft scaffolds having lattice structures in bone regeneration. Pores larger than 300 μm are recommended for allowing vascularization and bone cell infiltration, but micropores have also been shown to possess desirable properties. Specifically, studies have shown that micropores less than 10 μm increase surface area and can promote bone protein absorption and ion exchange.

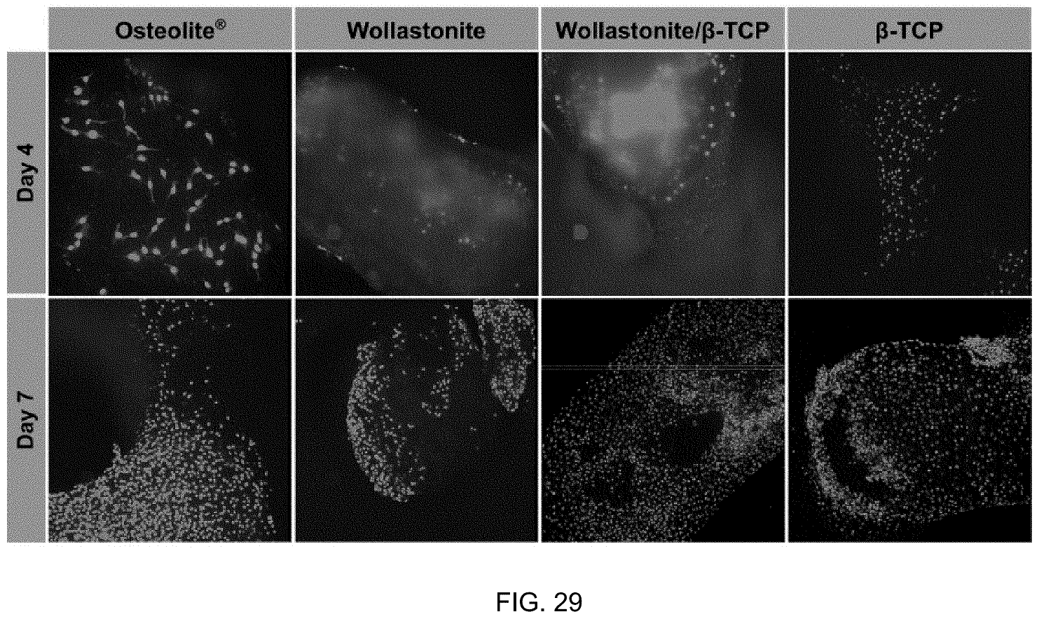

In addition to surface morphology and chemical composition, an important consideration for fabrication of BTE bone graft scaffolds using lattice structures is the mechanical strength of the fabricated pieces of lattice structures. Ideally, the mechanical strength of lattice structures will mimic that of natural bone. When examining the mechanical properties of lattice structures before sintering, Osteolite® had the highest modulus and was stronger than the Wollastonite lattice (p<0.05; FIGS. 26 and 29). Following sintering, the mechanical strength of the β-TCP lattice structure was improved (p<0.0001). However, this trend was not reflected with the other lattice structures (FIG. 27). Similarly, when comparing the compressive modulus across lattice structures of different compositions post-sintering, the β-TCP lattice structure had a higher compressive modulus compared to all other lattice structure (p<0.01) (FIGS. 28A-28B). Further, shrinkage of the β-TCP lattice structure post-sintering was observed macroscopically (FIG. 26). Shrinkage in ceramic lattice structure, and specifically those of β-TCP composition, have been noted in literature. Shrinkage of ceramic pieces during sintering likely indicates an increase in density that may result in the observed increase in mechanical strength post-sintering. The sintering temperature can vary between materials, and the β-TCP may have reached its sintering temperature during the sintering cycle applied in this study, while the other lattice structures did not. Sintering wollastonite structures at 1250° C. may confer the most mechanical strength, but other temperatures can be used.

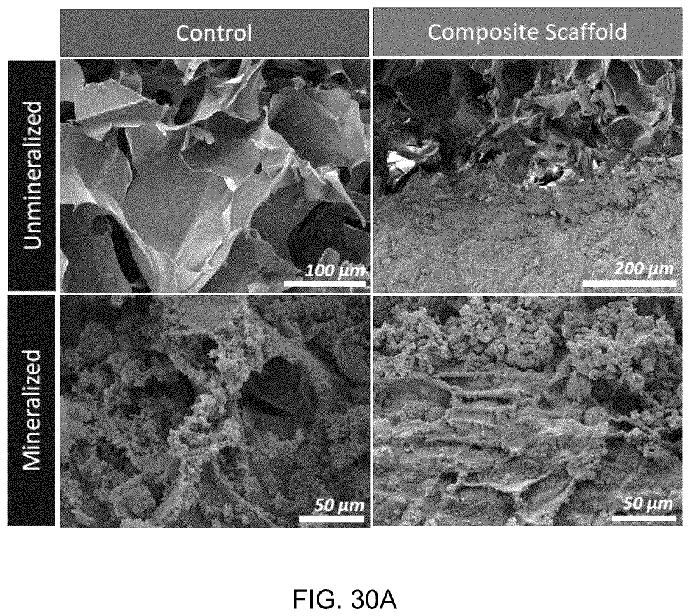

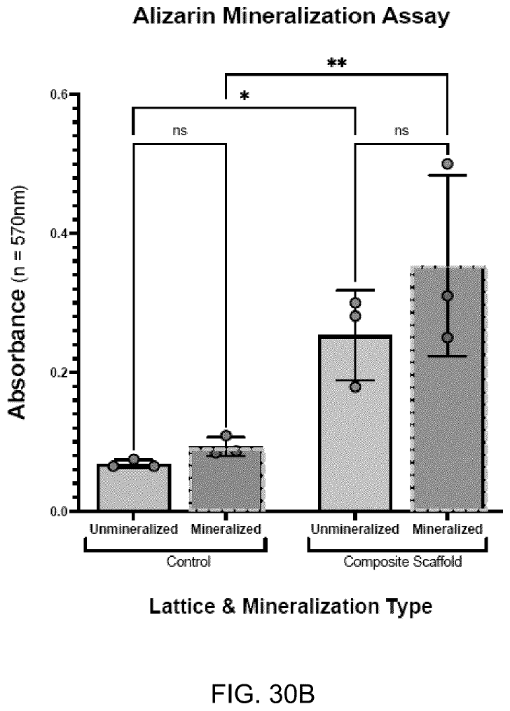

For bone graft scaffolds including lattice structures to successfully promote bone regeneration and healing, bone cells must be able to tolerate and adhere to surface of bone graft scaffolds. A biocompatibility study using MG-63 osteosarcoma cells was performed on lattice structures of each different resin composition. In the first four days, MG-63 (bone-like) cells exhibited adherence across all four scaffolds including the lattice structures, with cells distributed across the scaffold surfaces and penetrating towards the center of the scaffolds (FIGS. 30A-30B). Notably, of the different compositions of lattice structures, Osteolite® and β-TCP lattice structures exhibited the highest observable cell density, with the wollastonite/β-TCP combination lattice structures exhibiting an intermediate number of cells. While the wollastonite lattice structure showed cell adherence across its surface, cell distribution was sparser compared to the other four lattice structure types. At day 7, cells had continued to adhere and proliferate on the surface of the scaffold having the lattice structures, exhibiting a qualitatively increased surface density of cells on all scaffolds having the lattice structures. The relative amounts of qualitatively observable numbers of cells followed the same trend as in day 4 cells across groups, but all groups experienced an increase in cell density (FIGS. 30A-30B). These results taken together allow for the conclusion that fabricated scaffolds having lattice structures demonstrate osteoconduction at days 4 and 7. Further, the shrinkage seen in β-TCP did not negatively impact cell response to these scaffolds having lattice structures, supporting its use in bone formation.

It is desirable to develop an alternative to autologous bone graft use in the treatment of large bone defects. Though several commercially available synthetic bone grafts exist, their high price and reduced accessibility necessitates a better alternative. This example aimed to fabricate and characterize 3D-printable mineral resins using alternative mineral sources to hydroxyapatite, namely, wollastonite and β-TCP, to increase accessibility while demonstrating a bone-like gyroid lattice structure for mechanical support. Fabricated resins were printed on a DLP printer to achieve solid gyroid lattice structures. These pieces were then subjected to a sintering cycle to enhance mechanical properties and eliminate the polymer component of pieces, resulting in lattices composed of solely mineral. Overall, β-TCP lattice structures exhibited greater mechanical strength after sintering compared to all other lattice compositions. It is likely that the shrinkage post sintering, as noted in macroscopic observations, explains this dramatic increase in mechanical strength. As for biocompatibility, all scaffolds having the lattice structures exhibited cellular adherence and proliferation at days 4 and 7, demonstrating that all lattice structures exhibited osteoconductive properties within this early time frame. Overall, this example demonstrates a promising method for fabricating cost-effective mineral bone graft scaffolds utilizing DLP 3D printing technology for BTE applications.

### Example 5

This example provides a description of the mechanical strength and short-term and long-term interactions and viability of a composite bone graft scaffold as described herein. In this example, the composite bone graft scaffold is a 3D printed hydroxyapatite (HA) lattice structure with chitosan-gelatin (CG) cryogel, as described herein.

As shown in FIGS. 30A-30B, bone graft scaffolds exhibit increases in mechanical durability and mineralization, as compared to the control. Specifically, FIG. 30A displays the control and composite bone graft scaffolds SEMs when unmineralized and the crystal polymorph creation following soaking in Ca²⁺ and PO₄³⁻ solutions. FIG. 30B displays the absorbance values of ARS bound to calcium salts.

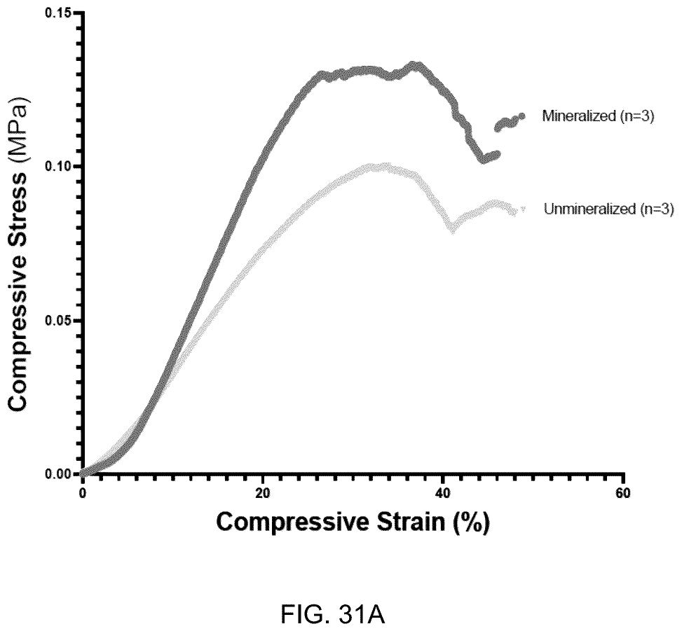

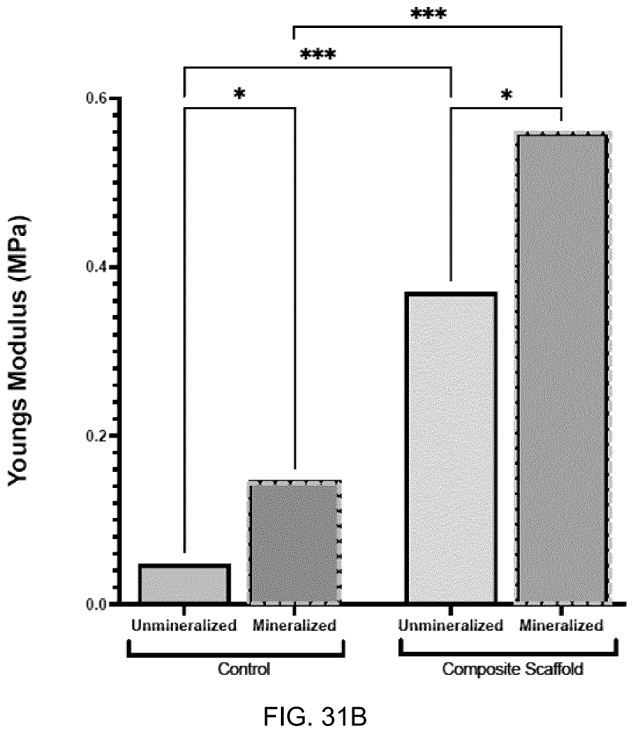

Further, FIG. 31A displays compressive stress-strain curves of composite bone graft scaffolds. FIG. 31B displays the Young's modulus values following compression tests for composite bone graft scaffolds and control scaffolds. As shown, the composite bone graft scaffold is stiffer than the control, highly resilient at larger strains compared to a 3D printed mineral lattice alone, and stiffer than itself following mineralization dunking.

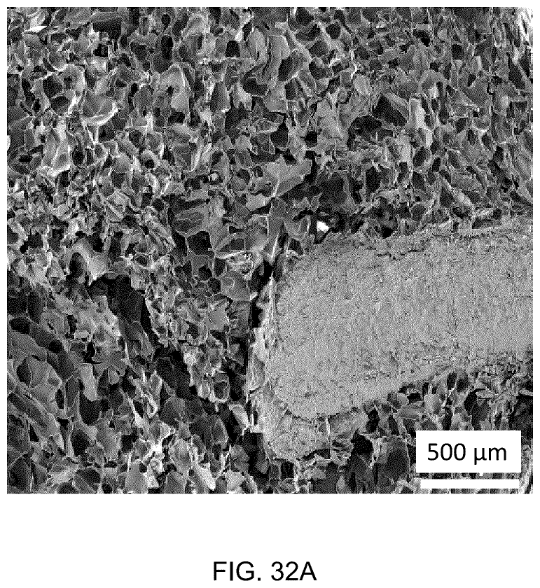

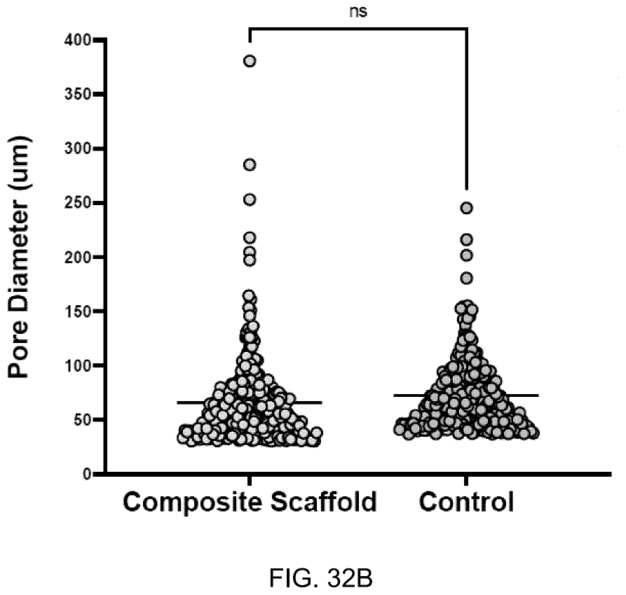

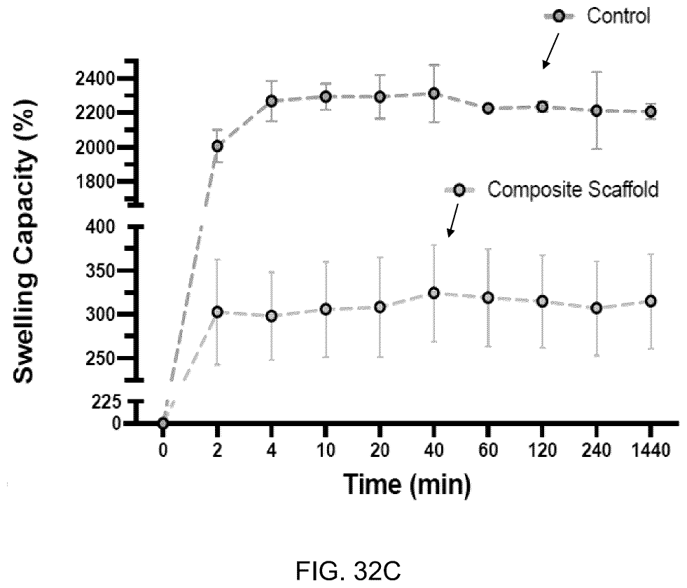

It was found that the composite bone graft scaffolds exhibit non-significant differences in average pore diameter. FIG. 32A displays a SEM of pore composite bone graft scaffold measurements. FIG. 32B displays the pore diameter values. FIG. 32C displays the swelling kinetics.

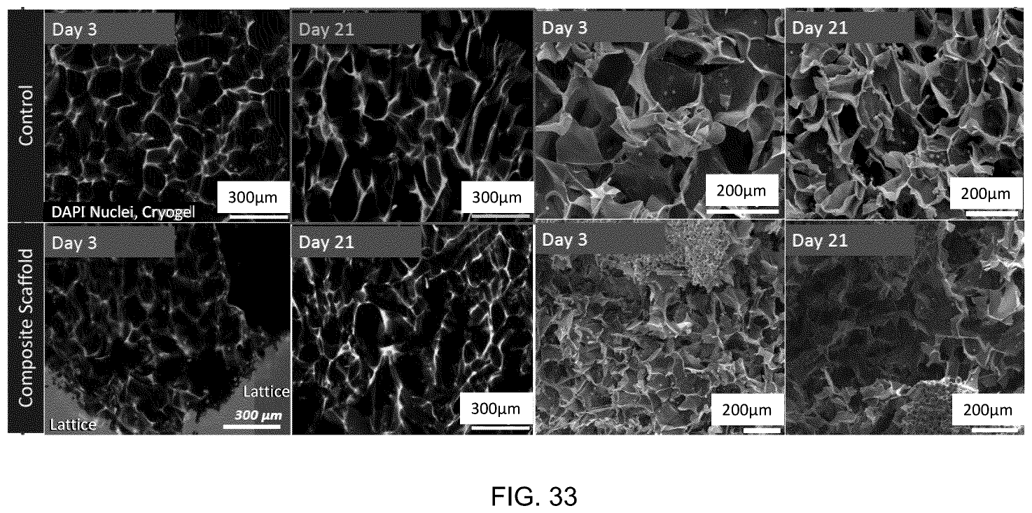

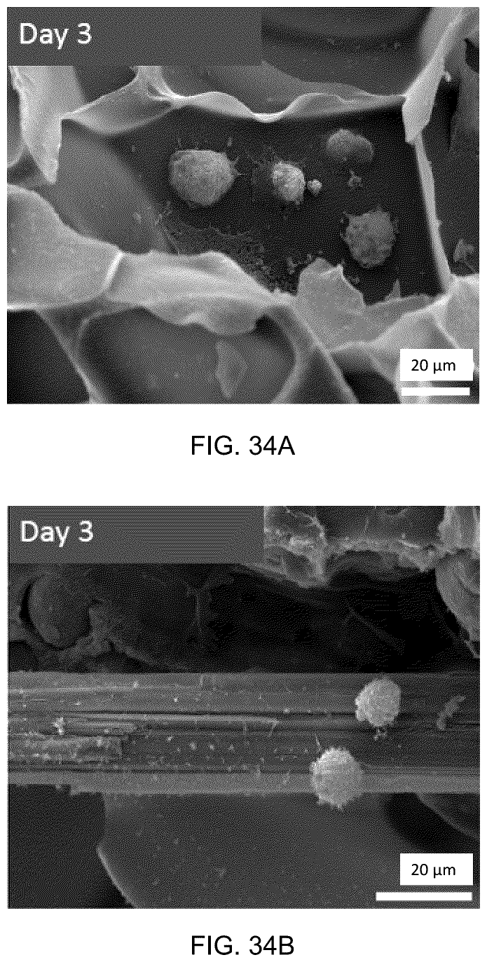

Further, scaffolds were seeded with MG-63 osteoblast-like cells and incubated for β-, 6-, 7-, 14- and 21-day timepoints, as shown in FIG. 33. As shown in FIGS. 34A-34B, there was strong cellular adhesion to both cryogel and mineral lattice structure surfaces. Specifically, FIG. 34A displays a SEM image of MG-63 cells adhered to the cryogel and FIG. 34B displays a SEM image of MG-63 cells adhered to the 3D printed HA lattice structure.

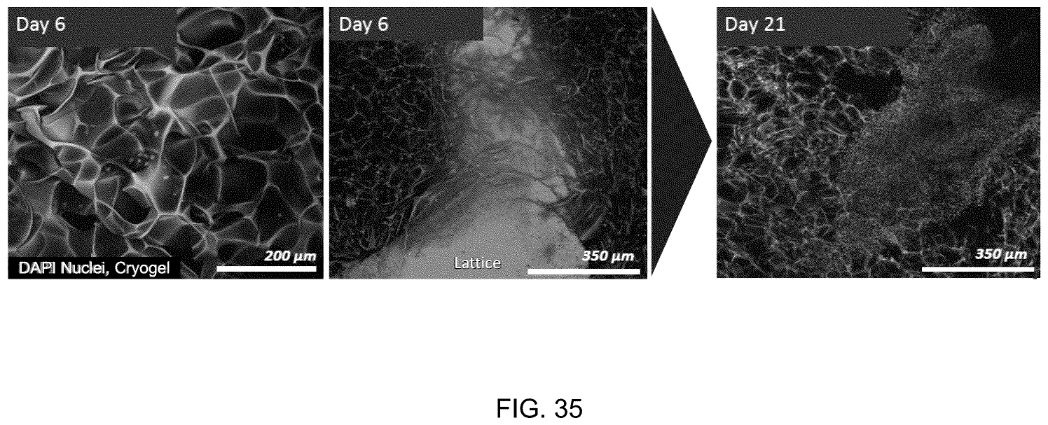

SEM images show cell adhesion and viability up to 21 days on the cryogel and the 3D printed mineral lattice structure. As shown in FIG. 35, there was limited proliferation for cells after day 6 for the control and composite bone graft scaffolds. The control and composite bone graft scaffolds exhibited little cellular proliferation over time as compared to scaffolds including cryogels.

### Example 6

This example provides a description of mineralized and non-mineralized composite bone graft scaffolds, as described herein.

Bone grafting is a critical procedure employed to address various congenital, traumatic, and pathological craniofacial defects. However, current limitations of traditional bone grafting techniques, such as donor site morbidity, inadequate tissue integration, and limited graft availability, necessitate the exploration of innovative alternatives to meet the growing clinical demand. Cryogel scaffolds are formed through a process of freezing and thawing a cross-linked polymer solution, leading to a highly porous and sponge-like structure that facilitates cellular infiltration and angiogenesis. Moreover, the integration of natural materials, such as chitosan and gelatin, enhances the scaffold's biocompatibility, biodegradability, and non-toxicity.

The bone graft scaffolds disclosed herein may be customized to replicate bone defect sites using patient-specific molds. The current conventional method for mandible reconstruction involves a bone graft accompanied by a surrounding titanium mesh, with an optional bone morphogenetic proteins (BMP) slurry. This example discusses a composite bone graft scaffold that emulates this standard practice using bioactive materials: hydroxyapatite (HAp), calcium metasilicate (CaSiO₃), and BMP-2. Patient-specific cryogels with mineral-inducing additives within 3D printed lattice structures can improve osteogenic potential, stimulate bone regeneration, and enhance graft integration within the host tissue.

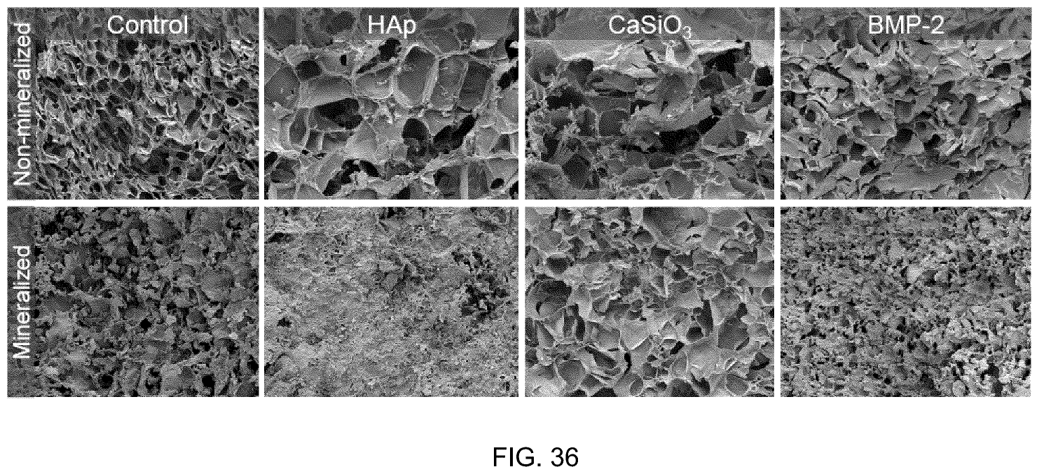

As shown in FIG. 36, additives (hydroxyapatite (HAp), calcium metasilicate (CaSiO₃), and BMP-2) changed the morphology and mineralization potential of cryogels.

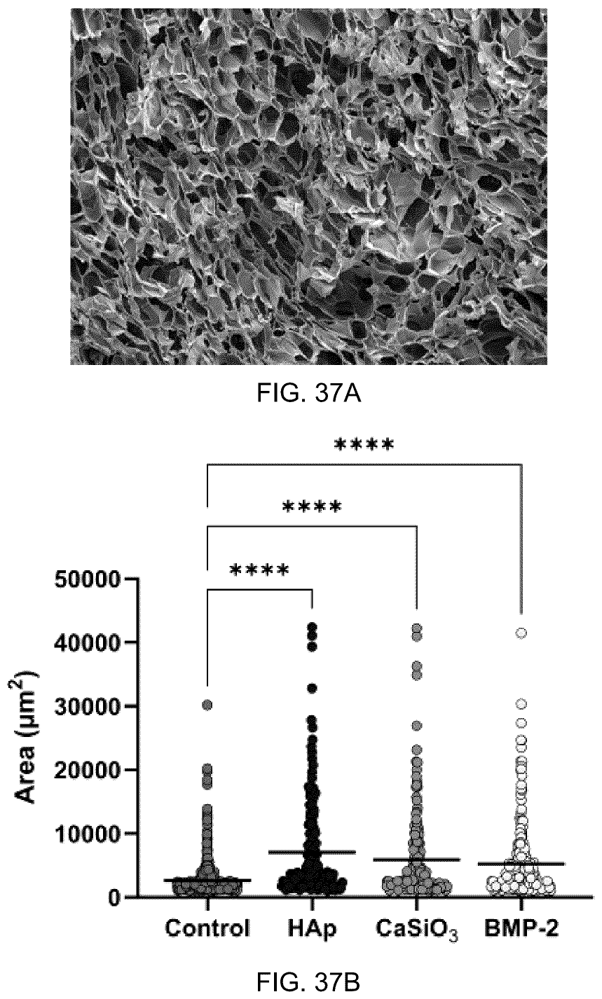

As shown in FIGS. 37A-37B, additives increased the composite bone graft scaffold pore area. Specifically, FIG. 37A displays a python-generated pore analysis program output of a cryogel and FIG. 37B displays a pore area distribution of cryogels with additives (solid line is the mean value (p<0.05)).

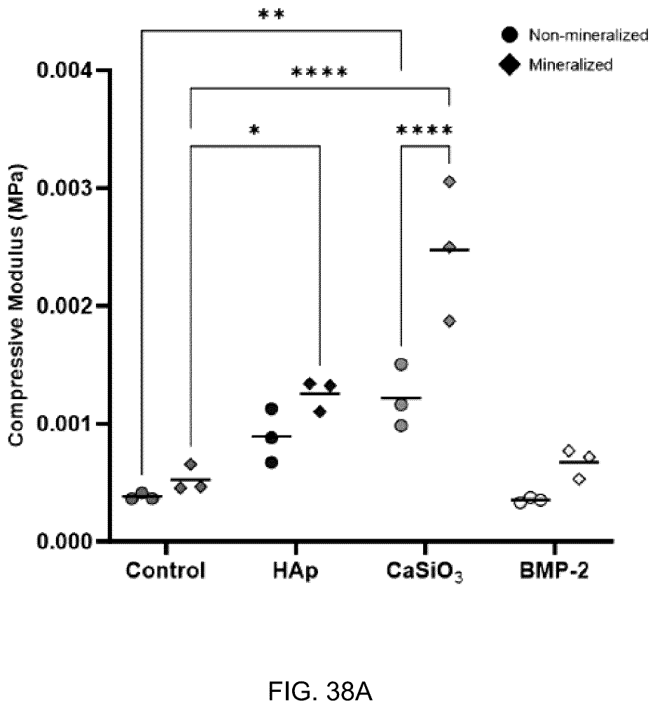

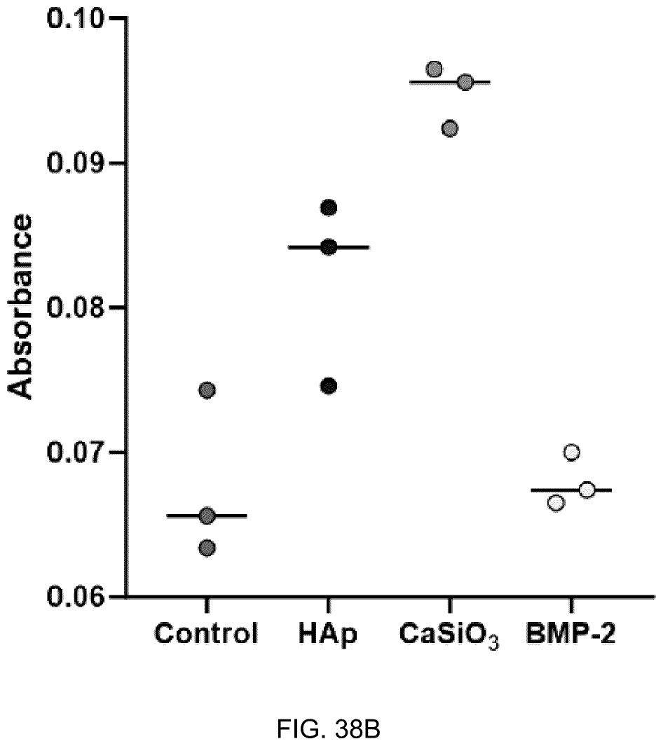

As shown in FIGS. 38A-38B, HAp and CaSiO₃ increased the mechanical strength of cryogels. Specifically, FIG. 38A displays a compressive modulus of cryogels with additives calculated from a stress-strain curve generated by an Instron 68SC-2 system (p<0.05). FIG. 38B displays the absorbance of cryogels with additives calculated using ARS staining and SpectraMax Microplate Reader.

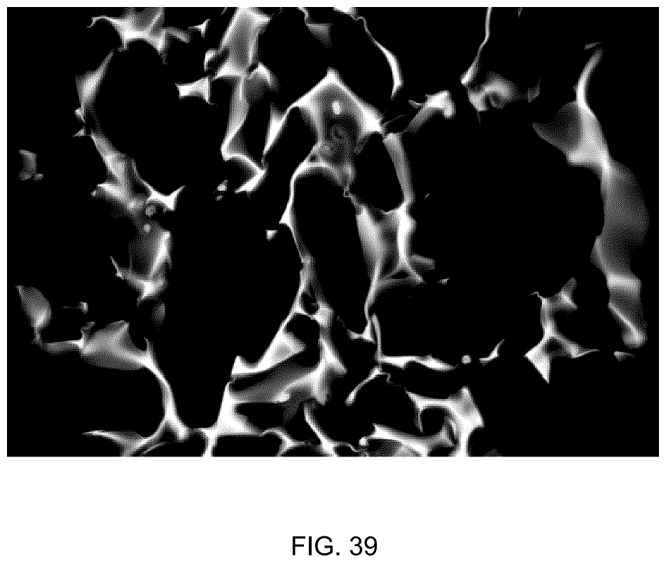

FIG. 39 displays cell infiltration-stained images using DAPI staining and light microscopy. Individual fluorescent blue dots indicate the nuclei of the MG-63 cells. The green-yellow structures indicate the composite bone graft scaffold. From this, it was found that cells can infiltrate and proliferate within gyroid structures.

These results provide evidence for reliable formation of cryogels with mineral-inducing additives as well as customizable 3D printed scaffold structures. It was found that pore size increases with all three additives, compressive modulus increases with the addition of HAp and CaSiO₃, mineral content increases with the addition of HAp and CaSiO₃, and cells can infiltrate and proliferate within combined cryogel and customized 3D printed bone graft scaffolds.

### Example 7

This example provides a description of sintered and unsintered lattice structures, as disclosed herein.

Embodiments of the present disclosure include a 3D-printed mineral lattice structure framework composed of 4:1 hydroxyapatite-wollastonite, designed for Digital Light Processing (DLP) 3D-printing, containing a binder consisting of carbon-based methacrylate oligomers and a photoinitiator diphenyl(2,4,6-trimethyl benzoyl)phosphine oxide for curing a resin.

It has been found that materials in the binder may affect the 3D-printed lattice structure's density and strength and are cytotoxic. The process of sintering hydroxyapatite (Ca₅(PO₄)₃OH) has been demonstrated to rid the 3D-printed material of such binders, preserving pure hydroxyapatite with improved mechanical properties and crystalline structure. By using a combustion-sintering cycle for mineral resins, a sintered 3D-printed hydroxyapatite-wollastonite mineral lattice structures may have increased strength, clear fusion of mineral components, a reduction of the carbon-based elements from the binder, and a reduction of peak intensity for hydroxyapatite and wollastonite crystals, as compared to an unsintered lattice structure.

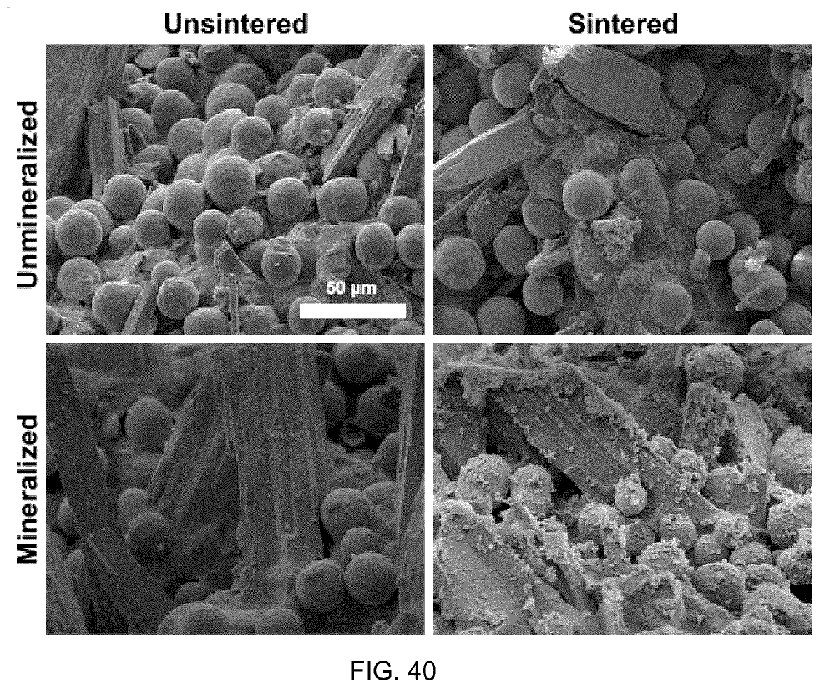

FIG. 40 displays SEM images of sintered, unsintered, mineralized, and unmineralized lattice structures. The physical analysis reveals different mineralization despite the same crystals forming.

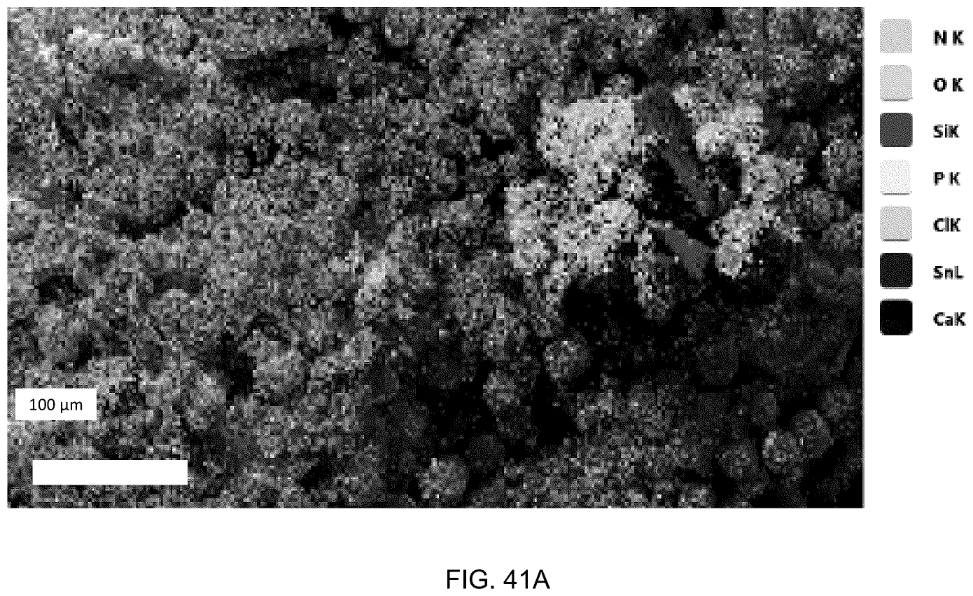

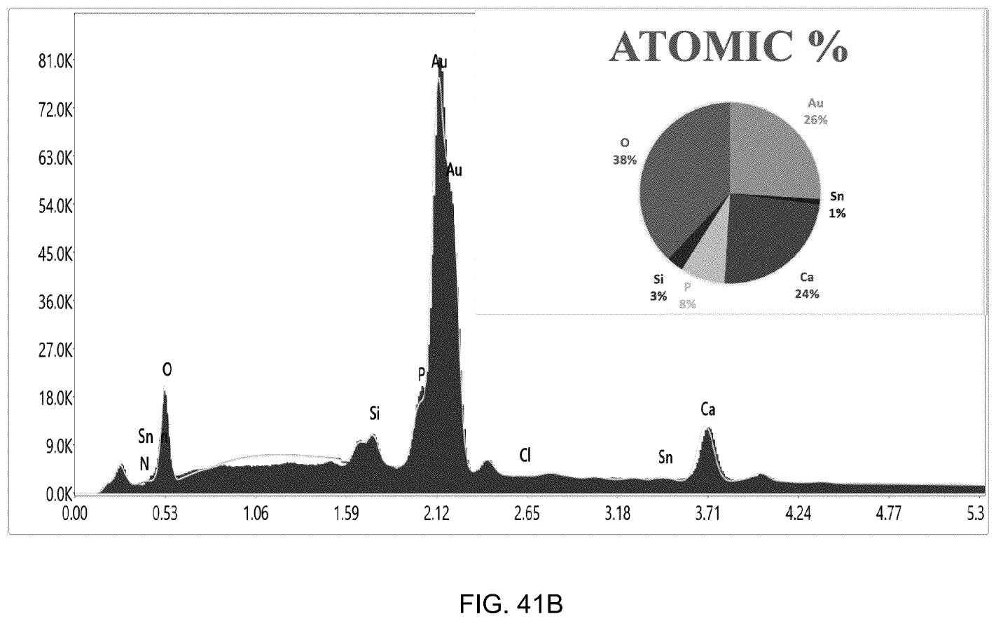

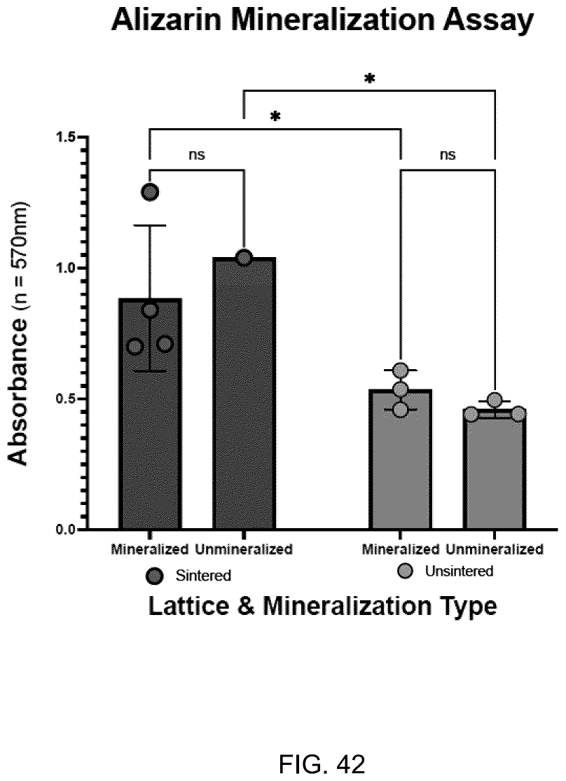

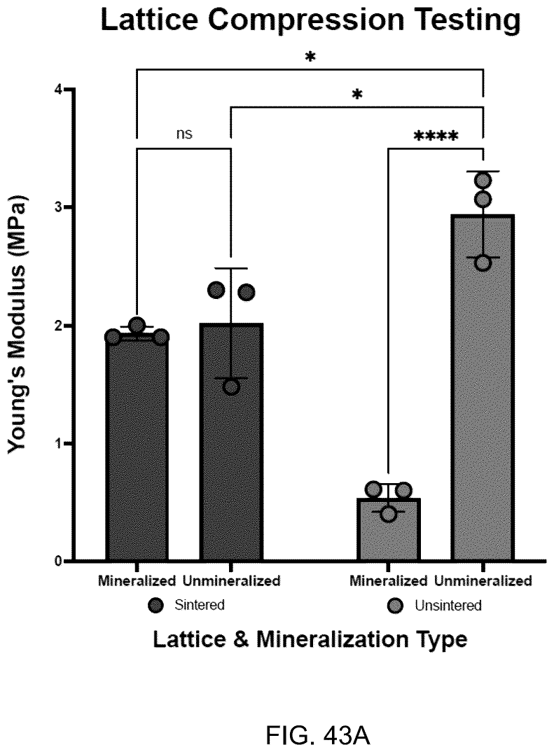

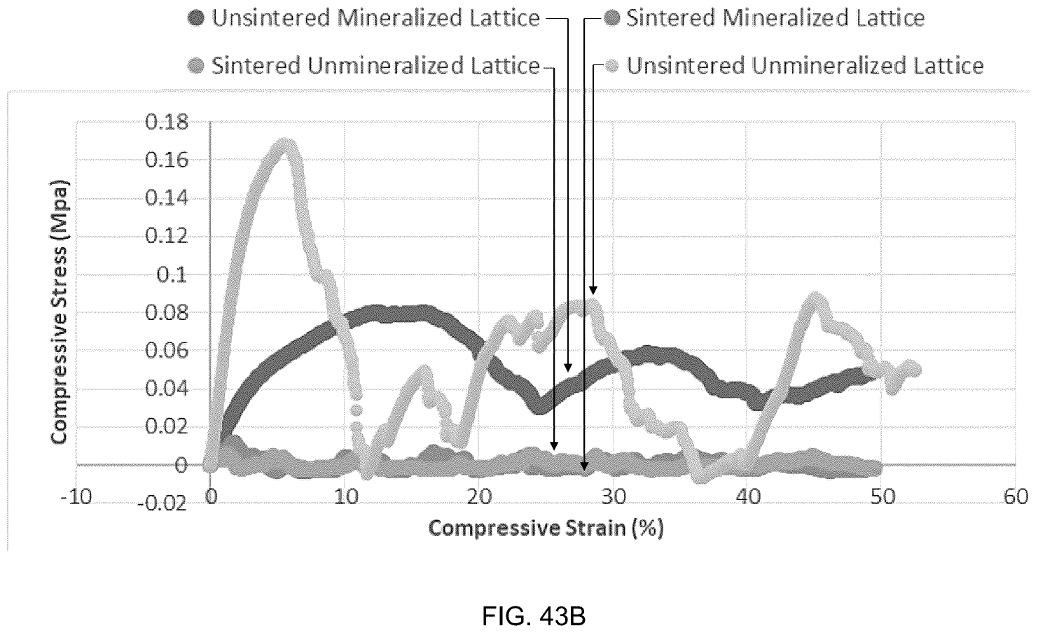

Further, FIGS. 41A, 41B, 42, 43A, and 43B display the results of an elemental, mineralization, and mechanical analysis. FIGS. 41A-41B display EDS of a sintered lattice structure. FIG. 42 displays an Alizarin mineralization assay. As shown, sintered lattice structures have higher levels of mineralization (p<0.05). FIG. 43A displays a lattice structure compression test. As shown, an unmineralized and unsintered lattice structure is the stiffest and strongest. FIG. 43B displays a compressive stress-strain curve for each condition (unsintered mineralized lattice structure, sintered mineralized lattice structure, sintered unmineralized lattice structure, and unsintered unmineralized lattice structure) (p<0.05).

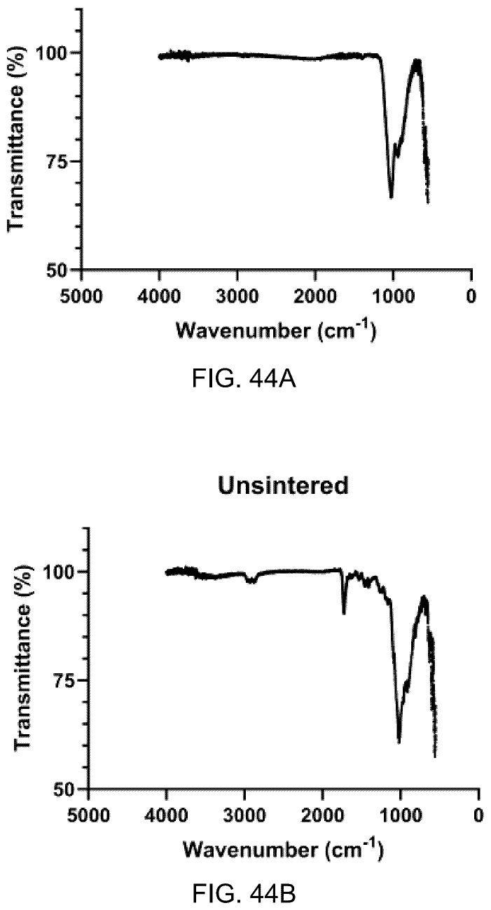

FIG. 44A displays a FTTR analysis for sintered conditions and FIG. 44B displays a FTIR analysis for unsintered conditions. As shown, the organic binder is effectively sintered off lattice structures.

Mineralization studies demonstrate that unmineralized lattice structures are the stiffest and strongest compared to all other lattice structures, where the mineralized condition resulted in scaffolds that were more elastic. Note that there was no significant difference across sintered lattice structures. XRD indicates that the same crystals are forming in both conditions. However, FTIR analysis shows that organic compounds within the binder are being effectively sintered off. SEM results demonstrate lattice structure particles breaking and reforming as compared to an unsintered lattice, but this change is not widespread. These results also indicate an elevated level of mineralization for a sintered lattice structure as compared to an unsintered lattice structure.

Although the present disclosure has been described with respect to one or more particular embodiments and/or examples, it will be understood that other embodiments and/or examples of the present disclosure may be made without departing from the scope of the present disclosure.

1. A bone graft scaffold comprising:
a lattice structure having a triply periodic minimal surface (TPMS) shape, wherein the lattice structure is 3D printed; and
a cryogel solution disposed within the lattice structure.

2. The bone graft scaffold of claim 1, wherein the lattice structure having the TPMS shape is shaped as a gyroid lattice, a diamond, an I-WP, a neovius, a primitive, a Fischer-Koch S, an F-RD, or a PMY.

3. The bone graft scaffold of claim 1, wherein the lattice structure has a solid volume from 0.5 to 150 cm³.

4. The bone graft scaffold of claim 1, wherein the lattice structure has a relative density from 10% to 50%.

5. The bone graft scaffold of claim 1, wherein the lattice structure has a pore diameter from 0.5 to 5 mm.

6. The bone graft scaffold of claim 1, wherein the lattice structure is fabricated of ceramic materials or plastic materials.

7. The bone graft scaffold of claim 6, wherein the ceramic materials are inorganic composites comprising calcium, phosphorus, silicon dioxides, and/or calcium carbonates.

8. The bone graft scaffold of claim 1, wherein the cryogel solution is made of at least one of chitosan, gelatin, silk, collagen, polyacrylamide, alginate, cellulose, laminin, fibrinogen, MXenes, polyethylene glycol (PEG), polyethylene oxide (PEO), polyvinyl alcohol (PVA), or N-Vinylpyrrolidone (NVP).

9. The bone graft scaffold of claim 8, wherein the cryogel solution is a chitosan-gelatin cryogel solution.

10. The bone graft scaffold of claim 1, wherein the cryogel solution has a porosity from 50% to 80%.

11. The bone graft scaffold of claim 1, wherein the bone graft scaffold has a porosity from 25% to 70%.

12. The bone graft scaffold of claim 1, wherein the bone graft scaffold has a relative swelling ratio from 1500% to 2500%.

13. The bone graft scaffold of claim 1, wherein the bone graft scaffold has a mechanical strength from 0.5 MPa to 100 MPa.

14. A method of making a bone graft scaffold comprising:
freezing a cryogel solution;
pouring the cryogel solution onto a lattice structure having a triply periodic minimal surface (TPMS) shape, wherein the lattice structure is 3D printed; and
freezing the cryogel solution poured onto the lattice structure to form the bone graft scaffold.

15. The method of claim 14, further comprising sintering the bone graft scaffold.

16. The method of claim 14, wherein the cryogel solution is made of at least one of chitosan, gelatin, silk, collagen, polyacrylamide, alginate, cellulose, laminin, fibrinogen, MXenes, polyethylene glycol (PEG), polyethylene oxide (PEO), polyvinyl alcohol (PVA), or N-Vinylpyrrolidone (NVP).

17. The method of claim 16, wherein the cryogel solution is a chitosan-gelatin cryogel solution.

18. The method of claim 14, wherein the cryogel solution has a porosity from 50% to 80%.

19. The method of claim 14, wherein the lattice structure having the TPMS shape is shaped as a gyroid lattice, a diamond, an I-WP, a neovius, a primitive, a Fischer-Koch S, an F-RD, or a PMY.

20. The method of claim 14, wherein the lattice structure has a solid volume from 0.5 to 150 cm³.

21. The method of claim 14, wherein the lattice structure has a relative density from 10% to 50%.

22. The method of claim 14, wherein the lattice structure has a pore diameter from 0.5 to 5 mm.

23. The method of claim 14, wherein the lattice structure is fabricated of ceramic materials or plastic materials.

24. The method of claim 23, wherein the ceramic materials are inorganic composites comprising calcium, phosphorus, silicon dioxides, and/or calcium carbonates.

25. The method of claim 14, wherein the integrated bone graft scaffold has a porosity from 25% to 70%.

26. The method of claim 14, wherein the integrated bone graft scaffold has a relative swelling ratio from 1500% to 2500%.

27. The method of claim 14, wherein the integrated bone graft scaffold has a mechanical strength from 0.5 MPa to 100 MPa.

28. The method of claim 14, wherein the freezing the cryogel comprises freezing the cryogel at a temperature from −10° C. to −80° C. for a duration from 30 minutes to 24 hours.

29. The method of claim 14, wherein the freezing the cryogel solution poured onto the lattice structure comprises a freeze-drying cycle using a lyophilizer for a duration from 6 hours to 48 hours.
